(19) United States
(12) Patent Application Publication
(10) Pub. No.: US 2025/0152736 A1
(43) Pub. Date: May 15, 2025

(54) IMMUNE SYSTEM MODULATORS AND USES THEREOF

(71) Applicant: HDT Bio Corp., Seattle, WA (US)

(72) Inventors: Lars Peter Aksel BERGLUND, Seattle, WA (US); Steven James REED, Seattle, WA (US)

(21) Appl. No.: 19/028,766

(22) Filed: Jan. 17, 2025

Related U.S. Application Data
(63) Continuation of application No. PCT/US23/70510, filed on Jul. 19, 2023.
(60) Provisional application No. 63/368,913, filed on Jul. 20, 2022.

(51) Int. Cl. A61K 48/00; A61K 38/17; A61K 38/20; A61K 38/45; A61K 39/39; A61K 47/44; A61K 47/69

(52) U.S. Cl. CPC A61K 48/0033; A61K 38/177; A61K 38/208; A61K 38/45; A61K 39/39; A61K 47/44; A61K 47/6911; C12Y 207/07048

(57) ABSTRACT

The disclosure provides compositions, methods of treatment, and methods of making and using compositions to deliver a nucleic acid to a subject. Compositions described herein include lipid carriers, optionally including an inorganic particle, capable of admixing with nucleic acids. Compositions optionally comprising a nanoparticle carrier and nucleic acid sequence(s) encoding for (i) a cytokine; and (ii) an innate immune stimulator are provided. Further provided are compositions optionally comprising a nanoparticle carrier; an innate immune stimulator; and a nucleic acid sequence encoding for a cytokine. Methods of using the compositions as a therapeutic vaccine for the treatment of a cancer are also provided.

### CROSS-REFERENCE TO RELATED APPLICATION

This application is a continuation of U.S. International Application No. PCT/US2023/070510, filed Jul. 19, 2023, which claims the benefit of priority to U.S. Provisional Patent Application No. 63/368,913, filed Jul. 20, 2022, the contents of which is incorporated herein by reference in its entirety.

### SEQUENCE LISTING

The instant application contains a Sequence Listing which has been submitted electronically in xml format and is hereby incorporated by reference in its entirety. Said xml copy, created on Dec. 5, 2023, is named 201953-724601-SL.xml and is 195 kilobytes in size.

### BACKGROUND

Cytokines are secreted proteins that modulate innate and adaptive immune systems. Challenges for use of cytokines in the treatment of various diseases and conditions include a lack of efficient delivery systems and lack of tuning cytokine gene expression to reach appropriate levels in vivo for therapeutic effects without unwanted side effects. Thus, there is a need for improved cytokine-based therapeutics for the prevention and treatment of a broad range of diseases.

### BRIEF SUMMARY

Provided herein are compositions, wherein the compositions comprise: a lipid carrier, wherein a surface of the lipid carrier comprises cationic lipids; and a nucleic acid encoding for (i) a RNA-dependent RNA polymerase and (ii) an IL-12, a lipid carrier, wherein a surface of the lipid carrier comprises cationic lipids; and a nucleic acid encoding for: (i) an RNA-dependent RNA polymerase; and (ii) an IL-12, wherein the surface of the lipid carrier and the nucleic acid form a lipid carrier-nucleic acid complex.

Provided herein are compositions, wherein the compositions comprise: a lipid carrier; and a nucleic acid encoding for: (i) a RNA-dependent RNA polymerase; (ii) a cytokine; and (iii) an innate immune response modulator.

Provided herein are compositions, wherein the compositions comprise: a lipid carrier, wherein a surface of the lipid carrier comprises cationic lipids; and a nucleic acid encoding for (i) a RNA-dependent RNA polymerase and (ii) a cytokine; and an innate immune response modulator. Further provided herein are compositions, wherein the cytokine is not an IL-12.

Provided herein are compositions, wherein the compositions comprise: a lipid carrier, wherein the lipid carrier comprises: a cationic lipid; and a hydrophobic core, wherein the hydrophobic core comprises an inorganic nanoparticle; and a nucleic acid encoding for: (i) a RNA-dependent RNA polymerase and (ii) a cytokine.

Provided herein are compositions, wherein the compositions comprise: a lipid carrier, wherein a surface of the lipid carrier comprises cationic lipids; and a nucleic acid encoding for (i) a RNA-dependent RNA polymerase and (ii) a modified IL-12, wherein the modified IL-12 comprises a linker between a p35 subunit and a p40 subunit.

Provided herein are compositions, wherein the compositions comprise: a lipid carrier; and a nucleic acid encoding for (i) a RNA-dependent RNA polymerase and (ii) a cytokine; and an innate immune response modulator, wherein the innate immune response modulator is a nucleic acid, wherein the nucleic acid comprises a region coding a sequence at least 85% identical to SEQ ID NO: 17.

Provided herein are compositions, wherein the compositions comprise: a lipid carrier; and a nucleic acid encoding for: a RNA-dependent RNA polymerase; a cytokine; and an innate immune modulator coding a sequence at least 85% identical to SEQ ID NO: 17.

Provided herein are methods for modulating an immune response in a subject, wherein the methods comprise: administering to the subject a composition provided herein.

Provided herein are methods for treating cancer in a subject, wherein the methods comprise: administering to the subject a composition provided herein.

Additional features of the present invention will be apparent to persons of ordinary skill in the art in view of the following disclosure, as well as the accompanying drawings and claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1I show schematic representations of nanoparticle (NP) carriers. FIG. 1A shows an oil-in-water emulsion and nucleic acids. FIG. 1B shows a nanostructured lipid carrier and nucleic acids. FIG. 1C shows a lipid inorganic nanoparticle and nucleic acids. FIG. 1D shows a nanoparticle comprising a cationic lipid membrane, a hydrophobic core, and nucleic acids. FIG. 1E shows a nanoparticle comprising a cationic lipid membrane, a hydrophobic core, inorganic nanoparticles within the membrane of the nanoparticle, and a plurality of nucleic acids. FIG. 1F shows a nanoparticle having a cationic lipid membrane, a liquid oil core (e.g., squalene), and two or more RNA or DNA molecules. FIG. 1G shows a nanoparticle having a cationic lipid membrane, inorganic particles, a liquid oil core, and two or more RNA or DNA molecules. FIG. 1H shows a nanoparticle having a cationic lipid membrane, a solid core (e.g., glyceryl trimyristate-dynasan), and two or more RNA or DNA molecules. FIG. 1I shows a nanoparticle having a cationic lipid membrane (e.g., phospholipids, PEG-lipid), a solid core (e.g., cholesterol, ionizable cationic lipid), and two or more RNA or DNA molecules. Schematics are not to scale.

FIGS. 2A-2B shows graphs of protein production induced by the Miglyol lipid carrier formulation, NP-3. FIG. 2A shows graphs of protein production for the first assay. FIG. 2B shows graphs of protein production for the second assay. X-axis: Conditions; Y-axis: optical density (OD).

FIGS. 3A-3B show graphs of the levels of TNF alpha (TNFα) induced by the Miglyol lipid carrier formulation, NP-3. FIG. 3A shows a graph of TNF alpha (TNFα) from the first assay. FIG. 3B shows a graph of TNF alpha (TNFα) from the second assay. X-axis: conditions; Y-axis: TNFα levels in picograms per milliliter (pg/ml).

FIGS. 4A-4B show graphs of the correlation between protein production (sec-Nano Luc) and TNF alpha stimulation induced by NP-3. FIG. 4A shows a correlation graph of the first assay from FIGS. 2A and 3A. FIG. 4B shows a correlation graph of the second assay from FIGS. 2B and 3B. X-axis: sec-NanoLuc optical density (OD); Y-axis: TNFα levels in picograms per milliliter (pg/ml).

FIGS. 5A-5F show graphs of secreted embryonic alkaline phosphatase (SEAP) levels in BALB/c mice injected intramuscularly with various embodiments of lipid carrier formulations described herein. FIG. 5A shows a graph of the SEAP levels on day 4 post-injection. FIG. 5B shows a graph of the SEAP levels on day 6 post-injection. FIG. 5C shows a graph of the SEAP levels on day 8 post-injection. FIG. 5D shows a graph of the SEAP levels on day 4 post-injection. FIG. 5E shows SEAP levels on day 6 post-injection. FIG. 5F shows SEAP levels on day 8 post-injection. X-axis: conditions, Y-axis: relative light units (RLU).

FIG. 6 is a bar chart showing the Z-average diameter and polydispersity index (PDI) for conditions 1 to 14 listed in Table 7. X-axis: Group #; Y-axis left: Z-average diameter (d-nm, bars); Y-axis right: PDI (circles).

FIGS. 7A-7F show schematic representations of nucleic acids provided herein that can be complexed with a delivery vehicle provided herein. FIG. 7A shows a VEE self-replicating messenger RNA that encode a RNA polymerase complex for amplification of the cytokine transgene in the cytoplasm of transfected cells (e.g., IL-12-SEQ ID NO: 15). FIG. 7B shows a VEE self-replicating RNA that includes a cytokine transgene and an innate immune response modulator. FIG. 7C shows a VEE self-replicating RNA that includes a cytokine transgene and an innate immune response modulator, each with their own promoter regions (P). FIG. 7D shows a first nucleic acid comprising a VEE self-replicating RNA encoding for a cytokine and a second nucleic acid encoding for an innate immune response modulator. FIG. 7E shows a first nucleic acid comprising a VEE self-replicating RNA encoding for an innate immune response modulator, and a second nucleic acid encoding for a cytokine. FIG. 7F shows a first nucleic acid comprising a VEE self-replicating RNA encoding for a cytokine and a second nucleic acid comprising a VEE self-replicating RNA encoding for an innate immune response. P=Promotor region for all figures.

FIGS. 8A-8E show graphs of the effect of NP-1 or NP-30 in complex with repRNA-IL-12 on melanoma tumor volume, animal survival, and cytokine production. FIG. 8A shows a graph of tumor volumes and survival of mice with established SC B16 melanoma tumors that were injected intratumorally with PBS or with 5 μg IL-12-repRNA-NP-1 (SEQ ID NO: 45) 10 days post-tumor-inoculation shown by arrow. X-axis: Days post-tumor inoculation; Y-axis: tumor volume (left graph), percent (%) animal survival (right graph). FIG. 8B shows a graph of the extracellular cytokines in sera 24 hour post-treatment with PBS or with 5 μg IL-12-repRNA-NP-30 (SEQ ID NO: 45). X-axis: conditions; Y-axis: level of cytokines in sera in picograms per milliliter (pg/ml). FIG. 8C shows a graph of the level of cytokines in sera 24, 48, and 72 hours post-treatment with PBS or with 5 μg IL-12-repRNA-NP-30 via cytokine capture assay. X-axis: types of extracellular cytokines measured; Y-axis: conditions; a heatmap shows relative levels of cytokines in the capture assay. FIG. 8D shows a graph of extracellular cytokines in tumors 24, 48, and 72 hours post-treatment with PBS or with 5 μg IL-12-repRNA-NP-30. X-axis: conditions; Y-axis: level of cytokines in the tumor in picograms/cm³. FIG. 8E shows graphs of tumor lymphocyte infiltration and CD4 Th1 polarization via intranuclear staining and flow cytometry post-treatment with PBS or with 5 μg IL-12-repRNA-NP-30. X-axis: conditions; Y-axis: cells per mm³.

FIGS. 9A-9C show graphs of the effect of lipid carriers in complex with repRNA-IL-12 on breast cancer tumor volume and cytokine production. FIG. 9A shows a graph of tumor volume with established breast cancer tumors that were injected intratumorally with PBS, 5 μg LNP+repRNA-IL-12, or with 5 μg IL-12-repRNA-NP-30 0 to 30 days post tumor-inoculation. X-axis: Days post-tumor inoculation; Y-axis: tumor volume (mm³). FIG. 9B-9C shows a graphs of the extracellular cytokines in sera 24 hour post-treatment. FIG. 9B shows a graph of IL-6 in sera. FIG. 9C shows a graph of interferon-alpha (IFN-α) in sera. X-axis: conditions; Y-axis: level of cytokines in sera in picograms per milliliter (pg/ml).

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Provided herein are compositions, kits, methods, and uses thereof for modulating an immune response in a subject. Briefly, further described herein are (1) nucleic acids encoding for cytokines and innate immune response modulators; (2) innate immune response modulators; (3) self-replicating nucleic acids; (4) delivery vehicles; (5) thermally stable, dried, and lyophilized vaccines; (6) pharmaceutical compositions; (7) therapeutic applications; and (8) kits.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to ±20% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve or at least partially achieve the desired effect.

### Nucleic Acids Encoding for Cytokines

Provided herein are compositions comprising a nucleic acid. In some embodiments, the nucleic acid is in complex with the nanoparticle. In some embodiments, the nucleic acid forms a complex with the cationic surface of the nanoparticle or lipid carrier provided herein. In some embodiments, the nucleic acid is in complex with the membrane of the nanoparticle. In some embodiments, the nucleic acid is in complex with the hydrophilic surface of the nanoparticle. In some embodiments, the hydrophilic surface of the nanoparticle comprises a cationic lipid for complexation with the nucleic acid. In some embodiments, the nucleic acid is within the nanoparticle. In some embodiments, the nucleic acid is within the hydrophobic core.

In some embodiments, the nucleic acid is deoxyribonucleic acid (DNA) or ribonucleic acid (RNA). The nucleic acid may be linear or include a secondary structure (e.g., a hair pin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); hoSU (5-hydroxyuridine); moSU (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Any one or any combination of these modified nucleobases may be included in the self-replicating RNA of the invention. Many of these modified nucleobases and their corresponding ribonucleosides are available from commercial suppliers. If desired, the nucleic acid can contain phosphoramidate, phosphorothioate, and/or methylphosphonate linkages. The RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap structure can provide stability and translational efficacy to the RNA molecule. The 5′ cap of the RNA molecule may be further modified by a 2′-O-Methyltransferase which results in the generation of a cap 1 structure (m7Gppp [m2′-O]N), which may further increase translation efficacy. A cap 1 structure may also increase in vivo potency.

In some embodiments, compositions provided herein comprise one or more nucleic acids. In some embodiments, compositions provided herein comprise two or more nucleic acids. In some embodiments, compositions provided herein comprise at least one DNA. In some embodiments, compositions provided herein comprise at least one RNA. In some embodiments, compositions provided herein comprise at least one DNA and at least one RNA. In some embodiments, nucleic acids provided herein are present in an amount of above 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

In some embodiments, compositions provided herein comprise one or more modulator of immune system response. In some embodiments, the composition comprises a nucleic acid encoding an immune system modulator. An immune system modulator is an agent, cytokine, or protein that changes the level of an immune cell (e.g., B-cell, T-cell, antigen presenting cell, activated B-cell, activated T-cell, activated macrophage), changes the level of immunomodulatory molecules (e.g., inflammatory cytokines, chemokines), or a combination thereof. Modulation of immune response by such an immune system modulator can be a suppression of the immune response (immunosuppression or anti-inflammatory) in a subject or an increase the immune response (immunostimulatory or pro-inflammatory) in a subject.

In some embodiments, a nucleic acid provided herein encodes for a cytokine. Cytokines are small proteins (generally about 5 to 20 kDa) that act through their corresponding target cytokine receptors to modulate immune responses, cell growth, and other cellular functions. Immune cells secrete cytokines and interferons to signal to other immune cells, e.g., to promote phagocytosis of a microorganism or infected cells, or induce inflammation at the site of an injury. In some embodiments, the cytokine is a pro-inflammatory cytokine. Non-limiting examples of pro-inflammatory cytokines include interleukin-12 (IL-12), IL-18, IL-17, IL-10, TNF-alpha (TNF-α), interferon gamma (IFNγ), and granulocyte-macrophage colony stimulating factor (GM-CSF).

In some embodiments, the cytokine is an anti-inflammatory cytokine. Non-limiting examples of anti-inflammatory cytokines include IL-4, IL-10, IL-11, IL-13, and IL-35.

In some embodiments, a nucleic acid provided herein encodes for an IL-12 family cytokine. IL-12 is generally secreted from B-cells and macrophages. IL-12 can induce proliferation of natural killer (NK) cells, increase interferon (IFN) production, and promote cell-mediated immune functions. IL-12 can induce naïve CD4+ T cells to differentiate into Th1 cells. The interleukin 12 (IL-12) family is comprised of 4 members, IL-12, IL-23, IL-27, and IL-35. IL-12, IL-23 and IL-27 are secreted by activated antigen presenting cells (APC) during antigen presentation to naïve T cells while IL-35 is a product of regulatory T and B cells. Each IL-12 family cytokine is composed of an α-subunit with a helical structure and a β-subunit structurally related to the extracellular regions of Type 1 cytokine receptors (e.g., soluble IL-6 receptor). The α-subunits are IL-12p35, IL-23p19 and IL-27p28 and the β subunits are IL-12p40 and Ebi3 and co-expression of both chains is necessary for secretion of the bioactive cytokine by an antigen presenting cell. Because the three alpha subunits (IL-12p35, IL-23p19 and IL-27p28) are structurally related, each can pair with either of the structurally homologous 3 subunits (IL-12p40 and Ebi3). In some embodiments, a nucleic acid provided herein encodes for an alpha and/or a beta chain of an IL-12 family cytokine. In some embodiments, nucleic acids provided herein encode for an IL-12p40. In some embodiments, nucleic acids provided herein encode for a Ebi3. In some embodiments, nucleic acids provided herein encode for an IL-12p35. In some embodiments, nucleic acids provided herein encode for an IL-23p19. In some embodiments, nucleic acids provided herein encode for an IL-27p28. In some embodiments, a nucleic acid provided herein encodes for (1) IL-12p35, IL-23p19, or IL-27p28; and (2) IL-12p40 or Ebi3.

In some embodiments, a nucleic acid provided herein encodes for IL-12, or a functional variant thereof. In some embodiments, a nucleic acid provided herein encodes a human IL-12A subunit, or a functional variant thereof. In some embodiments, a nucleic acid provided herein encodes a human IL-12B subunit, or a functional variant thereof. In some embodiments, a nucleic acid provided herein encodes a mouse IL-12α subunit, or a functional variant thereof. In some embodiments, a nucleic acid provided herein encodes a mouse IL-12b subunit or a functional variant thereof.

In some embodiments, a nucleic acid provided herein further comprises a sequence encoding a linker. In some embodiments, the linker comprises an amino acid sequence set forth in SEQ ID NO: 41.

In some embodiments, a nucleic acid provided herein encodes a human IL-12B, a linker, and a human IL12A subunit. In some embodiments, a nucleic acid provided herein encodes a mouse IL-12b, a linker, and a mouse IL12a subunit. In some embodiments, a nucleic acid provided herein encodes for a mouse IL-12 comprising an amino acid sequence that is at least 85% identical to any one of SEQ ID NOS: 3-4, or 6, and any combination thereof. In some embodiments, the nucleic acid encodes for a fusion protein comprising a mouse IL-12b (p40) and a mouse IL-12α (p35) subunit, wherein the fusion protein further comprises an elastin linker between subunits. In some embodiments, a nucleic acid provided herein encodes for a mouse IL-12 fusion protein, wherein the nucleic acid comprises a sequence that is at least 85% identical to SEQ ID NO: 16, SEQ ID NO: 32, or SEQ ID NO: 45. In some embodiments, the fusion protein comprises an amino acid sequence that is at least 85% identical to SEQ ID NO: 6. In some embodiments, the nucleic acid encodes for a fusion protein comprising a human IL-12B (p40) and a human IL-12A (p35) subunit, wherein the fusion protein further comprises an elastin linker between subunits. In some embodiments, the fusion protein comprises an amino acid sequence that is at least 85% identical to SEQ ID NO: 5. In some embodiments, a nucleic acid provided herein encodes for a human IL-12 comprising an amino acid sequence that is at least 85% identical to any one of SEQ ID NOS: 1-2, or 5, and any combination thereof. In some embodiments, a nucleic acid provided herein encodes for a human IL-12 fusion protein, wherein the nucleic acid comprises a sequence that is at least 85% identical to SEQ ID NO: 42 or SEQ ID NO: 43.

In some embodiments, a nucleic acid provided herein encodes for interferon gamma (IFNγ), or a functional variant thereof. In some embodiments, a nucleic acid provided herein encodes for a human IFNγ comprising an amino acid sequence that is at least 85% identical to SEQ ID NO: 7. In some embodiments, a nucleic acid provided herein encodes for a mouse IFNγ comprising an amino acid sequence that is at least 85% identical to SEQ ID NO: 8. Interferon is generally secreted from activated Th1 T-cells and natural killer (NK) cells. IFNγ can induce expression of class I MHC molecule on the surface of somatic cells, induce class II MHC expression on antigen presenting cells (APCs) and somatic cells. IFNγ can induce activation of macrophages, neutrophils, and NK cells. In addition, IFNγ promotes cell-mediated immunity and antiviral responses.

In some embodiments, a nucleic acid provided herein encodes for IL-2, or a functional variant thereof. In some embodiments, a nucleic acid provided herein encodes for a human IL-2 comprising an amino acid sequence that is at least 85% identical to SEQ ID NO: 9. IL-2 is generally secreted or expressed by activated Th1 cells and NK cells. IL-2 can induce and enhance proliferation of B cells and activated T cells. IL-2 can also modulate NK cellular functions.

In some embodiments, a nucleic acid provided herein encodes for IL-15, or a functional variant thereof. In some embodiments, a nucleic acid provided herein encodes for a human IL-15 comprising an amino acid sequence that is at least 85% identical to SEQ ID NO: 10. IL-15 is generally expressed or secreted by mononuclear phagocytes (e.g., macrophages, monocytes, Kupffer cells, histiocytes, microglia, osteoclasts, dust cells, Langerhans cells, Hofbauer cells, intraglomerular mesangial cells sinusoidal lining cells, etc.). IL-15 induces proliferation of NK cells among other functions.

In some embodiments, a nucleic acid provided herein encodes for IL-18, or a functional variant thereof. In some embodiments, a nucleic acid provided herein encodes for a human IL-18 comprising an amino acid sequence that is at least 85% identical to SEQ ID NO: 11. IL-18 is generally expressed or secreted by macrophages and can induce interferon production and expression by T cells and NK cells.

In some embodiments, a nucleic acid provided herein encodes for IL-21, or a functional variant thereof. In some embodiments, a nucleic acid provided herein encodes for a human IL-21 comprising an amino acid sequence that is at least 85% identical to SEQ ID NO: 12. IL-21 is generally secreted by T cells such as Th2 cells, T follicular cells, and NK T cells. IL-12 induces cell proliferation and activates CD8+ T cell effector activity.

In some embodiments, a nucleic acid provided herein encodes for IL-23, or a functional variant thereof. In some embodiments, a nucleic acid provided herein encodes for a human IL-23 comprising an amino acid sequence that is at least 85% identical to SEQ ID NO: 13. IL-23 is generally secreted from or expressed by activated dendritic cells, macrophages, monocytes; innate lymphoid cells, γδ T cells; and B cells. IL-23 induces the development and differentiation of effector Th17 cells, and stimulates IL-17 production and expression, among other functions.

In some embodiments, a nucleic acid provided herein encodes for IL-27, or a functional variant thereof. In some embodiments, a nucleic acid provided herein encodes for a human IL-27 comprising an amino acid sequence that is at least 85% identical to SEQ ID NO: 14, SEQ ID NO: 15, or a combination thereof. IL-27 is composed of an α chain p28 and β chain Epstein-Barr induce gene-3 (EBI3). The p28 subunit is also called IL-30. IL-27 is generally expressed or secreted by antigen presenting cells. IL-27 can induce differentiation of T cells and upregulate IL-10 secretion.

In some embodiments, a nucleic acid provided herein encodes for IL-35, or a functional variant thereof. IL-35 is a dimeric protein composed of IL-12α and IL-27β chains, which are encoded by two separate genes—IL12A and EBI3 (Epstein-Barr virus-induced gene 3), respectively. In some embodiments, a nucleic acid provided herein encodes for a human IL-27 comprising an amino acid sequence that is at least 85% identical to SEQ ID NO: 1, SEQ ID NO: 14, or a combination thereof. IL-35 is an immunosuppressive cytokine that blocks the development of Th1 and Th17 cells by limiting early T cell proliferation in a subject.

In some embodiments, a nucleic acid provided herein encodes for IL-39, or a functional variant thereof. IL-39 is a heterodimer of IL-23p19 and Epstein-Barr induce gene-3 (EBI3). IL-39 is a cytokine secreted by stimulated and activated B cells. IL-39 induces and/or expands neutrophils and can increase the secretion of B cell activation factor (BAFF), stimulating inflammation in a subject.

In some embodiments, nucleic acids provided herein encode for a cytokine listed in Table 1. In some embodiments, compositions provided herein comprise two or more nucleic acids encoding for different sequences listed in Table 1. In some embodiments, nucleic acids provided herein encode for a cytokine protein sequence comprising at least 80%, 85%, 90%, 95%, 96%, 97%, 98%, or 99% sequence identity to a sequence listed in Table 1. In some embodiments, compositions provided herein comprise two or more nucleic acids encoding different sequences listed in Table 1. In some embodiments, the nucleic acid provided herein encodes for a cytokine or a functional fragment thereof comprising at least 80%, 85%, 90%, 95%, 96%, 97%, 98%, or 99% sequence similarity to a sequence listed Table 1. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated. Table 1 lists cytokines and sequences that can be encoded by the nucleic acids provided herein.

TABLE 1
Cytokines.

| Name | SEQ ID NOS: |
|---|---|
| IL-12 | IL12A Amino Acid: SEQ ID NO: 1 |
| | IL12B Amino Acid: SEQ ID NO: 2 |
| | IL12a Mouse Amino Acid: SEQ ID NO: 3 |
| | IL12b Mouse Amino Acid: SEQ ID NO: 4 |
| | Human IL12B-A Fusion Protein Amino |
| | Acid Sequence: SEQ ID NO: 5 |
| | Mouse IL12b-a Fusion Protein Amino |
| | Acid Sequence: SEQ ID NO: 6 |
| | IL-12 fusion protein RNA sequences: |
| | SEQ ID NO: 16, SEQ ID NO: 45 |
| | (mouse IL-12), SEQ ID NO: 42 (human IL-12) |
| IFN- | Human Amino Acid: SEQ ID NO: 7 |
| gamma | Mouse Amino Acid: SEQ ID NO: 8 |
| (IFNγ) | |
| IL-2 | Human Amino Acid: SEQ ID NO: 9 |
| IL-15 | Human Amino Acid: SEQ ID NO: 10 |
| IL-18 | Human Amino Acid: SEQ ID NO: 11 |
| IL-21 | Human Amino Acid: SEQ ID NO: 12 |
| IL-23 | Human Amino Acid: SEQ ID NO: 13 |
| IL-27 | Human IL27A Amino Acid: SEQ ID NO: 14 |
| | Human IL-27B Amino Acid: SEQ ID NO: 15 |
| IL-35 | See, e.g., SEQ ID NO: 1 and SEQ ID NO: 14 |

In some embodiments, a nucleic acid provided herein comprises a nucleic acid sequence that is at least 85% identical to SEQ ID NO: 14. In some embodiments, a nucleic acid provided herein comprises a nucleic acid sequence that is at least 90%, 95%, 96%, 97%, 98%, or 99% identical to SEQ ID NO: 14. In some embodiments, a nucleic acid provided herein comprises SEQ ID NO: 14 or a functional fragment thereof. In some embodiments, a nucleic acid provided herein comprises a nucleic acid encoding for an amino acid sequence that is least 85% identical to any one of SEQ ID NOS: 1 to 15. In some embodiments, a nucleic acid provided herein comprises a nucleic acid encoding for an amino acid sequence that is least 90%, 95%, 96%, 97%, 98%, or 99% identical to any one of SEQ ID NOS: 1 to 15. In some embodiments, a nucleic acid provided herein comprises a nucleic acid encoding for any one of SEQ ID NOS: 1 to 15.

In some embodiments, a nucleic acid provided herein encodes for interferon alpha (IFN-α) and/or interferon beta (IFN-β), or a functional variant thereof. IFN-α and IFN-β are generally secreted or expressed by macrophages, neutrophils, and somatic cells. Interferons can induce antiviral effects, induce expression of class I MHC molecules on the surface of somatic cells, and activate NK cells and macrophages.

In some embodiments, a nucleic acid provided herein encodes for granulocyte-macrophage colony-stimulating factor (GM-CSF) or a functional variant thereof. GM-CSF is generally secreted or expressed by Th cells. GM-CSF induces the growth and differentiation of monocytes and dendritic cells.

In some embodiments, a nucleic acid provided herein encodes for IL-1α or a functional variant thereof. IL-1α is generally secrete or expressed by macrophages and other APCs. IL-1α co-stimulates APCs and T cells and induces inflammation, fever acute phase response, and hematopoiesis, among other functions.

In some embodiments, a nucleic acid provided herein encodes for IL-3 and/or IL-4, or a functional variant thereof. IL-3 and IL-4 are secreted by activated T cells. IL-3 induces the growth of hematopoietic progenitor cells. IL-4 can induce B-cell proliferation, eosinophil and mast cell growth, induces eosinophil and mast cell function, induces IgE and class II MHC molecule expression on B cells, and can inhibit cytokine production by monocytes and macrophages.

In some embodiments, a nucleic acid provided herein encodes for IL-5 or a functional variant thereof. IL-5 is generally secreted or expressed by Th2 cells and mast cells to induce eosinophil growth and function.

In some embodiments, a nucleic acid provided herein encodes for IL-6 or a functional variant thereof. IL-6 is generally secreted or expressed by activated Th2 cells, APCs, and somatic cells. IL-6 induces acute phase responses, B-cell proliferation, thrombopoiesis. IL-6 works synergistically with IL-1 and TNF on T cell activation.

In some embodiments, a nucleic acid provided herein encodes for IL-7 or a functional variant thereof. IL-7 is generally secreted or expressed by thymic stromal cells and marrow stromal cells to induce T cell and B cell lymphopoiesis.

In some embodiments, a nucleic acid provided herein encodes for IL-8 or a functional variant thereof. IL-8 is generally secreted or expressed by macrophages and somatic cells. IL-8 to act as a chemoattractant for neutrophils and T cells.

In some embodiments, a nucleic acid provided herein encodes for IL-9 or a functional variant thereof. IL-9 is generally secreted or expressed by T cells to induce hematopoiesis and can also have thymopoeitic effects.

In some embodiments, a nucleic acid provided herein encodes for IL-10 or a functional variant thereof. IL-10 is generally secreted or expressed by activated Th2 cells, CD8+ T cells, B cells, and macrophages. IL-10 inhibits cytokine production, promotes B cell proliferation and antibody production. IL-10 also suppresses cellular immunity and mast cell growth.

In some embodiments, a nucleic acid provided herein encodes for IL-11 or a functional variant thereof. IL-11 is generally secreted or expressed by stromal cells and mesenchymal cells. IL-11 can induce thrombopoiesis and stimulate megakaryocytopoiesis. IL-11 stimulates T-cell-dependent development of IgG-secreting B-cells in spleen.

In some embodiments, a nucleic acid provided herein encodes for IL-13 or a functional variant thereof. IL-13 is generally secreted or expressed by Th2 cells and can act synergistically with IL-4 to induce B-cell proliferation.

In some embodiments, a nucleic acid provided herein encodes for IL-17 (also called IL-17a) or a functional variant thereof. IL-17 is generally secreted or expressed by T-helper 17 (Th17) cells, a subset of CD4+ T-cell that secrete IL-17. IL-17 acts as a chemotaxis signal for monocytes and neutrophils to a site of inflammation. IL-17 mediates effects on stromal cells, resulting in production of inflammatory cytokines and recruitment of leukocytes (e.g., neutrophils), creating a link between innate and adaptive immunity.

In some embodiments, a nucleic acid provided herein encodes for IL-22 or a functional variant thereof. IL-22 is generally secreted by Th1, Th22, Th17, and γδ T cells; NK T cells; innate lymphoid cells (ILC3), neutrophils; and macrophages. IL-22 can improve cell survival and proliferation. IL-22 can also promote the synthesis of anti-microbial peptides such as S100, regenerating islet-derived protein 3-beta (Reg3β), regenerating islet-derived protein 3 gamma (Reg3γ), and defensins.

In some embodiments, a nucleic acid provided herein encodes for IL-25 (also called IL-17e) or a functional variant thereof. IL-25 is generally secreted by T cells, dendritic cells, macrophages, mast cells, basophils, eosinophils, epithelial cells and Paneth cells. IL-25 can induce NF-κB activation, the production of IL-8, and a neutrophil chemotaxis. IL-25 also activates eosinophil expansion.

In some embodiments, a nucleic acid provided herein encodes for macrophage inflammatory protein (MIP)-1α and/or MIP-1β, or a functional variant thereof. MIP-1α, also called chemokine (C-C motif) ligand 3 (CCL3), is secreted by macrophages. MIP-1β, also called chemokine (C-C motif) ligands 4 (CCL4) is secreted by lymphocytes and macrophages. MIP-1α and MIP-1β can activate granulocytes (e.g., neutrophils, eosinophils and basophils) to induce acute inflammation.

In some embodiments, a nucleic acid provided herein encodes for transforming growth factor beta (TGF-β) or a functional variant thereof. TGF-β is a multifunctional cytokine belonging to the transforming growth factor superfamily that includes three different mammalian isoforms (TGF-β 1 to 3, HGNC symbols TGFB1, TGFB2, TGFB3) and many other signaling proteins. TGF-β proteins are generally produced and secreted by leukocytes, including T cells and monocytes. TGF-β can induce chemotaxis, IL-1 synthesis, IgA synthesis, and inhibit cell proliferation.

In some embodiments, a nucleic acid provided herein encodes for a tumor necrosis factor family protein. In some embodiments, a nucleic acid provided herein encodes for tumor necrosis factor-alpha (TNF-α) or a functional variant thereof. TNF-α is generally secreted by macrophages, mast cells, NK cells, and sensory neurons. TNF-α can induce cell death, induce inflammation, and activate pain signaling. In some embodiments, a nucleic acid provided herein encodes for tumor necrosis factor-beta (TNF-0) or a functional variant thereof. TNF-β, also called lymphotoxin-alpha (LT-α), is produced and secreted by lymphocytes. TNF-β has a number of different functions depending on the form that is secreted or expressed by a cell (e.g., a soluble homotrimer or as a cell surface protein heterotrimer-LTβ). TNF-β can induce cell death and induce inflammation.

In some embodiments, a nucleic acid provided herein encodes for a linker. In some embodiments, the linker is between an alpha and a beta chain of a cytokine. In some embodiments, the linker is between an alpha and a beta chain of an IL-12 family cytokine. In some embodiments, the linker is about 14 to 18 amino acids long.

### Innate Immune Response Modulators

Provided herein are compositions comprising an innate immune response modulator. In some embodiments, the innate immune response modulator is a nucleic acid sensor engaging composition. In some embodiments, the innate immune response modulator is a pattern recognition receptor (PRR) agonist. In some embodiments, the PRR agonist is a nucleic acid. The nucleic acid may be single-stranded or double-stranded. The nucleic acid may be RNA or DNA. The nucleic acid may be linear or include a hairpin. The nucleic acid encoding the innate immune response modulator can be on the same nucleic acid strand as the nucleic acid encoding for the cytokine or on a different nucleic acid strand. In some embodiments, the innate immune response modulator is an endosomal nucleic acid sensor. In some embodiments, the innate immune response modulator is toll-like receptor (TLR) agonist. Exemplary TLRs include TLR3, TLR7, TLR8, and TLR9. In some embodiments, the TLR is TLR3. In some embodiments, the TLR3 agonist is RIBOXXOL, poly(I:C), or Hiltonol®. In some embodiments, the innate immune response modulator is a retinoic acid-inducible gene I (RIG-I)-like receptor (RLR) agonist. In some embodiments, the RLR agonist is RIG-I, melanoma differentiation-associated protein 5 (MDA5), or laboratory of genetics physiology 2 (LGP2). In some embodiments, the innate immune response modulator is a viral RNA sequence, or a functional variant thereof. In some embodiments, the innate immune response modulator comprises a triphosphate (PPP) group at the 5′ end. In some embodiments, the innate immune response modulator comprises a triphosphate (PPP) group at the 5′ end is an RNA molecule. In some embodiments, the innate immune response modulator comprises an uncapped diphosphate (PP) group at the 5′ end. In some embodiments, the innate immune response modulator comprises an uncapped diphosphate (PP) group at the 5′ end is an RNA molecule. In some embodiments, the innate immune response modulator comprises a 5′-terminal nucleotide having an unmethylated 2′-O position. In some embodiments, the innate immune response modulator binds to a carboxy-terminal domain (CTD) of an RLR. In some embodiments, the innate immune response modulator comprises nucleic acid base pairs which contact the helicase domain of an RLR. In some embodiments, the innate immune response modulator is an RLR agonist. In some embodiments, the RIG-I agonist comprises hepatitis C virus (HVC) RNA genome sequence, or a functional variant thereof. In some embodiments, the RIG-I agonist comprises Sendai virus RNA genome sequence, or a functional variant thereof. In some embodiments, the RIG-I agonist comprises any RNA genome sequence, or a functional variant thereof. In some embodiments, a composition herein includes a plurality of TLR agonists. In further embodiments, the plurality of TLR agonists have different sequences. In further embodiments, the plurality of TLR agonists comprise different RNA sequences. In further embodiments, the plurality of TLR agonists comprise different DNA sequences. In further embodiments, the plurality of TLR agonists comprise RNA and DNA sequences. In some embodiments, the TLR agonist comprises a nucleic acid coding a sequence listed in Table 2. In some embodiments, the TLR agonist comprises two or more nucleic acids coding different sequence listed in Table 2. In some embodiments, the TLR agonist is a nucleic acid comprising at least 80%, 85%, 90%, 95%, 96%, 97%, 98%, or 99% sequence identity to a sequence listed in Table 2. In some embodiments, the TLR agonist comprises two or more nucleic acids coding different sequence listed in Table 2. In some embodiments, the TLR agonist is a nucleic acid comprising at least 80%, 85%, 90%, 95%, 96%, 97%, 98%, or 99% sequence similarity to a sequence listed in Table 2.

TABLE 2
PRR Agonists

| SEQ | |
|---|---|
| ID | |
| NO: | SEQUENCES |
| 17 | RNA 5′ CCAUCCUGUUUUUUUCCCUUUUUUUUUUUCUUUUUUU |
| | UUUUUUUUUUUUUUUUUUUUUUUUUUUCUCCUUUUUUUUUCCUC |
| | UUUUUUUCCUUUUCUUUCCUUU 3′ |
| 18 | RNA 5′ GGCCAUCCUGUUUUUUUCCCUUUUUUUUUUUCUUUUU |
| | UUUUUUUUUUUUUUUUUUUUUUUUUUUUUCUCCUUUUUUUUUCC |
| | UCUUUUUUUCCUUUUCUUUCCUUUUCUA 3′ |
| 19 | RNA 5′ CCCCCCCCCCCCCCCCCCCCCCCCCCCCCCCCCCCCC |
| | CCCCCCCCCCCCCCCCCCCCCCC 3′ |
| 20 | RNA 5′ GGGGGGGGGGGGGGGGGGGGGGGGGGGGGGGGGGGGG |
| | GGGGGGGGGGGGGGGGGGGGGGGGGGGGGGGGGGGGGGGGGGGG |
| | GGGGGGGGGGGGGGGGGGG 3′ |
| 21 | RNA 5′ TGCTGCTGCT TGCAAGCAGC TTGATACCAG ACAA |
| | AGCUGG GAAUAGAAAC UUCGUAUUUU CAAAGUUUUC UUUA |
| | AUAUAU UGCAAAUAAU GCCUAACCAC CUAGGGCAGG AUUA |
| | GGGUUC CGGAGUUCAA CCAAUUAGUC CUUAAUCAGG GCAC |
| | UGUAUC CGACU 3′ |
| 22 | RNA 5′ AGUCGGAUAC AGUGCCCUGA UUAAGGACUA AUUG |
| | GUUGAA CUCCGGAACC CUAAUCCUGC CCUAGGUGGU UAGG |
| | CAUUAU UUGCAAUAUA UUAAAGAAAA CUUUGAAAAU ACGA |
| | AGUUUC UAUUCCCAGC UUUGUCUGGU 3′ |
| 23 | RNA 5′ pppGGAUCGAUCGAUCGUUCGCGAUCGAUCGAUC |
| | C-3′ |
| 24 | RNA 5′ GACGAAGACC ACAAAACCAG AUAAAAAAAA AAAA |
| | AAAAAA AAAAAAAAUA AUUUUUUUUU UUUUUUUUUU UUUU |
| | UUUAUC UGGUUUUGUG GUCUUCGUC 3′ |
| 25 | RNA 5′ ppp-GGACGUACGUUUCGACGUACGUCC 3′ |
| 26 | RNA 5′-pppAGCAAAAGCAGGGUGACAAAGACAUAAUGGAUCC |
| | AAACACUGUGUCAAGCUUUCAGGUAGAUUGCUUUCUUUGGCAUG |
| | UCCGCAAAC-3′ |
| 27 | DNA 5′ pppTAATACGACTCACTATAGGCCATCCTGTTTTTTT |
| | CCCTTTTTTTTTTCTTTTTTTTTTTTTTTTTTTTTTTTTTTTTT |
| | TTTTTCTCCTTTTTTTTTCCTCTTTTTTTCCTTTTCTTTCCTT |
| | T-3′ |

In some embodiments, a nucleic acid provided herein comprises a nucleic acid sequence that is at least 85% identical to any one of SEQ ID NOS: 17 to 27. In some embodiments, a nucleic acid provided herein comprises a nucleic acid sequence that is at least 90%, 95%, 96%, 97%, 98%, or 99% identical to any one of SEQ ID NOS: 17 to 27. In some embodiments, a nucleic acid provided herein comprises a nucleic acid sequence that is at least 85% identical to SEQ ID NO: 17. In some embodiments, a nucleic acid provided herein comprises a nucleic acid sequence that is at least 90%, 95%, 96%, 97%, 98%, or 99% identical to SEQ ID NO: 17. In some embodiments, a nucleic acid provided herein comprises a nucleic acid sequence that is at least 85% identical to SEQ ID NO: 18. In some embodiments, a nucleic acid provided herein comprises a nucleic acid sequence that is at least 90%, 95%, 96%, 97%, 98%, or 99% identical to SEQ ID NO: 18. In some embodiments, a nucleic acid provided herein comprises SEQ ID NO: 17. In some embodiments, a nucleic acid provided herein comprises SEQ ID NO: 18.

In some embodiments, an innate immune response modulator is a lipid, a polysaccharide, or a combination thereof. In some embodiments, the lipid is a TLR4 agonist. In some embodiments, the TLR4 agonist is glucopyranosyl lipid A (GLA), monophosphoryl lipid A (MPLA), lipopolysaccharide (LPS), or a derivative thereof. In some embodiments, the TLR4 agonist is glucopyranosyl lipid A (GLA). The TLR4 agonist can be admixed with any nucleic acid encoding for a cytokine provided herein; and/or a delivery vehicle provided herein. In some embodiments, the TLR4 agonist and nucleic acid provided herein are in complex with a lipid carrier or nanoparticle provided herein.

### Nucleic Acids Encoding Protein Antigens

Provided herein are nucleic acids that further comprise a sequence encoding a protein antigen or an antigen-binding protein (e.g., an antibody). In some embodiments, the sequence encoding the protein antigen or the antigen-binding protein is on the same nucleic acid as the sequence encoding for a cytokine provided herein. In some embodiments, the sequence encoding the protein antigen or the antigen-binding protein is on a different nucleic acid.

In some embodiments, the nucleic acids provided herein encode for a cancer-associated protein (also referred to as a tumor protein antigen or tumor antigen). In some embodiments, the cancer-associated protein is a surface protein, a cytosolic protein, or a transmembrane protein. In some embodiments, the cancer-associated protein is a protein that is expressed by a cancer cell. In some embodiments, the cancer-associated protein is a protein that is expressed by a microbial organism that causes a cancer (e.g., viral proteins). In some embodiments, nucleic acids provided herein encode for a protein expressed by a solid cancer cell or a blood cancer cell. In some embodiments, the solid cancer cell is a melanoma cell. In some embodiments, the protein expressed by the melanoma cell is not expressed by a non-cancer cell. Non-limiting examples of cancer-associated proteins include melanoma-associated antigen (MAGE)-A1, MAGE-A3, tyrosinase, TRP-1, NY-ESO-1, prostein, gp100/pMEL17, MART-1/MelanA, TRP-2, CEA, HER-2/neu, PSMA, BAGE, GAGE-1,2, GnT-V, 43kD protein, p15, PD-1, or CTLA-4. In some embodiments, nucleic acids provided herein encoding for a protein sequence listed in Table 3 is used as part of a treatment or prevention of melanoma. In some embodiments, a nucleic acid provided herein encodes for a cancer-associated protein listed in Table 3. In some embodiments, compositions provided herein comprise two or more nucleic acids encoding for different sequences listed in Table 3. In some embodiments, nucleic acids provided herein encode for a cancer-associated protein sequence comprising at least 80%, 85%, 90%, 95%, 96%, 97%, 98%, or 99% sequence identity to a sequence listed in Table 3. In some embodiments, compositions provided herein comprise two or more nucleic acids encoding different sequences listed in Table 3. In some embodiments, the nucleic acid provided herein encodes for a cancer-associated protein or a functional fragment thereof comprising at least 80%, 85%, 90%, 95%, 96%, 97%, 98%, or 99% sequence similarity to a sequence listed Table 3. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

TABLE 3
Cancer-Associated Proteins.

| | | Amino Acid |
|---|---|---|
| SEQ ID NO: | Reference Protein | Sequence |
| SEQ ID NO: 46 | MAGE-A1 | See |
| | | Sequences |
| | | Section |
| SEQ ID NO: 47 | MAGE-A3- melanoma- | See |
| | associated antigen | Sequences |
| | 3 | Section |
| SEQ ID NO: 48 | TRP-1 (also known | See |
| | as 5,6-dihydroxy- | Sequences |
| | indole-2-carboxylic | Section |
| | acid oxidase, DHICA | |
| | oxidase, Melanoma | |
| | antigen glycoprotein | |
| | 75, Tyrosinase- | |
| | related protein 1, | |
| | and TRYP-1) | |
| SEQ ID NO: 49 | Prostein | See |
| | | Sequences |
| | | Section |
| SEQ ID NO: 50 | Tyrosinase | KCDICTDEY |
| SEQ ID NO: 51 | Tyrosinase | YMDGTMSQV |
| SEQ ID NO: 52 | Tyrosinase | MLLAYLYQL |
| SEQ ID NO: 53 | Tyrosinase | AFLPWHRLF, |
| SEQ ID NO: 54 | Tyrosinase | SEIWRDIDF |
| SEQ ID NO: 55 | gp100/pMEL17 | YLEPGPVTA |
| SEQ ID NO: 56 | gp100/pMEL17 | KTWGQUWQV |
| SEQ ID NO: 57 | gp100/pMEL17 | ITDQVPFSV |
| SEQ ID NO: 58 | gp100/pMEL17 | VLYRYGSFSV |
| SEQ ID NO: 59 | gp100/pMEL17 | LLDGTATLRL |
| SEQ ID NO: 60 | gp100/pMEL17 | ALLAVGATK |
| SEQ ID NO: 61 | gp100/pMEL17 | MLGTHTMEV |
| SEQ ID NO: 62 | gp100/pMEL17 | LIYRRRLMK |
| SEQ ID NO: 63 | gp100/pMEL17 | ALNFPGSQK |
| SEQ ID NO: 64 | MART-1/MelanA | AAGIGILTV^{†} |
| SEQ ID NO: 65 | MART-1/MelanA | ILTVILGVL |
| SEQ ID NO: 66 | gp75/TRP-1 | MSLQRQFLR |
| SEQ ID NO: 67 | TRP-2 | SVYDFFVWL |
| SEQ ID NO: 68 | TRP-2 | LLGPGRPYR |
| SEQ ID NO: 69 | CEA | YLSGANLNL |
| SEQ ID NO: 70 | HER-2/neu | KIFGSLAFL |
| SEQ ID NO: 71 | HER-2/neu | VMAGVGSPYV |
| SEQ ID NO: 72 | HER-2/neu | IISAVVGIL |
| SEQ ID NO: 73 | PSMA | LLHETDSAV |
| SEQ ID NO: 74 | PSMA | ALFDIESKV |
| SEQ ID NO: 75 | MAGE-1 | EADPTGHSY |
| SEQ ID NO: 76 | MAGE-1 | SLFRAVITK |
| SEQ ID NO: 77 | MAGE-1 | SAYGEPRKL |
| SEQ ID NO: 78 | MAGE-2 | KMVELVHFL |
| SEQ ID NO: 79 | MAGE-2 | YLOLVFGIEV |
| SEQ ID NO: 80 | MAGE-3 | EVDPIGHLY |
| SEQ ID NO: 81 | MAGE-3 | FLWGPRALV |
| SEQ ID NO: 82 | MAGE-3 | MEVDPIGHLY |
| SEQ ID NO: 83 | BAGE | AARAVFLAL |
| SEQ ID NO: 84 | GAGE-1,2 | YRPRPRRY |
| SEQ ID NO: 85 | GnT-V | VLPDVFIRC |
| SEQ ID NO: 86 | NY-ESO-1 | QLSLLMWIT |
| SEQ ID NO: 87 | NY-ESO-1 | SLLMWITQC |
| SEQ ID NO: 88 | NY-ESO-1 | ASGPGGGAPR |
| SEQ ID NO: 89 | 43kD protein | QDLTMKYQIF |
| SEQ ID NO: 90 | p15 | (E)AYGLDFYIL |
| SEQ ID NO: 91 | Mutated beta-catenin | SYLDSGIHF^{‡} |
| SEQ ID NO: 92 | Mutated elongation | ETVSEQSNVS^{§} |
| | factor 2 | |
| SEQ ID NO: 93 | Mutated CASP-8 | FPSDSWCYF |
| | (FLICE/MACH) | |
| SEQ ID NO: 94 | MUM-1 gene product | EEKLIVVLF^{¶} |
| | mutated across | |
| | intron/exon junction | |

^{†}AAGIGILTV (SEQ ID NO: 64) is also recognized by HLA B45-1- restricted cytotoxic T lymphocyte.
^{‡}Phenylalanine (F) at position 9 is the result of mutation. The wild-type sequence is SYLDSGIHS (SEQ ID NO: 95).
^{§}Glutamine (Q) at position 6 is the result of somatic mutation. The wild-type sequence is ETVSEESNV (SEQ ID NO: 96).
^{¶}Isoleucine (I) at position 5 is the result of mutation. The wild-type sequence is EEKLSVVLF (SEQ ID NO: 97).

In some embodiments, a cancer-associated protein encoded by a nucleic acid provided herein comprises a cell membrane-contacting domain or functional fragment thereof. In some embodiments, the cell membrane-contacting domain comprises a transmembrane-binding domain, an outer cell membrane-contacting domain, or an inner cell membrane-contacting domain. In some embodiments, the cell membrane-contacting domain comprises a transmembrane-binding domain, an outer cell membrane-contacting domain, and an inner cell membrane-contacting domain. In some embodiments, the cancer-associated protein is a protein expressed by a melanoma cancer cell, a prostate cancer cell, a colon cancer cell, an ovarian cancer cell, a breast cancer cell, a pancreatic cancer cell, or a blood cell.

### Self-Replicating Nucleic Acids

Provided herein are compositions comprising a self-replicating nucleic acid. The cytokines, innate immune response modulators, cancer-associated antigens, or a combination thereof can be encoded as part of a self-replicating nucleic acid construct. In some embodiments, the self-replicating nucleic acid molecule comprises at least one or more genes selected from the group consisting of viral replicases, viral proteases, viral helicases and other nonstructural viral proteins, and also comprises 5′- and 3′-end cis-active replication sequences, and an antigenic sequence encoding for a protein provided herein (e.g., a cytokine). A subgenomic viral RNA polymerase complex that directs expression of the heterologous sequence(s) can be included in the self-replicating nucleotide sequence. For example, an RNA sequence encoding for one or more non-structural proteins of a Venezuelan equine encephalitis virus (VEE). If desired, a heterologous sequence may be fused in frame to other coding regions in the self-replicating RNA and/or may be under the control of an internal ribosome entry site (IRES).

In some embodiments, the self-replicating nucleotide sequence is a self-replicating RNA molecule. Self-replicating RNA molecules are designed so that the self-replicating RNA molecule cannot induce production of infectious viral particles. This can be achieved, for example, by omitting one or more viral genes encoding for structural proteins that are necessary for the production of viral particles in the self-replicating RNA. For example, when the self-replicating RNA molecule is based on an alphavirus, such as Sindbis virus (SIN), Semliki forest virus and Venezuelan equine encephalitis virus (VEE), one or more genes encoding for viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to an animal cell even without any proteins, lead to the production of multiple daughter RNAs by transcription from itself (or from an antisense copy of itself). The self-replicating RNA can be directly translated after delivery to a cell, and this translation provides an RNA-dependent RNA polymerase which then produces transcripts from the delivered RNA. Thus, the delivered RNA leads to the production of multiple daughter RNAs. These transcripts are antisense relative to the delivered RNA and may be translated themselves to provide in situ expression of encoded cytokines and/or innate immune response modulators, or may be transcribed to provide further transcripts with the same sense as the delivered RNA which are translated to provide in situ expression of the encoded cytokines and/or innate immune response modulators(s).

The self-replicating RNA molecules provided herein can contain one or more modified nucleotides and therefore have improved stability and be resistant to degradation and clearance in vivo, and other advantages. In some embodiments, self-replicating RNA molecules that contain modified nucleotides avoid or reduce stimulation of endosomal and cytoplasmic immune receptors when the self-replicating RNA is delivered into a cell. This permits self-replication, amplification and expression of protein to occur. This also reduces safety concerns relative to self-replicating RNA that does not contain modified nucleotides, because the self-replicating RNA that contains modified nucleotides reduce activation of the innate immune system and subsequent undesired consequences (e.g., inflammation at injection site, irritation at injection site, pain, and the like). RNA molecules produced as a result of self-replication are recognized as foreign nucleic acids by the cytoplasmic immune receptors. Thus, self-replicating RNA molecules that contain modified nucleotides can provide for efficient amplification of the RNA in a host cell and expression of cytokines and/or innate immune response modulators, as well as adjuvant effects.

In some embodiments, self-replicating RNA molecules provided herein contain at least one modified nucleotide. Modified nucleotides that are not part of the 5′ cap (e.g., in addition to the modification that are part of the 5′ cap) can be used. Accordingly, the self-replicating RNA molecule can contain a modified nucleotide at a single position, can contain a particular modified nucleotide (e.g., pseudouridine, N6-methyladenosine, 5-methylcytidine, 5-methyluridine) at two or more positions, or can contain two, three, four, five, six, seven, eight, nine, ten or more modified nucleotides (e.g., each at one or more positions). Preferably, the self-replicating RNA molecules comprise modified nucleotides that contain a modification on or in the nitrogenous base, but do not contain modified sugar or phosphate moieties. In some examples, between 0.001% and 99% or 100% of the nucleotides in a self-replicating RNA molecule are modified nucleotides. For example, 0.001%-25%, 0.01%-25%, 0.1%-25%, or 1%-25% of the nucleotides in a self-replicating RNA molecule are modified nucleotides. In other examples, between 0.001% and 99% or 100% of a particular unmodified nucleotide in a self-replicating RNA molecule is replaced with a modified nucleotide. For example, about 1% of the nucleotides in the self-replicating RNA molecule that contain uridine can be modified, such as by replacement of uridine with pseudouridine. In other examples, the desired amount (percentage) of two, three, or four particular nucleotides (nucleotides that contain uridine, cytidine, guanosine, or adenine) in a self-replicating RNA molecule are modified nucleotides. For example, 0.001%-25%, 0.01%-25%, 0.1%-25%, or 1%-25% of a particular nucleotide in a self-replicating RNA molecule are modified nucleotides. In other examples, 0.001%-20%, 0.001%-15%, 0.001%-10%, 0.01%-20%, 0.01%-15%, 0.1%-25, 0.01%-10%, 1%-20%, 1%-15%, 1%-10%, or about 5%, about 10%, about 15%, about 20% of a particular nucleotide in a self-replicating RNA molecule are modified nucleotides. It is preferred that less than 100% of the nucleotides in a self-replicating RNA molecule are modified nucleotides. It is also preferred that less than 100% of a particular nucleotide in a self-replicating RNA molecule are modified nucleotides. Thus, preferred self-replicating RNA molecules comprise at least some unmodified nucleotides.

Self-replicating RNA molecules that comprise at least one modified nucleotide can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses. Suitable synthetic methods can be used alone, or in combination with one or more other methods (e.g., recombinant DNA or RNA technology), to produce a self-replicating RNA molecule that contain one or more modified nucleotides.

Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like.

In some embodiments, nucleic acids provided herein encode for an RNA polymerase. In some embodiments, nucleic acids provided herein encode for a viral RNA polymerase. In some embodiments, nucleic acids provided herein encode for: (1) a viral RNA polymerase; and (2) a cytokine or a functional fragment thereof. In some embodiments, compositions provided herein comprise a first nucleic acid encoding for a viral RNA polymerase; and a second nucleic acid encoding for a cytokine or a functional fragment thereof. In some embodiments, nucleic acids provided herein encode for: (1) a viral RNA polymerase; and (2) an innate immune response modulator or a functional fragment thereof. In some embodiments, compositions provided herein comprise a first nucleic acid encoding for a viral RNA polymerase; and a second nucleic acid encoding for an innate immune response modulator or a functional fragment thereof. In some embodiments, nucleic acids provided herein encode for: (1) a viral RNA polymerase; (2) a cytokine or a functional fragment thereof; and (3) an innate immune response modulator or a functional fragment thereof. In some embodiments, compositions provided herein comprise a first nucleic acid encoding for a viral RNA polymerase; and an innate immune response modulator or a functional fragment thereof; and a second nucleic acid encoding for a cytokine. In some embodiments, compositions provided herein comprise a first nucleic acid encoding for a viral RNA polymerase; and a cytokine or a functional fragment thereof; and a second nucleic acid encoding for an innate immune response modulator. In some embodiments, compositions provided herein comprise a first nucleic acid encoding for a viral RNA polymerase; and a cytokine or a functional fragment thereof; and a second nucleic acid encoding for viral RNA polymerase; and an innate immune response modulator. In some embodiments, compositions provided herein comprise a first nucleic acid encoding for a viral RNA polymerase; and a second nucleic acid encoding for an innate immune response modulator or a functional fragment thereof; and a third nucleic acid encoding for a cytokine.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

In some embodiments, a nucleic acid described herein comprises a sequence encoding for a cytokine provided herein and for an RNA-dependent RNA polymerase. In some embodiments, the nucleic acid region encoding for the cytokine is downstream of a subgenomic promoter from an alphavirus. In some embodiments, the nucleic acid comprises a region encoding for an alphavirus non-structural polyprotein. In some embodiments, the RNA-dependent RNA polymerase. In some embodiments, the RNA-dependent RNA polymerase includes a sub-genomic sequence from an alphavirus. In some embodiments, the RNA-dependent RNA polymerase is a VEEV RNA polymerase. In some embodiments, the two nucleic acid coding elements are present in separate nucleic acids. In some embodiments, the two nucleic acid coding elements are present on the same nucleic acid. An RNA polymerase provided herein can include but is not limited to an alphavirus RNA polymerase, an Eastern equine encephalitis virus (EEEV) RNA polymerase, a Western equine encephalitis virus (WEEV), Venezuelan equine encephalitis virus (VEEV), Also, Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 28. In some embodiments, the nucleic acid encoding for the RNA polymerase comprises at least 90% identity to the nucleic acid sequence of SEQ ID NO: 28. In some embodiments, the nucleic acid encoding for the RNA polymerase comprises at least 95% identity to the nucleic acid sequence of SEQ ID NO: 28. In some embodiments, the nucleic acid encoding for the RNA polymerase comprises at least 99% identity to the nucleic acid sequence of SEQ ID NO: 28. In some embodiments, the nucleic acid encoding for the RNA polymerase is SEQ ID NO: 28.

In some embodiments, the amino acid sequence for VEEV RNA polymerase comprises at least 85% identity to: RELPVLDSAAFNVECFKKYACNNEYWETFKENPIRLTEENVVNYITKLKGP (SEQ ID NO: 29), TQMRELPVLDSAAFNVECFKKYACNNEYWETFKENPIRLTE (SEQ ID NO: 30), or SEQ ID NO: 31. In some embodiments, the amino acid sequence for VEEV RNA polymerase comprises at least 90% identity to SEQ ID NO: 29, SEQ ID NO: 30, or SEQ ID NO: 31. In some embodiments, the amino acid sequence for VEEV RNA polymerase comprises at least 95% identity to SEQ ID NO: 29, SEQ ID NO: 30, or SEQ ID NO: 31. In some embodiments, the amino acid sequence for VEEV RNA polymerase comprises at least 99% identity to SEQ ID NO: 29, SEQ ID NO: 30, or SEQ ID NO: 31. In some embodiments, the amino acid sequence for VEEV RNA polymerase is SEQ ID NO: 29, SEQ ID NO: 30, or SEQ ID NO: 31. In some embodiments, the amino acid sequence for a VEEV RNA polymerase complex comprises SEQ ID NO: 31, or a functional variant thereof.

Provided herein are compositions and methods comprising replicon RNA (repRNA) encoding for one or more structural proteins from a non-enveloped virus. In some embodiments, the repRNA encodes a protease. In some embodiments, the repRNA encodes the 3CD protease. In some embodiments, the structural protein and the protease are co-expressed. In further embodiments, the repRNA comprises one or more open reading frames. In some embodiments, the open reading frames are separated by an internal ribosomal entry site (IRES). In some embodiments, the open reading frames are separated by a ribosomal skipping peptide sequence. In some embodiments the ribosomal skipping peptide sequence is from Thosea asigna virus (T2A).

### Delivery Vehicles

Provided herein are various compositions comprising a lipid carrier or a plurality of nanoparticles. Nanoparticles are also referred to herein as carriers, lipid carriers (when comprising a lipid) or abbreviated as NPs. Nanoparticles provided herein may be an organic, inorganic, or a combination of inorganic and organic materials that are less than about 1 micrometer (μm) in diameter. In some embodiments, nanoparticles provided herein are used as a delivery system for a bioactive agent provided herein (e.g., a nucleic acid comprising or encoding for an antigen, a pattern recognition receptor (PRR) agonist, a cytokine, or an interleukin). In some embodiments, nanoparticles provided herein are used as a delivery system for a plurality of bioactive agents. In some embodiments, compositions provided herein are vaccine compositions.

Further provided herein are various compositions comprising lipid carrier complexes or nanoparticle-complexes, wherein a plurality of lipid carriers or a plurality of nanoparticles interact physically, chemically, and/or covalently with a nucleic acid provided herein and/or other nanoparticles. The specific type of interaction between lipid carriers or between nanoparticles will depend upon the characteristic shapes, sizes, chemical compositions, physical properties, and physiologic properties. Nanoparticles provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

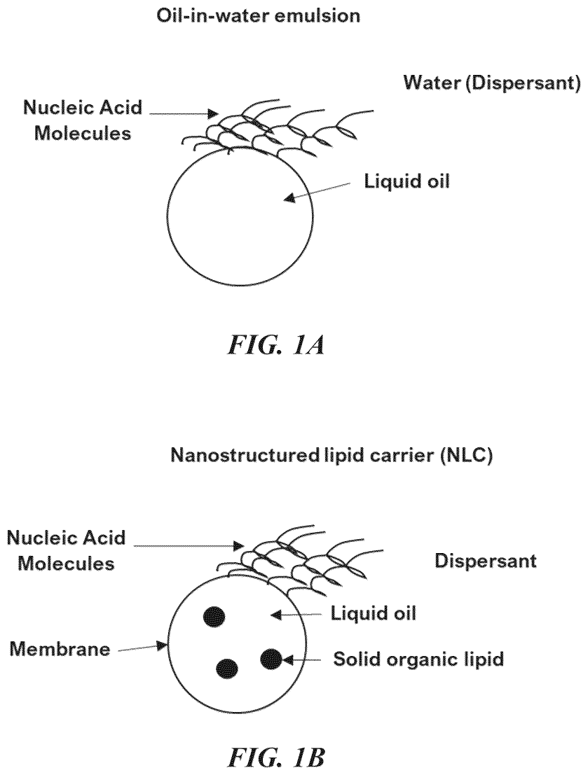

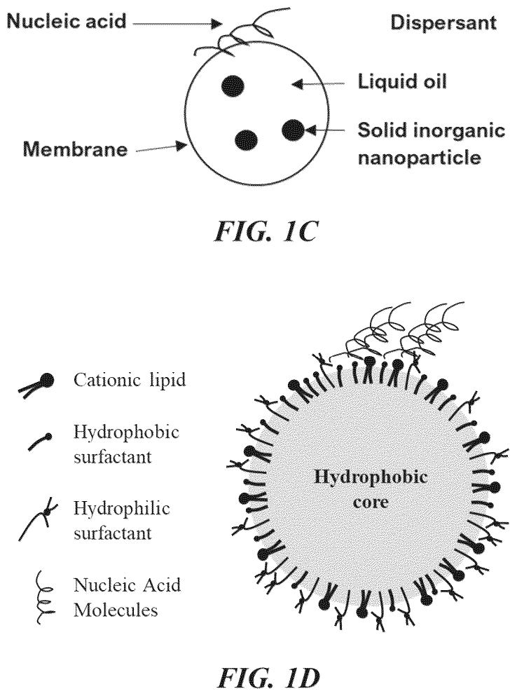

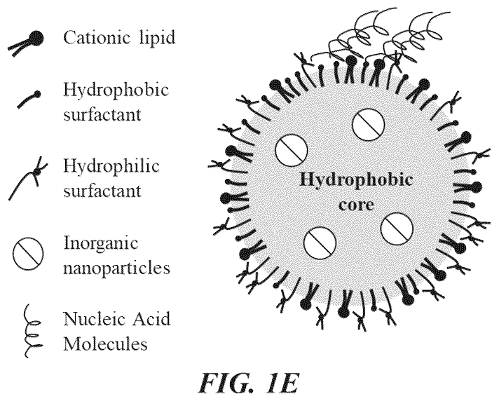

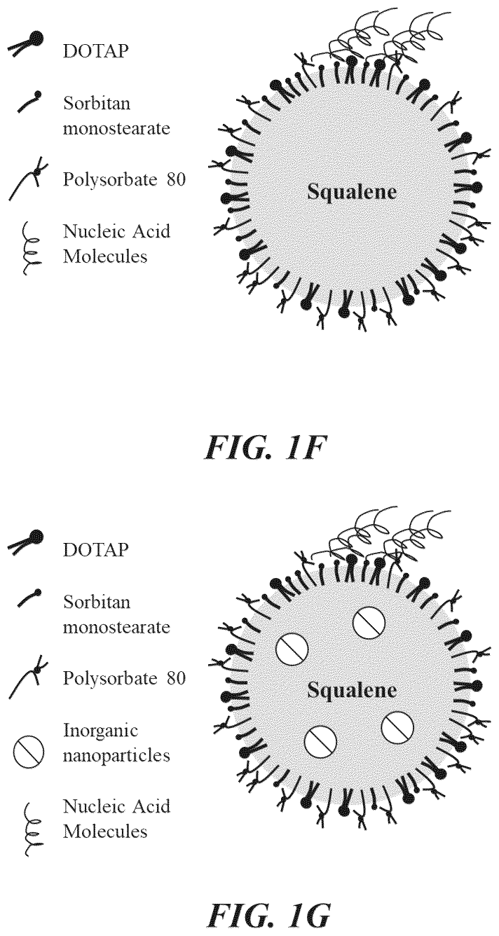

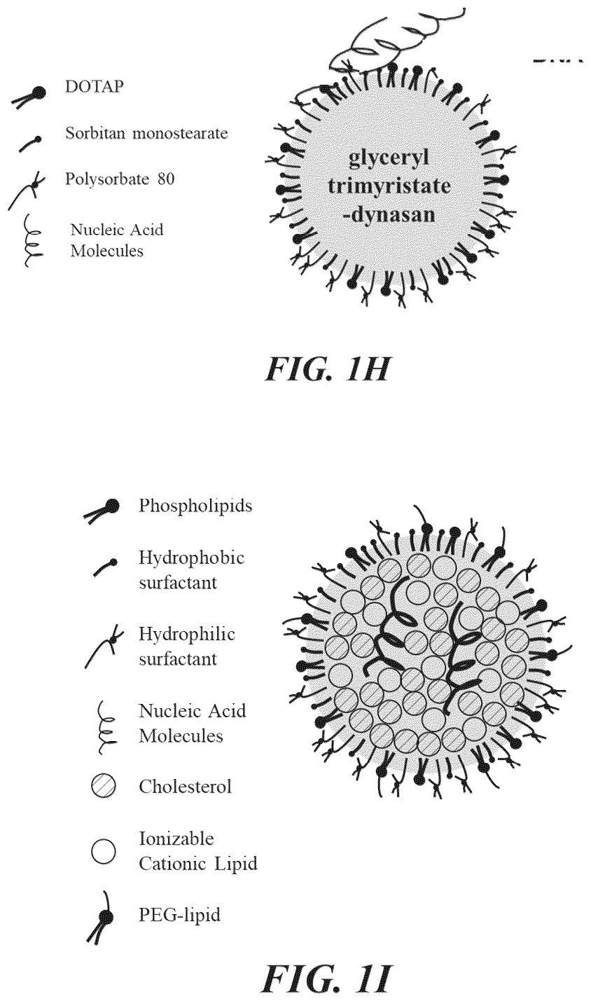

Various nanoparticles and formulations of nanoparticles (i.e., nanoemulsions) are employed. Exemplary nanoparticles are illustrated in FIGS. 1A-1I. Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. The entire nanodroplet is then dispersed as a colloid in the aqueous (water) phase. FIG. 1D (not to scale), illustrates a nanostructured lipid carrier (NLCs) comprising cationic lipids, hydrophobic surfactants, hydrophilic surfactants forming a hydrophobic core. A surface of the NLC forms a complex with a plurality of nucleic acids (nucleic acid-nanoparticle complexes and). The entire nanodroplet is dispersed in the aqueous (water) phase. FIG. 1E (not to scale), illustrates NLCs of FIG. 1D comprising solid inorganic nanoparticles within the hydrophobic core. FIG. 1F (not to scale), illustrates a nanoparticle comprising a cationic lipid membrane (e.g., DOTAP), a liquid oil core (e.g., squalene) without an inorganic particle, and one or more nucleic acids, wherein the one or more nucleic acids are in complex with the membrane. In some embodiments, nanoparticle of FIG. 1F further comprises iron oxide nanoparticles within the core as shown in FIG. 1G (not to scale). In some embodiments, a nanoparticle provided herein comprises a solid core comprising glyceryl trimyristate-dynasan (FIG. 1H). In some embodiments, a nanoparticle provided herein comprises a solid core comprising an ionizable cationic lipid and cholesterol (FIG. 1I). In some embodiments, the nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, the nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP); N-[1-(2,3-dioleyloxy)propyl]N,N,Ntrimethylammonium, chloride (DOTMA); N,N-dioleoyl-N,N-dimethylammonium chloride (DODAC), 1,2-dioleoyl-sn-glycero-3-ethylphosphocholine (DOEPC); 1,2-dioleoyl-3-dimethylammonium-propane (DODAP); 1,2-dilinoleyloxy-3-dimethylaminopropane (DLinDMA); 1,1′-((2-(4-(2-((2-(bis(2-hydroxy-dodecyl)amino)ethyl)(2-hydroxydodecyl)amino)ethyl)piperazin-1-yl)ethyl)azanediyl)bis(dodecan-2-ol) (C12-200); 306Oi10; tetrakis(8-methylnonyl) 3,3′,3″,3′″-(((methylazanediyl)bis(propane-3,1 diyl))bis (azanetriyl))tetrapropionate; 9A1P9, decyl (2-(dioctylammonio)ethyl) phosphate; A2-Iso5-2DC18; ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159; 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12; 3,6-bis(4-(bis(2-hydroxydodecyl)amino)butyl)piperazine-2,5-dione; DC-Cholesterol; 3β-[N—(N′,N′-dimethylaminoethane)-carbamoyl]cholesterol; DLin-MC3-DMA; (6Z,9Z,28Z,31Z)-heptatriaconta-6,9,28,31-tetraen-19-yl 4-(dimethylamino) butanoate; DOPE; 1,2-dioleoyl-sn-glycero-3-phosphoethanolamine; DOSPA; 2,3-dioleyloxy-N-[2-(sperminecarboxamido)ethyl]-N,N-dimethyl-1-propanaminium trifluoroacetate; DSPC; 1,2-distearoyl-sn-glycero-3-phosphocholine; ePC, ethylphosphatidylcholine; FTT5; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102); heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG; (R)-2,3-bis(myristoyloxy)propyl-1-(methoxy poly(ethylene glycol)2000) carbamate; TT3; or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticles provided herein comprise a hydrophobic core. In some embodiments, the nanoparticles provided herein comprises an oil. In some embodiments, the hydrophobic core comprises a lipid in liquid phase at 20° C., 25° C., 30° C., 35° C., 37° C., 40° C., 45° C., or 50° C. In some embodiments, the oil is in liquid phase. In some embodiments, the oil is in liquid phase at 25 degrees Celsius. Non-limiting examples of oils that can be used include α-tocopherol, coconut oil, dihydroisosqualene (DHIS), farnesene, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, vitamin E, or combinations thereof. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins. In some embodiments, the nanoparticle comprises a triglyceride ester of saturated coconut or palm kernel oil derived caprylic and capric fatty acids and plant derived glycerol, e.g., Miglyol 812 N.

In some embodiments, the hydrophobic core comprises a lipid in solid phase at 20° C., 25° C., 30° C., 35° C., 37° C., 40° C., 45° C., or 50° C. In some embodiments, the hydrophobic core comprises glyceryl trimyristate-dynasan or a derivative thereof.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. FIG. 1E illustrates an embodiment where the solid inorganic particle is within the hydrophobic core. In some embodiments, the nanoparticle provided herein comprises a metal. In some embodiments, the nanoparticle provided herein comprises a metal within the hydrophobic core. The metal can be without limitation, a metal salt such as a transition metal salt, a metal oxide such as a transition metal oxide, a metal hydroxide such as a transition metal hydroxide, a metal phosphate such as a transition metal phosphate, or a metalloid (e.g., silicon and silicon-based compounds or alloys). In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal). In some embodiments, the inorganic particle is a transition metal oxide. In some embodiments, the transition metal is magnetite (Fe₃O₄), maghemite (y-Fe₂O₃), wüstite (FeO), or hematite (alpha (α)-Fe₂O₃). In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, lipid nanoparticles described herein comprise a reporter element allowing for imaging and tracking the particle in the body of a subject. For instance, the lipid nanoparticles may comprise an inorganic solid nanoparticle detectable via magnetic resonance imaging (MRI), such as a paramagnetic, superparamagnetic, ferrimagnetic or ferromagnetic compound. In some embodiments, the inorganic solid nanoparticle materials that are MRI-detectable are iron oxides, iron gluconates, and iron sulfates.

In some embodiments, nanoparticle provided herein comprises a cationic lipid, an oil, and an inorganic particle. In some embodiments, the nanoparticle provided herein comprises DOTAP; squalene; and/or glyceryl trimyristate-dynasan; and iron oxide. In some embodiments, the nanoparticle provided herein further comprises a surfactant.

In some embodiments, the nanoparticles provided herein comprise a cationic lipid, an oil, an inorganic particle, and a surfactant. Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85).

Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5. In some embodiments, nanoparticles provided herein comprise a ratio of the esters that yields a hydrophilic-lipophilic balance between 8 and 11. HLB is used to categorize surfactants as hydrophilic or lipophilic. The HLB scale provides for the classification of surfactant function calculated e.g., by Griffin's method:

$$H L B = \frac{20 {M}_{h}}{M} ,$$

where Mₕ is the molecular mass of the hydrophilic portion of the lipid carrier and M is the molecular mass of the lipid carrier. The HLB scale is provided below:
HLB=0: fully lipophilic/hydrophobic carrier;
HLB between 0 and 6 is an oil soluble carrier;
HLB between 6 and 9 is a water dispersible carrier;
HLB between 9 and 20 is a hydrophilic, water soluble carrier;
HLB=20: fully hydrophilic/lipophobic carrier.

In some embodiments, the nanoparticle provided herein comprises a hydrophilic surfactant, also called an emulsifier. In some embodiments, the nanoparticle provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as TWEEN®, Kolliphor, Scattics, Alkest, or Canarcel; polyoxyethylene sorbitan ester (polysorbate); polysorbate 80 (polyoxyethylene sorbitan monooleate, or TWEEN® 80); polysorbate 60 (polyoxyethylene sorbitan monostearate, or TWEEN® 60); polysorbate 40 (polyoxyethylene sorbitan monopalmitate, or TWEEN® 40); and polysorbate 20 (polyoxyethylene sorbitan monolaurate, or TWEEN® 20). In one embodiment, the hydrophilic surfactant is polysorbate 80.

Nanoparticles provided herein comprises a hydrophobic core surrounded by a lipid membrane (e.g., a cationic lipid such as DOTAP). In some embodiments, the hydrophobic core comprises: one or more inorganic particles; a phosphate-terminated lipid; and a surfactant.

Inorganic solid nanoparticles described herein may be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises a surfactant such as a phosphorous-terminated surfactant, a carboxylate-terminated surfactant, a sulfate-terminated surfactant, or an amine-terminated surfactant. Typical carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate. In some embodiments, the hydrophobic core comprises one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 20 to about 6t w/v squalene, from about 0.01% to about 1m w/v iron oxide nanoparticles, from about 0.2% to about 1% w/v DOTAP, from about 0.25% to about 1% w/v sorbitan monostearate, and from about 0.5%) to about 5% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 1% w/v DOTAP, from about 0.25% to about 5% w/v sorbitan monostearate, and from about 0.5% to about 10% w/v polysorbate 80. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80. In some embodiments, a composition described herein comprises at least one nanoparticle formulation as described in Table 4. In some embodiments, a composition described herein comprises any one of NP-1 to NP-31. In some embodiments, a composition described herein comprises any one of NP-1 to NP-37.

TABLE 4
Nanoparticle Formulations.

| | | | | Additional |
|---|---|---|---|---|
| | Cationic Lipid(s) | Oil(s) | Surfactant(s) | Ingredients |
| | % (w/v) | % (w/v) | % (w/v) | % (w/v), |
| Name | or mg/ml | or mg/ml | or mg/ml | mg/ml, or mM |
| NP-1 | 30 mg/ml 1,2- | 37.5 mg/ml | 37 mg/ml | 0.2 mg Fe/ml 12 nm |
| | dioleoyl-3- | squalene | sorbitan | oleic acid-coated iron |
| | trimethylammonium- | | monostearate, | oxide nanoparticles |
| | propane (DOTAP) | | (2R)-2- | 10 mM sodium citrate |
| | chloride | | [(2R,3R,4S)-3,4- | dihydrate. |
| | | | Dihydroxyoxolan- | |
| | | | 2-yl]-2-hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-2 | 30 mg/ml 1,2- dioleoyl-3- | 37.5 mg/ml | 37 mg/ml | 1 mg Fe/ml 15 nm |
| | trimethylammonium- | squalene | sorbitan | oleic acid-coated iron |
| | propane (DOTAP) | | monostearate | oxide nanoparticles |
| | chloride | | (2R)-2- | 10 mM sodium citrate |
| | | | [(2R,3R,4S)-3,4- | dihydrate |
| | | | Dihydroxyoxolan- | |
| | | | 2-yl]-2- | |
| | | | hydroxyethyl | |
| | | | octadecenoate | |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-3 | 30 mg/ml 1,2- dioleoyl-3- | 37.5 mg/ml | 37 mg/ml | 0.2 mg Fe/ml 15 nm |
| | trimethylammonium- | Miglyol 812N | sorbitan | oleic acid-coated iron |
| | propane (DOTAP) | (triglyceride ester | monostearate, | oxide nanoparticles |
| | chloride | of saturated | (2R)-2- | 10 mM sodium citrate |
| | | coconut/palm | [(2R,3R,4S)-3,4- | dihydrate |
| | | kernel oil derived | Dihydroxyoxolan- | |
| | | caprylic and | 2-yl]-2- | |
| | | capric fatty acids | hydroxyethyl | |
| | | and plant derived | octadecenoate | |
| | | glycerol) | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-4 | 30 mg/ml 1,2- dioleoyl-3- | 37.5 mg/ml | 37 mg/ml | 1 mg Fe/ml 15 nm |
| | trimethylammonium- | Miglyol 812N | sorbitan | oleic acid-coated iron |
| | propane (DOTAP) | (triglyceride ester | monostearate, | oxide nanoparticles |
| | chloride | of saturated | (2R)-2- | 10 mM sodium citrate |
| | | coconut/palm | [(2R,3R,4S)-3,4- | dihydrate. |
| | | kernel oil derived | Dihydroxyoxolan- | |
| | | caprylic and | 2-yl]-2- | |
| | | capric fatty acids | hydroxyethyl | |
| | | and plant derived | octadecenoate, | |
| | | glycerol) | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-5 | 30 mg/ml DOTAP | 37.5 mg/ml | 37 mg/ml | 1 mg/ml |
| | chloride | squalene | sorbitan | trioctylphosphine |
| | | | monostearate | oxide (TOPO)-coated |
| | | | (SPAN ® 60) | aluminum hydroxide |
| | | | 37 mg/ml | (Alhydrogel ® 2%) |
| | | | polysorbate 80 | particles |
| | | | (TWEEN ® 80) | 10 mM sodium citrate |
| | | | | dihydrate |
| NP-6 | 30 mg/ml DOTAP | 37.5 mg/ml | 37 mg/ml | 0.2 mg Fe/ml oleic |
| | chloride | Solanesol | sorbitan | acid-coated iron oxide |
| | | (Cayman | monostearate | nanoparticles |
| | | chemicals) | (SPAN ® 60) | 10 mM sodium citrate |
| | | | 37 mg/ml | |
| | | | polysorbate 80 | |
| | | | (TWEEN ® 80) | |
| NP-7 | 30 mg/ml DOTAP | 37.5 mg/ml | 37 mg/ml | 10 mM sodium citrate |
| | chloride | squalene | sorbitan | |
| | | 2.4 mg/ml | monostearate | |
| | | Dynasan 114 | (SPAN ® 60) | |
| | | | 37 mg/ml | |
| | | | polysorbate 80 | |
| | | | (TWEEN ® 80) | |
| NP-8 | 4 mg/ml DOTAP chloride | 43 mg/ml | 5 mg/ml sorbitan | 10 mM sodium citrate |
| | | squalene | trioleate | |
| | | | (SPAN ® 85) | |
| | | | 5 mg/ml | |
| | | | polysorbate 80 | |
| | | | (TWEEN ® 80) | |
| NP-9 | 7.5 mg/ml 1,2- dioleoyl- | 9.4 mg/ml | 9.3 mg/ml | 10 mM sodium citrate |
| | 3- trimethylammonium- | squalene | sorbitan | 0.05 mg/ml 15 |
| | propane (DOTAP) | ((6E,10E,14E, | monostearate | nanometer |
| | chloride | 18E)- | (2R)-2- | superparamagnetic |
| | | 2,6,10,15,19,23- | [(2R,3R,4S)-3,4- | iron oxide (Fe₃O₄) |
| | | Hexamethyl- | Dihydroxyoxolar- | dihydrate |
| | | tetracosa- | 2-yl]-2- | |
| | | 2,6,10,14,18,22- | hydroxyethyl | |
| | | hexaene, C₃₀H₅₀) | octadecenoate, | |
| | | 0.63 mg/ml | C₂₄H₄₆O₆) | |
| | | glyceryl | (SPAN ® 60) | |
| | | trimyristate- | 9.3 mg/ml | |
| | | dynasan | polyoxyethylene | |
| | | (DYNASAN | (20) sorbitan | |
| | | 114 ®) | monooleate, | |
| | | | C₆₄H₁₂₄O₂₆, | |
| | | | Polysorbate 80 | |
| | | | (TWEEN ® 80) | |
| NP-10 | 0.4% DOTAP | 0.25% glyceryl | 0.5% sorbitan | |
| | | trimyristate- | monostearate | |
| | | dynasan | (SPAN ® 60) | |
| | | (DYNASAN | 0.5% polysorbate | |
| | | 114 ®) | 80 (TWEEN ® | |
| | | 4.75% Squalene | 80) | |
| NP-11 | 3.0% DOTAP | 0.25% glyceryl | 3.7% sorbitan | |
| | | trimyristate- | monostearate | |
| | | dynasan | (SPAN ® 60) | |
| | | (DYNASAN | 3.7% polysorbate | |
| | | 114 ®) | 80 (TWEEN ® | |
| | | 3.75% Squalene | 80) | |
| NP-12 | 0.4% DOTAP | 4.3% Squalene | 0.5% sorbitan | |
| | | | trioleate | |
| | | | (SPAN ® 85) | |
| | | | 0.5% polysorbate | |
| | | | 80 (TWEEN ® | |
| | | | 80) | |
| NP-13 | 0.4% DOTAP | 0.25% glyceryl | 2.0% polysorbate | |
| | | trimyristate- | 80 (TWEEN ® | |
| | | dynasan | 80) | |
| | | (DYNASAN | | |
| | | 114 ®) | | |
| | | 4.08% squalene | | |
| NP-14 | 0.4% DOTAP | 0.25% glyceryl | 0.5% sorbitan | |
| | | trimyristate- | trioleate | |
| | | dynasan | (SPAN ® 85) | |
| | | (DYNASAN | 2.0% polysorbate | |
| | | 114 ®) | 80 (TWEEN ® | |
| | | 4.08% squalene | 80) | |
| NP-15 | 0.4% DOTAP | 0.25% glyceryl | 0.25% sorbitan | |
| | | trimyristate- | trioleate | |
| | | dynasan | (SPAN ® 85) | |
| | | (DYNASAN | 2.0% polysorbate | |
| | | 114 ®) | 80 (TWEEN ® | |
| | | 4.08% squalene | 80) | |
| NP-16 | 0.4% DOTAP | 5% squalene | 0.5% sorbitan | |
| | | | trioleate | |
| | | | (SPAN ® 85) | |
| | | | 2.0% polysorbate | |
| | | | 80 (TWEEN ® | |
| | | | 80) | |
| NP-17 | 0.4% DOTAP | 5% squalene | 0.5% sorbitan | |
| | | | monostearate | |
| | | | (SPAN ® 60) | |
| | | | 2% polysorbate | |
| | | | 80 (TWEEN ® | |
| | | | 80) | |
| NP-18 | 0.4% DOTAP | 0.25% glyceryl | 2% sorbitan | |
| | | trimyristate- | trioleate | |
| | | dynasan | (SPAN ® 85) | |
| | | (DYNASAN | 2% polysorbate | |
| | | 114 ®) | 80 (TWEEN ® | |
| | | 4.08% squalene | 80) | |
| NP-19 | 0.4% DOTAP | 0.25% glyceryl | 0.5% sorbitan | 1% aluminum |
| | | trimyristate- | monostearate | hydroxide |
| | | dynasan | (SPAN ® 60) | |
| | | (DYNASAN | 0.5% polysorbate | |
| | | 114 ®) | 80 (TWEEN ® | |
| | | 4.75% Squalene | 80) | |
| NP-20 | 3.0% DOTAP | 0.25% glyceryl | 3.7% sorbitan | 1% aluminum |
| | | trimyristate- | monostearate | hydroxide |
| | | dynasan | (SPAN ® 60) | |
| | | (DYNASAN | 3.7% polysorbate | |
| | | 114 ®) | 80 (TWEEN ® | |
| | | 3.75% Squalene | 80) | |
| NP-21 | 0.4% DOTAP | 4.3% Squalene | 0.5% sorbitan | 1% aluminum |
| | | | trioleate | hydroxide |
| | | | (SPAN ® 85) | |
| | | | 0.5% polysorbate | |
| | | | 80 (TWEEN ® 80 | |
| NP-22 | 0.4% DOTAP | 0.25% glyceryl | 2.0% polysorbate | 1% aluminum |
| | | trimyristate- | 80 (TWEEN ® | hydroxide |
| | | dynasan | 80) | |
| | | (DYNASAN | | |
| | | 114 ®) | | |
| | | 4.08% squalene | | |
| NP-23 | 0.4% DOTAP | 0.25% glyceryl | 0.5% sorbitan | 1% aluminum |
| | | trimyristate- | trioleate | hydroxide |
| | | dynasan | (SPAN ® 85) | |
| | | (DYNASAN | 2.0% polysorbate | |
| | | 114 ®) | 80 (TWEEN ® 80 | |
| | | 4.08% squalene | | |
| NP-24 | 0.4% DOTAP | 0.25% glyceryl | 0.25% sorbitan | 1% aluminum |
| | | trimyristate- | trioleate | hydroxide |
| | | dynasan | (SPAN ® 85) | |
| | | (DYNASAN | 2.0% polysorbate | |
| | | 114 ®) | 80 (TWEEN ® | |
| | | 4.08% squalene | 80) | |
| NP-25 | 0.4% DOTAP | 5% squalene | 0.5% sorbitan | 1% aluminum |
| | | | trioleate | hydroxide |
| | | | (SPAN ® 85) | |
| | | | 2.0% polysorbate | |
| | | | 80 (TWEEN ® | |
| | | | 80) | |
| NP-26 | 0.4% DOTAP | 5% squalene | 0.5% sorbitan | 1% aluminum |
| | | | monostearate | hydroxide |
| | | | (SPAN ® 60) | |
| | | | 2% polysorbate | |
| | | | 80 (TWEEN ® | |
| | | | 80) | |
| NP-27 | 0.4% DOTAP | 0.25% glyceryl | 2% sorbitan | 1% aluminum |
| | | trimyristate- | trioleate | hydroxide |
| | | dynasan | (SPAN ® 85) | |
| | | (DYNASAN | 2% polysorbate | |
| | | 114 ®) | 80 (TWEEN ® | |
| | | 4.08% squalene | 80) | |
| NP-28 | 0.5-5.0 mg/ml DOTAP | 0.2-10% (v/v) | 0.01-2.5% (v/v) | |
| | | squalene | polysorbate 80 | |
| | | | (TWEEN ® 80) | |
| NP-29 | 0.4% (w/w) DOTAP | 4.3% (w/w) | 0.5% (w/w) | |
| | | squalene | sorbitan trioleate | |
| | | | (SPAN ® 85) | |
| | | | 0.5% (w/w) | |
| | | | polysorbate 80 | |
| | | | (TWEEN ® 80) | |
| NP-30 | 30 mg/ml DOTAP | 37.5 mg/ml | 37 mg/ml | 10 mM sodium citrate |
| | chloride | squalene | sorbitan | |
| | | | monostearate | |
| | | | (SPAN ® 60) | |
| | | | 37 mg/ml | |
| | | | polysorbate 80 | |
| | | | (TWEEN ® 80) | |
| NP-31 | 30 mg/ml DOTAP | 37.5 mg/ml | 37 mg/ml | 0.4 mg Fe/ml 5 nm |
| | chloride | squalene | sorbitan | oleic acid-coated iron |
| | | | monostearate | oxide nanoparticles |
| | | | (SPAN ® 60) | 10 mM sodium citrate |
| | | | 37 mg/ml | dihydrate |
| | | | polysorbate 80 | |
| | | | (TWEEN ® 80) | |
| NP-32 | 0.8-1.6 mg/ml DOTAP | 4.5% squalene | 0.5% (w/w) | 10 mM sodium citrate |
| | chloride | | sorbitan trioleate | |
| | | | (SPAN 85 ®) | |
| | | | 0.5% (w/w) | |
| | | | polysorbate 80 | |
| | | | (TWEEN ® 80) | |
| NP-33 | 45-55 mol % ionizable | 35-42 mol % | 1.25-1.75 mol % | |
| | cationic lipid | cholesterol | PEG2000-DMG | |
| | 8-12 mol % | | | |
| | distearoylphosphatidyl- | | | |
| | choline (DSPC) | | | |
| NP-34 | 50 mol % D-Lin-MC3- | 38.5% cholesterol | 1.5% PEG-lipid | |
| | DMA (MC3) | | | |
| | 10 mol % | | | |
| | distearoylphosphatidyl- | | | |
| | choline (DSPC) | | | |
| NP-35 | 50 mol % Lipid H | 38.5% cholesterol | 1.5 mol % | |
| | (SM-102) | | PEG2000-DMG | |
| | 10 mol % | | | |
| | distearoylphosphatidyl- | | | |
| | choline (DSPC) | | | |
| NP-36 | 30 mg/ml 1,2- dioleoyl-3. | 3.75% w/v | 37 mg/ml | 10 mM sodium citrate |
| | trimethylammonium- | glyceryl | sorbitan | dihydrate |
| | propane (DOTAP) | trimyristate- | monostearate, | |
| | chloride | dynasan | (2R)-2- | |
| | | (DYNASAN | [(2R,3R,4S)-3,4- | |
| | | 114 ®) | Dihydroxyoxolan- | |
| | | | 2-yl]-2- | |
| | | | hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-37 | 30 mg/ml 1,2- dioleoyl-3- | 3.75% w/v | 37 mg/ml | 0.2 mg Fe/mL or |
| | trimethylammonium- | glyceryl | sorbitan | 0.02% wFe/v of 5 to |
| | propane (DOTAP) | trimyristate- | monostearate, | 15 nm diameter iron |
| | chloride | dynasan | (2R)-2- | oxide nanoparticles |
| | | (DYNASAN | [(2R,3R,4S)-3,4- | 10 mM sodium citrate |
| | | 114 ®) | Dihydroxyoxolan- | dihydrate |
| | | | 2-yl]-2- | |
| | | | hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |

In some embodiments, nanoparticles provided herein comprise: sorbitan monostearate (e.g., SPAN® 60), polysorbate 80 (e.g., TWEEN® 80), DOTAP, squalene, and no solid particles. In some embodiments, nanoparticles provided herein comprise: sorbitan monostearate (e.g., SPAN® 60), polysorbate 80 (e.g., TWEEN® 80), DOTAP, squalene, and iron oxide particles. In some embodiments, nanoparticles provided herein comprise an immune stimulant. In some embodiments, the immune stimulant is squalene. In some embodiments, the immune stimulant is Miglyol 810 or Miglyol 812. Miglyol 810 is a triglyceride ester of saturated caprylic and capric fatty acids and glycerol. Miglyol 812 is a triglyceride ester of saturated coconut/palm kernel oil derived caprylic and capric fatty acids and plant derived glycerol. In some embodiments, the immune stimulant can decrease the total amount of protein produced, but can increase the immune response to a composition provided herein (e.g., when delivered as a vaccine). In some embodiments, the immune stimulant can increase the total amount of protein produced, but can decrease the immune response to a composition provided herein.

Nanoparticles provided herein can be of various average diameters in size. In some embodiments, nanoparticles provided herein have an average diameter (z-average hydrodynamic diameter, measured by dynamic light scattering) ranging from about 20 nanometers (nm) to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle) ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, the inorganic particle (e.g., iron oxide) within the hydrophobic core of the nanoparticle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm. In some embodiments, the ratio of esters and lipids yield a particle size between 30 nm and 200 nm. In some embodiments, the ratio of esters and lipids yield a particle size between 40 nm and 70 nm.

Nanoparticles provided herein may be characterized by the polydispersity index (PDI), which is an indication of their quality with respect to size distribution. In some embodiments, average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, the nanoparticles provided herein comprise a net positive charge. In some embodiments, the nanoparticles provided herein comprise a net positive charge at a temperature of at least about 35 degrees Celsius up to 40 degrees Celsius. In some embodiments, the nanoparticles provided herein comprise a net positive charge when administered to a subject in vivo. Further provided herein are compositions, wherein the compositions comprise: nanoparticles, wherein the nanoparticles comprise: a hydrophobic core comprising lipids in liquid phase at 25 degrees Celsius; and nucleic acids encoding for a protein or an antibody, wherein the nucleic acids are complexed to the nanoparticles to form nucleic acid-nanoparticle complexes, and wherein the nucleic acid-nanoparticle complexes comprise a net positive charge at 37 degrees Celsius.

In some embodiments, the nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, the nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

In some embodiments, nucleic acids provided herein are incorporated, associated with, or complexed a lipid carrier provided herein to form a lipid carrier-nucleic acid complex. The lipid carrier-nucleic acid complex is formed via non-covalent interactions or via reversible covalent interactions.

In some embodiments, nanoparticles provided herein are made by homogenization and ultrasonication techniques. In some embodiments, a nanoparticle provided herein is admixed with a nucleic acid provided herein to form a nanoparticle-nucleic acid complex. In some embodiments, a plurality of nanoparticles provided herein are admixed with a plurality of nucleic acids provided herein to form a plurality of nanoparticle-nucleic acid complexes.

Additional delivery vehicles can be used to deliver a nucleic acid provided herein to a cell or a subject. Non-limiting examples of delivery vehicles include an emulsion, a suspension, a liposome, a micelle, an exosome, an endosome, a virus, a vector, a particle, a nanoparticle, a polymer, microcapsules, recombinant cells, cell culture medium, blood, or serum. Further provided herein are vectors comprising one or more nucleic acids provided herein. In some embodiments, the vector is a viral vector. Exemplary viral vectors include, but are not limited to, lentiviral vectors, retroviral vectors, adeno-associated viral vectors (AAV), adenoviral vectors, herpes simplex viral vectors, alphaviral vectors, flaviviral vectors, rhabdoviral vectors, measles viral vectors, Newcastle disease viral vectors, poxviral vectors, and picomaviral vectors. In some embodiments, polynucleotides encoding an engineered protein provided herein are contained in an AAV viral vector.

### Combination Compositions

Provided herein are compositions comprising a nanoparticle described herein and a nucleic acid encoding for a an interleukin. In some embodiments, the compositions further comprise an innate immune response modulator or a nucleic acid sequence encoding the innate immune response modulator (e.g., a RIG-I agonist or a toll-like receptor agonist). In some embodiments, the compositions further comprise a nucleic acid sequence encoding a cancer-associated protein.

Provided herein are compositions comprising nanoparticles (e.g., NP-30 or NP-1) and a nucleic acid encoding for a protein, an IL-12, a PRR agonist, or a combination thereof. In some embodiments, nucleic acids provided herein are incorporated, associated with, or complexed a lipid carrier provided herein to form a lipid carrier-nucleic acid complex. The lipid carrier-nucleic acid complex is formed via non-covalent interactions or via reversible covalent interactions. The nucleic acids provided herein are in complex with the surface of the lipid nanoparticle provided herein.

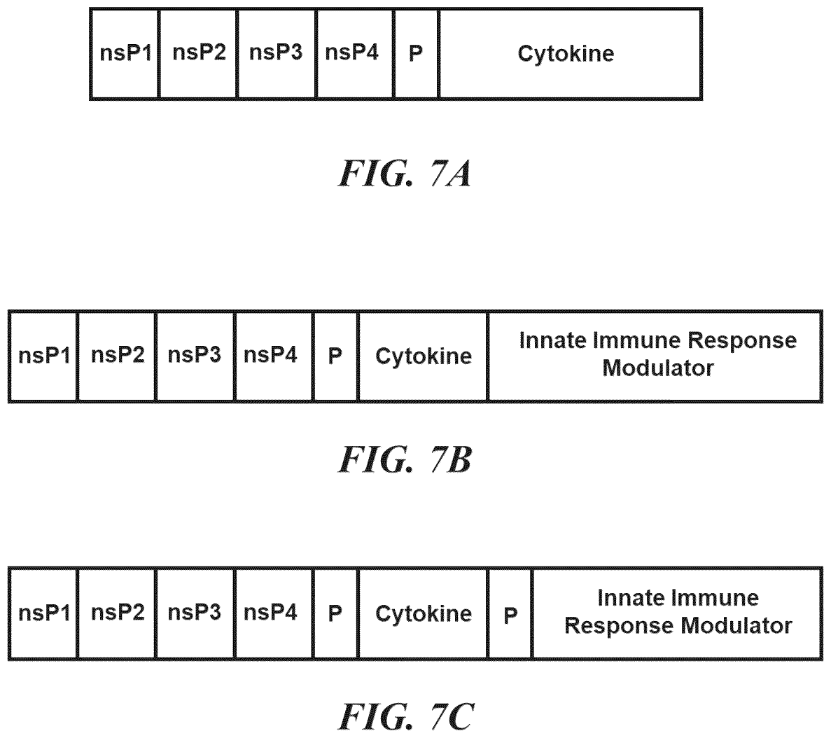

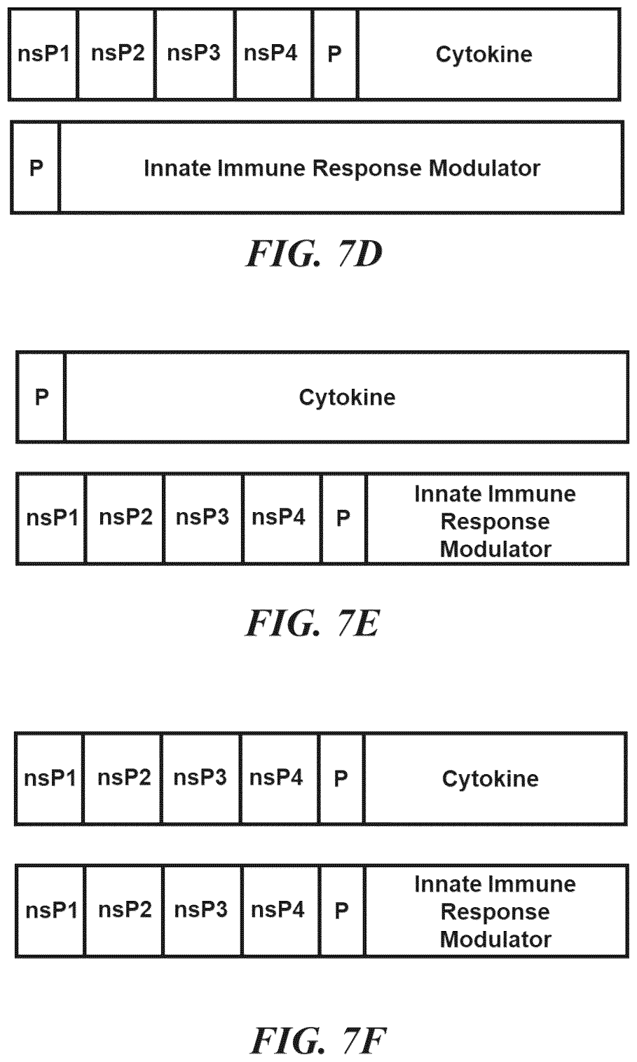

Further provided herein is a nanoemulsion comprising a plurality of nanoparticles provided herein. In some embodiments, the nucleic acid further encodes for an RNA polymerase. In some embodiments the RNA polymerase is a viral RNA polymerase. In some embodiments, the viral RNA polymerase includes a sub-genome from an alphavirus. In some embodiments, the nucleic acid encoding for the RNA polymerase is on the same nucleic acid strand as the nucleic acid sequence encoding for the cytokine (e.g., cis) (FIG. 7A). In some embodiments, the nucleic acid provided herein encodes for a viral RNA polymerase; a cytokine; and an innate immune response modulator on the same strand (FIG. 7B-7C). In some embodiments, the nucleic acid encoding for the RNA polymerase is on a different nucleic acid strand as the nucleic acid sequence encoding for a protein or innate immune response modulator (e.g., trans) (FIGS. 7D-7F). In some embodiments, a first nucleic acid comprises a sequence encoding for an RNA-dependent RNA polymerase and a cytokine (FIG. 7D). In some embodiments, a second nucleic acid comprises a sequence encoding the innate immune response modulator. In some embodiments, a first nucleic acid comprises a sequence encoding for an RNA-dependent RNA polymerase and an innate immune response modulator (FIG. 7E). In some embodiments, a second nucleic acid comprises a sequence encoding for a cytokine. In some embodiments, a composition comprises: (a) a first nucleic acid comprising a sequence encoding for an RNA-dependent RNA polymerase and a cytokine; and (b) a second nucleic acid encoding for an RNA-dependent RNA polymerase and an innate immune response modulator (FIG. 7F). In some embodiments, the nucleic acid encoding for the RNA polymerase is a DNA molecule. In some embodiments, nucleic acid sequences encoding for a cytokine, an innate immune response modulator, a cancer-associated protein, an antigen, a cancer therapeutic antibody, or a functional fragment thereof are DNA or RNA molecules. In some embodiments, the proteins provided herein (e.g., cytokines, cancer-associated proteins, antigens) are encoded by DNA. Nanoparticles for inclusion include, without limitation, any one of NP-1 to NP-31, or any one of NP-1 to NP-37. Nucleic acids for inclusion include, without limitation, comprise a region comprising any one of, or a plurality of, SEQ ID NOS: 16-28, 32, 34-40, 42-45 and/or encodes for an amino acid sequence set forth in any one of SEQ ID NOS: 1-15, 29-31, 41, 46-94. In some instances, the nucleic acids further comprise a region encoding for an RNA polymerase, e.g., a region comprising a sequence of SEQ ID NO: 28.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to the antigen, increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1. In certain embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In one embodiment, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene:DOTAP, hydrophobic surfactant, and hydrophilic surfactant. In some embodiments, the oil-to-surfactant ratio is the molar ratio of squalene:DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-lipid (e.g., cationic lipid) ratio. In some embodiments, the hydrophilic surfactant-to-lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio ranging. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1.

Provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN® 60), polysorbate 80 (e.g., TWEEN® 80), DOTAP, an immune stimulant, and an RNA.

### Thermally Stable, Dried, and Lyophilized Vaccines

Provided herein are dried or lyophilized compositions and vaccines. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

A lyophilized composition is generated by a low temperature dehydration process involving the freezing of the composition, followed by a lowering of pressure, and removal of ice by sublimation. In certain cases, lyophilization also involves the removal of bound water molecules through a desorption process. In some embodiments, compositions and vaccine compositions provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions and vaccine compositions provided herein at higher temperatures (e.g., greater than 4° C.) as compared to the sub-zero temperatures needed for the storage of existing mRNA vaccines. In some embodiments, dried compositions and lyophilized compositions provided herein comprise (a) a lipid carrier, wherein the lipid carrier is a nanoemulsion comprising: (i) a hydrophobic core; (ii) one or more inorganic nanoparticles; (iii) and one or more lipids; (b) one or more nucleic acids; and (c) at least one cryoprotectant. In some embodiments, the cryoprotectant is selected from the group consisting of: sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erythritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least about 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least about 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions and vaccine compositions provided herein are thermally stable. A composition is considered thermally stable when the composition resists the action of heat or cold and maintains its properties, such as the ability to protect a nucleic acid molecule from degradation at given temperature. In some embodiments, compositions and vaccine compositions provided herein are thermally stable at about 25 degrees Celsius (° C.) or standard room temperature. In some embodiments, compositions and vaccine compositions provided herein are thermally stable at about 45° C. In some embodiments, compositions and vaccine compositions provided herein are thermally stable at about −20° C. In some embodiments, compositions and vaccine compositions provided herein are thermally stable at about 2° C. to about 8° C. In some embodiments, compositions and vaccine compositions provided herein are thermally stable at a temperature of at least about −80° C., at least about −20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions and vaccine compositions provided herein are thermally stable for at least about 5 day, at least about 1 week, at least about 2 weeks, at least about 1 month, up to 3 months. In some embodiments, compositions and vaccine compositions provided herein are stored at a temperature of at least about 4° C. up to 37° C. for at least about 5 day, at least about 1 week, at least about 2 weeks, at least about 1 month, up to 3 months. In some embodiments, compositions and vaccine compositions provided herein are stored at a temperature of at least about 20° C. up to 25° C. for at least about 5 day, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### Pharmaceutical Compositions

Provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via a subcutaneous, intradermal, intramuscular, inhalation, intravenous, intraperitoneal, intracranial, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days.

In some embodiments, a single dose of a composition provided herein is administered to a subject. In some embodiments, a composition or pharmaceutical composition provided herein is administered to the subject by two doses. In some embodiments, a second dose of a composition or pharmaceutical composition provided herein is administered about 28 days or 56 days after the first dose. In some embodiments, a first dose is administered, and a second dose is administered about 14 days later, or about 21 days later, or about 28 days later, or about 35 days later, or about 42 days later, or about 49 days later, or about 56 days later, or about 63 days later, or about 70 days later, or about 77 days later, or about 84 days later. In some embodiments, the second dose is administered about 10-90 days following administration of the first dose, or about 15-85 days following administration of the first dose, or about 20-80 days following administration of the first dose, or about 25-75 days following administration of the first dose, or about 30-70 days following administration of the first dose, or about 35-65 days following administration of the first dose, or about 40-60 days following administration of the first dose.

In some embodiments, a third dose of a composition or pharmaceutical composition provided herein is administered to a subject. In some embodiments, the third dose is administered about 1 month following administration of the second dose, about 2 months following administration of the second dose, about 3 months following administration of the second dose, about 4 months following administration of the second dose, about 5 months following administration of the second dose, about 6 months following administration of the second dose, about 7 months following administration of the second dose, about 8 months following administration of the second dose, about 9 months following administration of the second dose, about 10 months following administration of the second dose, about 11 months following administration of the second dose, about 12 months following administration of the second dose, about 13 months following administration of the second dose, about 14 months following administration of the second dose, about 15 months following administration of the second dose, about 16 months following administration of the second dose, about 17 months following administration of the second dose, or about 18 months following administration of the second dose.

### Therapeutic Applications

Provided herein are methods of treating or preventing a disease in a subject. Further provided herein are methods of modulating an immune response in a subject. Further provided herein are methods of inducing an innate immune response in a subject.

In some embodiments, the immune response may comprise at least one of production of one or a plurality of cytokines or chemokines. In some embodiments, the cytokine or chemokine is selected from the group consisting of those listed in Table 1, IL-6, IL-12, tumor necrosis factor alpha (TNF-α), interferon-gamma (IFN-γ), MIP-1α, MIP-1β, RANTES, CCL2, CCL4, CCL5, CXCL1, and CXCL5. In some embodiments, the immune response comprises activation or proliferation of a population of lymphocytes in a subject. In some embodiments, the population of lymphocytes comprises: NK cells, CD8+ T cells, CD4+ T cells, T-bet positive (Tbet⁺) T cells, tumor infiltrating lymphocytes, or any combination thereof. In some embodiments, the immune response comprises a lymphocyte response that is selected from a memory T cell response, a memory B cell response, an effector T cell response, a cytotoxic T cell response, memory CD8 T cell expansion or response, type 1 helper T cell expansion or response, and an effector B cell response.

In some embodiments, compositions described herein are used for the treatment of cancer. In some embodiments, the subject has, is suspected of having, or is at risk of developing cancer. In some embodiments, the subject has a solid tumor or a blood cancer. In some embodiments, the solid tumor is a carcinoma, a melanoma, or a sarcoma. In some embodiments, the subject has a metastatic tumor. In some embodiments, the blood cancer is lymphoma or leukemia. In some embodiments, a composition provided herein is administered to a subject, thereby reducing tumor volume in the subject.

In some embodiments, compositions provided herein are used for prophylactically immunizing a subject for a cancer. In some embodiments, compositions described herein are used for prophylactically immunizing a subject for a skin cancer or a breast cancer.

In some embodiments, the method for treatment of cancer comprises administration of a composition provided herein and radiation. In some embodiments, the composition comprises a high atomic number (Z) element. In some aspects, the high-Z element of the embodiments is gold, silver, iodine, gallium, barium, iron, gadolinium, platinum, hafnium, bismuth or combinations thereof. In some embodiments, the composition comprises a nanoparticle lipid carrier comprising an inorganic particle. In some embodiments, the inorganic nanoparticle comprises iron oxide, optionally superparamagnetic iron oxide. In some embodiments, the inorganic particle comprises aluminum oxide, aluminum oxyhydroxide, iron oxide, titanium dioxide, or silicon dioxide. In some embodiments, the composition comprises a superparamagnetic agent. In some embodiments, the superparamagnetic agent comprises a metal oxides or sulfides which experience a magnetic domain. In some embodiments, the superparamagnetic agent comprises pure iron, magnetite, y-Fe₂O3, Fe₃O₄, manganese ferrite, cobalt ferrite and nickel ferrite. In some embodiments, the radiation therapy comprises low energy superficial kilovoltage, orthovoltage X-ray, high energy megavoltage (MV) photons, electron beam therapy (Linac), cobalt therapy, or brachytherapy. In some embodiments, the radiation therapy comprises administration of an X-ray, electron, gamma-ray, alpha or beta rays, or radioactive source (e.g., Au, CO, Cesium, and Iridium) localized into tumor tissue. In some embodiments the radiation is applied to localized superficial skin cancers, skin cancer with deep penetration, large or thick lesions, or critical sites of a subject. Further provided herein are methods where the radiation dose is lower than the standard treatment dose due to the activity of the nanoparticle. Further provided herein are methods where the radiation is delivered by administering a radioactive isotope to the subject. Further provided herein is where the radioactive isotope is yttrium-90, or lutetium-177, or iodine-131, or samarium-153, or phosphorus-32. Further provided herein is where the isotope is delivered via a therapeutic. Further provided herein is where the isotope is bound to a monoclonal antibody. In some embodiments, the radiation is applied to a dermatological condition. In some embodiments, the dermatological condition is BCC, SCC, Bowen's disease, Erythroplasia, Angiosarcoma, Keratoacanthoma, Melanoma, Merkel cell carcinoma, Cutaneous lymphoma, Kaposi's sarcoma, or Fibrosarcoma. In some embodiments the dose is up to 35 Gy, up to 55 Gy, or from about 35 to about 55 Gy. In some embodiments, the radiotherapy comprises ionizing radiation administered at one time or as fractions over a period of time. In some embodiments, the schedule is over about 1 week to about 6 weeks. In some embodiments, the modality of irradiation comprises Grenz Rays, contact therapy, short source surface distance, superficial therapy, or orthovoltage therapy. In some embodiments the ionizing radiation is about 10-20 kV, 40-50 kV, 50-150 kV or 150-300 kV. In some embodiments, the ionizing radiation is at one or more energy levels from 1 kV to 10 MV photons or up to 300 MeV heavy ions. In some embodiments the treatment depth is <1 mm, 1-2 mm, >5 mm, or >5 mm and <2 cm. In some embodiments, the administration is to the brain, lung, liver, spleen, kidney, lymph node, small intestine, pancreas, blood, colon, stomach, breast, endometrium, prostate, testicle, ovary, skin, head and neck, esophagus, lymphatic, bone marrow or bone cancer cells.

In some embodiments, compositions provided herein are used for the treatment of an infection. In some embodiments, the subject is at risk of developing an infectious disease or disorder. In some embodiments, the subject has contracted an infectious disease by way of contact with another infected subject. In some embodiments, the subject has contracted an infectious disease from contaminated drinking water. In some embodiments, the subject has contracted the infectious disease from a different species carrying the microorganism. In some embodiments, the subject is a mammal. In some embodiments, the mammal is a human. In some embodiments, the infection is a viral infection, a bacterial infection, a parasitic infection, a fungal infection, or a yeast infection. In some embodiments, the subject has, is suspected of having, or is at risk of developing a viral infection. Non-limiting examples of infectious microorganisms and infectious agents that can be treated with the compositions provided herein include but are not limited to: viruses such as adenoviruses, herpes simplex type 1 virus, herpes simplex type 2 virus, encephalitis virus, papillomavirus, varicella-zoster virus (VZV), Epstein-Barr virus (EBV), human cytomegalovirus (CMV), Chikungunya virus, human herpes virus type 8, human papillomavirus (HPV), BK virus, JC virus, smallpox, polio virus, hepatitis B virus, human bocavirus, parvovirus B19, human astrovirus, Norwalk virus, coxsackievirus, hepatitis A virus, poliovirus, rhinovirus, Severe acute respiratory syndrome (SARS) virus, SARS-CoV-2, yellow fever virus, Dengue virus, West Nile virus, rubella virus, hepatitis E virus, human immunodeficiency virus (HIV), influenza virus (influenza A or influenza B), Guanarito virus, Junin virus, Lassa virus, Machupo virus, Sabiá virus, Crimean-Congo hemorrhagic fever virus, Ebola virus, Marburg virus, measles virus, mumps virus, Parainfluenza virus, respiratory syncytial virus (RSV), human metapneumovirus, Hendra virus, Nipah virus, rabies virus, hepatitis D, rotavirus, orbivirus, coltivirus, banna virus, zika virus, hanta virus, West Nile virus, Middle East Respiratory Syndrome (MERS) coronavirus, Japanese encephalitis virus, and Eastern equine encephalitis; bacteria such as Acetobacter, Acinetobacter, Actinomyces, Agrobacterium, Anaplasma, Azorhizobia, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rothia, Salmonella, Shigella, Staphylococcus, Stenotrophomonas, Streptococcus, Streptococcus pneumoniae, Treponema, Vibrio, Walbachia, and Yersinia; fungi such as Aspergillus, Saccharomyces, Cryptococcus, Coccidioides, Neurospora, Histoplasma, Blastomyces; parasites such as Babesia sp., Cryptosporidium sp., Plasmodium sp., Toxoplasma sp. Plasmodium sp., Plasmodium falciparum, Plasmodium vivax, Cryptosporidium parvum, Cryptosporidium hominis, Eimeria sp., Eimeria tenella, Theileria sp., Theileria parva, Toxoplasma sp. Toxoplasma gondii, Trypanosoma brucei subspecies, Trypanosoma cruzi, Leishmania sp., and Leishmania major; and yeast such as Candida.

In some embodiments, compositions provided herein can be used to treat an inflammatory disease, disorder, or condition. In some embodiments, the subject has, is suspected of having or is at risk for developing an inflammatory disease, disorder, or condition. In some embodiments, the inflammatory disease is psoriasis, arthritis, an autoimmune disease, pain, allergy, asthma, or a neurological disease. Exemplary autoimmune diseases include but are not limited to: multiple sclerosis, rheumatoid arthritis, Hashimoto thyroiditis, type I diabetes mellitus (Juvenile onset diabetes) and autoimmune uveoretinitis, myasthenia gravis, systemic lupus erythematosus (or SLE), Graves' disease, autoimmune hemolytic anemia, autoimmune thrombocytopenia, autoimmune asthma, cryoglobulinemia, thrombic thrombocytopenic purpura, primary biliary sclerosis and pernicious anemia.

### Kits

Provided herein is a kit comprising a composition provided herein, a pharmaceutical composition provided herein; and optionally, a delivery system for administration to a subject. Kits described herein may comprise lyophilized reagents and, optionally, a reagent for hydration. Kits described herein may also comprise non-lyophilized reagents. In some embodiments, the kit comprises two or more separate units comprising the lipid carrier and the nucleic acid, respectively.

In some embodiments, the kit comprises a unit that comprises the lipid carrier and the nucleic acid. In some embodiments, the kit further comprises a unit comprising a reagent for hydration of the dried composition. In some embodiments, the reagent for hydration comprises water.

In some embodiments, a composition provided herein is prepared in a single container for administration. In some embodiments, a composition provided herein is prepared in two containers for administration, separating one or more nucleic acids provided herein from the nanoparticle carrier or separating nanoparticles from one another. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

Provided herein is kit, wherein the kit comprises: a first container comprising: a lipid carrier provided herein; and a nucleic acid encoding for a cytokine or a functional variant thereof; and a second container comprising: a lipid carrier provided herein; and a nucleic acid encoding an innate immune response modulator or a functional variant thereof. Further provided herein is kit, wherein the kit comprises: a first container comprising: a lipid carrier provided herein; and a second container comprising: a lipid carrier provided herein; and a nucleic acid encoding for an innate immune response modulator or a functional variant thereof. Further provided herein is kit, wherein the kit comprises: a first container comprising: a lipid carrier provided herein; and a second container comprising: a lipid carrier provided herein; and a nucleic acid encoding for a cytokine or a functional variant thereof. Further provided herein is kit, wherein the kit comprises: a first container comprising: a lipid carrier provided herein; and a second container comprising: a nucleic acid encoding an innate immune response modulator or a functional variant thereof; and a cytokine provided herein or a functional variant thereof.

In some embodiments, the kit comprises: (a) a lipid carrier, wherein the lipid carrier is a nanoemulsion comprising a hydrophobic core, one or more lipids, and one or more surfactants; and (b) at least one nucleic acid sequence, wherein the nucleic acid comprises a sequence encoding for a cytokine; and optionally, an innate immune response modulator. In some embodiments, the kit further comprises one or more surfactants.

Kits are useful for providing efficient and or large-scale manufacturability of composition provided herein.

### Exemplary Embodiments

Provided herein are compositions, wherein the compositions comprise: a lipid carrier, wherein a surface of the lipid carrier comprises cationic lipids; and a nucleic acid encoding for (i) a RNA-dependent RNA polymerase and (ii) an IL-12, a lipid carrier, wherein a surface of the lipid carrier comprises cationic lipids; and a nucleic acid encoding for: (i) an RNA-dependent RNA polymerase; and (ii) an IL-12, wherein the surface of the lipid carrier and the nucleic acid form a lipid carrier-nucleic acid complex. Further provided herein are compositions, wherein the IL-12 is a modified IL-12. Further provided herein are compositions, wherein the modified IL-12 comprises a linker between a p35 subunit and a p40 subunit. Further provided herein are compositions, wherein the compositions further comprise an innate immune response modulator. Further provided herein are compositions, wherein the innate immune response modulator comprises a pattern recognition receptor (PRR) agonist or a nucleic acid encoding for a PRR agonist. Further provided herein are compositions, wherein the PRR agonist is a RIG-I-like receptor (RLR) agonist. Further provided herein are compositions, wherein the RLR agonist is RIG-I. Further provided herein are compositions, wherein the PRR agonist is a TLR3 agonist or a TLR4 agonist. Further provided herein are compositions, wherein the TLR4 agonist is glucopyranosyl lipid A (GLA), monophosphoryl lipid A (MPLA), lipopolysaccharide (LPS), or a derivative thereof. Further provided herein are compositions, wherein the linker comprises 14 to 18 amino acids. Further provided herein are compositions, wherein the RNA-dependent RNA polymerase includes a sub-genome of an alphavirus. Further provided herein are compositions, wherein the nucleic acid comprises an RNA sequence of SEQ ID NO: 28. Further provided herein are compositions, wherein the lipid carrier comprises a cationic lipid and a hydrophobic core. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,127,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Provided herein are compositions, wherein the compositions comprise: a lipid carrier, wherein a surface of the lipid carrier comprises cationic lipids; and a nucleic acid encoding for (i) a RNA-dependent RNA polymerase and (ii) a cytokine; and an innate immune response modulator. Further provided herein are compositions, wherein the cytokine comprises IL-12, IFN-gamma (IFNγ), IL-2, IL-15, IL-18, IL-21, IL-23, or a functional variant thereof. Further provided herein are compositions, wherein the cytokine comprises an amino acid sequence of one of: SEQ ID NOS: 1 to 15, or a functional fragment thereof. Further provided herein are compositions, wherein the cytokine comprises an amino acid sequence of SEQ ID NO: 1, SEQ ID NO: 2, SEQ ID NO: 3, SEQ ID NO: 4, SEQ ID NO: 5, SEQ ID NO: 6, and any combination thereof. Further provided herein are compositions, wherein the nucleic acid is an RNA or a DNA. Further provided herein are compositions, wherein the nucleic acid encodes double-stranded RNA. Further provided herein are compositions, wherein the nucleic acid encodes single-stranded RNA. Further provided herein are compositions, wherein the nucleic acid comprises a sequence of: SEQ ID NO: 16, SEQ ID NO: 42, SEQ ID NO: 43, or SEQ ID NO: 45. Further provided herein are compositions, wherein the nucleic acid sequence comprises SEQ ID NO: 28. Further provided herein are compositions, wherein the nucleic acid sequence comprises SEQ ID NO: 32 or SEQ ID NO: 43. Further provided herein are compositions, wherein the innate immune response modulator comprises a pattern recognition receptor (PRR) agonist or a nucleic acid encoding for a pattern recognition receptor (PRR) agonist. Further provided herein are compositions, wherein the PRR agonist is a RIG-I-like receptor (RLR) agonist. Further provided herein are compositions, wherein the RLR agonist is RIG-I. Further provided herein are compositions, wherein the PRR agonist is a TLR3 agonist or a TLR4 agonist. Further provided herein are compositions, wherein the innate immune response modulator comprises a TLR4 agonist. Further provided herein are compositions, wherein the TLR3 agonist is a nucleic acid comprising one of: SEQ ID NOS: 17-27. Further provided herein are compositions, wherein the lipid carrier comprises a cationic lipid and a hydrophobic core. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 micrograms (μg) to about 200 μg. Further provided herein are compositions, wherein the nucleic acid is present in an amount of up to about 25 nanograms (ng), about 50 ng, about 75 ng, about 100 ng, about 150 ng, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Further provided herein are compositions, wherein the compositions comprise: a lipid carrier; and a nucleic acid encoding for: (i) a RNA-dependent RNA polymerase; (ii) a cytokine; and (iii) an innate immune response modulator. Further provided herein are compositions, wherein the cytokine comprises IL-12, IFN-gamma (IFNγ), IL-2, IL-15, IL-18, IL-21, IL-23, or a functional variant thereof. Further provided herein are compositions, wherein the cytokine comprises an amino acid sequence of one of: SEQ ID NOS: 1 to 15, or a functional fragment thereof. Further provided herein are compositions, wherein the cytokine comprises an amino acid sequence of SEQ ID NO: 1, SEQ ID NO: 2, SEQ ID NO: 3, SEQ ID NO: 4, SEQ ID NO: 5, SEQ ID NO: 6, and any combination thereof. Further provided herein are compositions, wherein the composition further comprises an innate immune response modulator. Further provided herein are compositions, wherein the innate immune response modulator is RIG-I. Further provided herein are compositions, wherein the innate immune response modulator is a TLR3 agonist or a TLR4 agonist. Further provided herein are compositions, wherein the nucleic acid is an RNA or a DNA. Further provided herein are compositions, wherein the nucleic acid encodes double-stranded RNA. Further provided herein are compositions, wherein the nucleic acid encodes single-stranded RNA. Further provided herein are compositions, wherein the nucleic acid comprises a sequence of SEQ ID NO: 16, SEQ ID NO: 42, or SEQ ID NO: 45. Further provided herein are compositions, wherein the nucleic acid sequence comprises SEQ ID NO: 28. Further provided herein are compositions, wherein the nucleic acid sequence comprises SEQ ID NO: 32 or SEQ ID NO: 43. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the lipid carrier comprises a hydrophobic core. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Provided herein are compositions, wherein the compositions comprise: a lipid carrier, wherein the lipid carrier comprises: a cationic lipid; and a hydrophobic core, wherein the hydrophobic core comprises an inorganic nanoparticle; and a nucleic acid encoding for: (i) a RNA-dependent RNA polymerase and (ii) a cytokine. Further provided herein are compositions, wherein the cytokine comprises IL-12, IFN-gamma (IFNγ), IL-2, IL-15, IL-18, IL-21, IL-23, or a functional variant thereof. Further provided herein are compositions, wherein the cytokine comprises an amino acid sequence of one of: SEQ ID NOS: 1 to 15, or a functional fragment thereof. Further provided herein are compositions, wherein the cytokine comprises an amino acid sequence of SEQ ID NO: 1, SEQ ID NO: 2, SEQ ID NO: 3, SEQ ID NO: 4, SEQ ID NO: 5, SEQ ID NO: 6, or any combination thereof. Further provided herein are compositions, wherein the nucleic acid is an RNA or a DNA. Further provided herein are compositions, wherein the nucleic acid encodes double-stranded RNA. Further provided herein are compositions, wherein the nucleic acid encodes single-stranded RNA. Further provided herein are compositions, wherein the nucleic acid comprises a sequence of: SEQ ID NO: 16, SEQ ID NO: 42, or a SEQ ID NO: 45. Further provided herein are compositions, wherein the nucleic acid sequence comprises SEQ ID NO: 28 (VEEV). Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-TH-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the lipid carrier comprises a hydrophobic core. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Provided herein are compositions, wherein the compositions comprise: a lipid carrier, wherein a surface of the lipid carrier comprises cationic lipids; and a nucleic acid encoding for (i) a RNA-dependent RNA polymerase and (ii) a modified IL-12, wherein the modified IL-12 comprises a linker between a p35 subunit and a p40 subunit. Further provided herein are compositions, wherein the compositions further comprise an innate immune response modulator. Further provided herein are compositions, wherein the innate immune response modulator comprises a pattern recognition receptor (PRR) agonist or a nucleic acid encoding for a PRR agonist. Further provided herein are compositions, wherein the PRR agonist is a RIG-I-like receptor (RLR) agonist. Further provided herein are compositions, wherein the RLR agonist is RIG-I. Further provided herein are compositions, wherein the PRR agonist is a TLR3 agonist or a TLR4 agonist. Further provided herein are compositions, wherein the TLR4 agonist is glucopyranosyl lipid A (GLA), monophosphoryl lipid A (MPLA), lipopolysaccharide (LPS), or a derivative thereof. Further provided herein are compositions, wherein the linker comprises 14 to 18 amino acids. Further provided herein are compositions, wherein the RNA-dependent RNA polymerase includes a sub-genome of an alphavirus. Further provided herein are compositions, wherein the nucleic acid comprises an RNA sequence of SEQ ID NO: 28. Further provided herein are compositions, wherein the lipid carrier comprises a cationic lipid and a hydrophobic core. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-TH-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Provided herein are compositions, wherein the compositions comprise: a lipid carrier; and a nucleic acid encoding for (i) a RNA-dependent RNA polymerase and (ii) a cytokine; and an innate immune response modulator, wherein the innate immune response modulator is a nucleic acid, wherein the nucleic acid comprises a region coding a sequence at least 85% identical to SEQ ID NO: 17. Further provided herein are compositions, wherein the sequence is at least 90% identical to SEQ ID NO: 17. Further provided herein are compositions, wherein the sequence is at least 95% identical to SEQ ID NO: 17. Further provided herein are compositions, wherein the sequence is SEQ ID NO: 17. Further provided herein are compositions, wherein the sequence is at least 90% identical to SEQ ID NO: 18. Further provided herein are compositions, wherein the sequence is at least 95% identical to SEQ ID NO: 18. Further provided herein are compositions, wherein the sequence is SEQ ID NO: 18. Further provided herein are compositions, wherein the RNA-dependent RNA polymerase includes a sub-genome of an alphavirus. Further provided herein are compositions, wherein the nucleic acid comprises a sequence that is at least 85% identical to the RNA sequence of SEQ ID NO: 16, SEQ ID NO: 42, SEQ ID NO: 43, or SEQ ID NO: 45. Further provided herein are compositions, wherein the nucleic acid is present in an amount of up to about 10 μg. Further provided herein are compositions, wherein the nucleic acid is present in an amount of about 0.05, 0.1, 0.2, 0.5, 1, 5, 10, 12.5, 15, 20, 25, 50, 75, 100, 125, 150 or 200 μg. Further provided herein are compositions, wherein the lipid carrier is up to 100 nm in diameter. Further provided herein are compositions, wherein the lipid carrier is 40 to 80 nm in diameter. Further provided herein are compositions, wherein the lipid carrier comprises a membrane. Further provided herein are compositions, wherein the lipid carrier comprises a hydrophilic surface. Further provided herein are compositions, wherein the hydrophilic surface comprises a cationic lipid. Further provided herein are compositions, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions, wherein the ratio of the cationic lipid to the nucleic acid is up to about 40:1. Further provided herein are compositions, wherein the ratio of the cationic lipid to the nucleic acid is up to about 8:1. Further provided herein are compositions, wherein the ratio of the cationic lipid to the nucleic acid is 25:1 to 100:1. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-TH-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Further provided herein are compositions, wherein the compositions comprise: a lipid carrier; and a nucleic acid encoding for: a RNA-dependent RNA polymerase; a cytokine; and an innate immune modulator coding a sequence at least 85% identical to SEQ ID NO: 17. Further provided herein are compositions, wherein the cytokine comprises IL-12, IFN-gamma (IFNγ), IL-2, IL-15, IL-18, IL-21, IL-23, or a functional variant thereof. Further provided herein are compositions, wherein the cytokine comprises an amino acid sequence of one of SEQ ID NOS: 1 to 13, or a functional fragment thereof. Further provided herein are compositions, wherein the cytokine comprises an amino acid sequence of SEQ ID NO: 1, SEQ ID NO: 2, SEQ ID NO: 3, SEQ ID NO: 4, SEQ ID NO: 5, or SEQ ID NO: 6. Further provided herein are compositions, wherein the sequence is at least 90% identical to SEQ ID NO: 17. Further provided herein are compositions, wherein the sequence is at least 95% identical to SEQ ID NO: 17. Further provided herein are compositions, wherein the sequence is SEQ ID NO: 17. Further provided herein are compositions, wherein the RNA-dependent RNA polymerase includes a sub-genome of an alphavirus. Further provided herein are compositions, wherein the nucleic acid comprises a region coding SEQ ID NO: 28. Further provided herein are compositions, wherein the nucleic acid is present in an amount of up to about 10 μg. Further provided herein are compositions, wherein the nucleic acid is present in an amount of about 0.05, 0.1, 0.2, 0.5, 1, 5, 10, 12.5, 15, 20, 25, 50, 75, 100, 125, 150 or 200 μg. Further provided herein are compositions, wherein the lipid carrier is up to 100 nm in diameter. Further provided herein are compositions, wherein the lipid carrier is 40 to 80 nm in diameter. Further provided herein are compositions, wherein the lipid carrier comprises a membrane. Further provided herein are compositions, wherein the lipid carrier comprises a hydrophilic surface. Further provided herein are compositions, wherein the hydrophilic surface comprises a cationic lipid. Further provided herein are compositions, wherein a ratio of amount of the cationic lipid to amount of the nucleic acid is up to about 100:1, and wherein the amount of the cationic lipid is measured based on positively charged nitrogen molar amount and the amount of the nucleic acid is measured based on negatively charged phosphate molar amount. Further provided herein are compositions, wherein the ratio of the cationic lipid to the nucleic acid is up to about 40:1. Further provided herein are compositions, wherein the ratio of the cationic lipid to the nucleic acid is up to about 8:1. Further provided herein are compositions, wherein the ratio of the cationic lipid to the nucleic acid is 25:1 to 100:1. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl)bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-TH-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase at 25 degrees Celsius. Further provided herein are compositions, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the lipid carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the lipid carrier comprises a cationic lipid and an oil. Further provided herein are compositions, wherein the lipid carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: (a) a phosphate-terminated lipid; and (b) a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 μg to about 200 μg. Further provided herein are compositions, wherein the nucleic acid is present in an amount of up to about 25, about 50, about 75, about 100, about 150, or about 175 ng. Further provided herein are compositions, wherein the nucleic acid is present in an amount of up to about 1 μg. Further provided herein are compositions, wherein the nucleic acid is present in an amount of about 0.05, about 0.1, about 0.2, about 0.5, about 1, about 5, about 10, about 12.5, about 15, about 25, about 40, about 50, about 100, about 150, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid carrier is in an aqueous solution.

Provided herein are methods for modulating an immune response in a subject, wherein the methods comprise: administering to the subject a composition provided herein. Further provided herein are methods for modulating an immune response, wherein the administering is intranasal, subcutaneous, intravenous, via inhalation, intramuscular, intratumoral, peritumoral, or intradermal. Further provided herein are methods for modulating an immune response, wherein the administering is systemic. Further provided herein are methods for modulating an immune response, wherein the subject has cancer. Further provided herein are methods for modulating an immune response, wherein the subject has, is at risk of developing, or is suspected of having an infection. Further provided herein are methods for modulating an immune response, wherein the methods induce an innate immune response in a subject.

Provided herein are methods for treating cancer in a subject, wherein the methods comprise: administering to the subject a composition provided herein, thereby treating the cancer. In some embodiments, the administering is intratumoral. Further provided herein are methods for treating cancer in a subject, wherein the administering is intranasal, subcutaneous, intravenous, via inhalation, intramuscular, intratumoral, peritumoral, vaginal, intrathecal, or intradermal. Further provided herein are methods for treating cancer in a subject, wherein the administering is local or systemic. Further provided herein are methods for treating cancer in a subject, wherein the cancer is a solid cancer or a hematopoietic cancer. Further provided herein are methods for treating cancer in a subject, wherein the cancer is a solid cancer, and wherein the solid cancer is a skin cancer (e.g., melanoma), a breast cancer, a colon cancer, lung cancer, a liver cancer, a head and neck cancer, a pancreatic cancer, a prostate cancer, an ovarian cancer, or a uterine cancer. In some embodiments, the skin cancer is a basal cell cancer, a melanoma, a Merkel cell cancer, a squamous cell carcinoma, a cutaneous lymphoma, a Kaposi sarcoma, or a skin adnexal cancer. In some embodiments, the lung cancer is a non-small cell lung cancer (NSCLC) or a small cell lung cancer (SCLC). In some embodiments, the NSCLC is an adenocarcinoma, a squamous cell carcinoma, a large cell carcinoma, an adenosquamous carcinoma, or a sarcomatoid carcinoma. In some embodiments, the pancreatic cancer is a pancreatic adenocarcinoma or a pancreatic exocrine cancer. In some embodiments, the pancreatic cancer is a pancreatic neuroendocrine cancer, an islet cell cancer, or a pancreatic endocrine cancer. In some embodiments, the breast cancer is an invasive ductal carcinoma, an invasive lobular cancer, a non-invasive breast cancer, or a cancerous phyllodes. In some embodiments, the breast cancer is a triple negative breast cancer (e.g., does not overexpress estrogen, progesterone, and the HER-2/neu gene).

In some embodiments, the solid tumor comprises an oncogenic driver mutation. Non-limiting examples of oncogenic driver genes/biomarkers and their associated cancer type include: breast cancer: BRCA1, BRCA2, TP53, TTN, FLG, OBSCN, ERBB2, GATA3, FGFR1, CCND1, PIK3CA, CACNA1C, ARHGAP35, ARID5B, BIRC6, CDH1, CTCF, DSPP, HDAC9, KDM5B, MAST1, MEF2A, NCOR2, SETD1A, SXL2, RID1A, CTNND1, NUP107, CHD8, FANCI, CHD9, CTCF, KEAP1, PCDH18, LAMA2, HDAC9, ARFGEF1, MLLT4, NRK, FOXO3, CDKN2A, MAP3K1, GPS2, ROCK2, RYR2, PGR, STAT6, PIK3CD, CTCF, CDH1, GATA3, AKT1; gastric cancers: ADCY3, BCL6B, CACNA1C, FRMD4A, NID1, ROCK2; pancreatic cancer: ARHGAP35, CACNA1C, GRIA3, PDAC, PALB2, KRAS, CDKN2A, TP53, and SMAD4; lung cancer: EGFR, MET, KRAS, ALK, ALK L1196M, ALK C1156Y, EML4-ALK, ERBB3, ERBB4, VEGFR, NBPF12, NTRK, ROCK2, RYR2, SCAF11, SDK2, STAT6; prostate cancer: SLC45A3, DNAH12, DSPP, KRAS, PCDH11X, ovarian cancer: DNAH14, PGR, PIK3CD, TTN; colon cancer: LAMA1, PIK3CD, TTN; bladder: RYR2; skin: BRAF V600, NRAS, NRAS Q61L/R, GNAQ, GNA11, AC1, PPP6C, RAC1, PPP6C, STK19.

Provided herein are methods for treating cancer in a subject, wherein the methods provide for reduction in size and/or volume of the cancer. Further provided herein are methods for treating cancer in a subject, wherein the methods provide for reduction of tumor metastasis. Further provided herein are methods for treating cancer in a subject, the method further comprises administering radiation to the subject. Further provided herein are methods for treating cancer in a subject, wherein administering the radiation comprises administering low energy superficial kilovoltage, orthovoltage X-ray, high energy megavoltage (MV) photons, electron beam therapy (Linac), cobalt therapy, or brachytherapy. Further provided herein are methods for treating cancer in a subject, administering the radiation comprises administering an X-ray, electron, gamma-ray, alpha ray or beta ray. Further provided herein are methods for treating cancer in a subject, wherein radiation is delivered by administering a radioactive isotope to the subject. Further provided herein are methods for treating cancer in a subject, wherein the radioactive isotope is delivered via a therapeutic. Further provided herein are methods for treating cancer in a subject, wherein the radiation is administered to localized superficial skin cancers, skin cancer with deep penetration, large or thick lesions, or critical sites of the subject.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Techniques and Materials for Production of Lipid Carriers

The following materials were used in the manufacturing of lipid-inorganic nanoparticles (i.e., lipid carriers). The compositions, kits and methods described herein are not limited to the techniques or materials described herein.

Iron oxide nanoparticles at 25 mg Fe/ml in chloroform and of various average diameters (5, 10, 15, 20, 25 and 30 nm) were purchased from Ocean Nanotech (San Diego, CA, USA). Squalene and SPAN® 60 (sorbitan monostearate) were purchased from Millipore Sigma. TWEEN® 80 (polyethylene glycol sorbitan monooleate) and sodium citrate dihydrate were purchased from Fisher Chemical. The chloride salt of the cationic lipid 1,2-dioleoyl-3-trimethylammonium-propane (DOTAP chloride) was purchased from Corden Pharma. Ultrapure water (18.2 mega ohm-centimeter (MOhm-cm) resistivity) was obtained from a Milli-Q water purification system (Millipore Sigma).

The lipid carrier comprises squalene, sorbitan monostearate (e.g., SPAN® 60), polysorbate 80 (e.g., TWEEN® 80), DOTAP chloride, iron oxide nanoparticles and sodium citrate dihydrate. In general, to iron oxide nanoparticles with a number-weighted average diameter of 5 nm, chloroform was added. Chloroform was allowed to evaporate in a fume hood leaving behind a dry coating of iron oxide nanoparticles. To the iron oxide nanoparticles, SPAN® 60, squalene, and DOTAP chloride were added to prepare the “oil” phase.

The oil phase was sonicated 30 minutes in a water bath pre-heated to 60° C. Separately, in a 1 liter glass bottle, the “aqueous” phase was prepared by adding TWEEN® 80 to sodium citrate dihydrate solution prepared with Milli-Q water.

The aqueous phase was stirred for 30 minutes to allow complete dissolution of TWEEN® 80. After complete dissolution of TWEEN® 80, the aqueous phase was transferred to a beaker and incubated in a water bath pre-heated to 60° C. To the heated oil phase, the pre-heated aqueous phase was added.

The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi.

The fluid was passaged until the z-average hydrodynamic diameter, measured by dynamic light scattering (Malvern Zetasizer Nano S), was 59 nm with a 0.2 polydispersity index.

The microfluidized lipid carrier sample was terminally filtered with a 200 nm pore-size polyethersulfone (PES) syringe filter.

### Example 2: Techniques and Materials for Producing Lipid Carriers with Various Iron Oxide Nanoparticles within the Hydrophobic Core

The following materials were used in the manufacturing of lipid-inorganic nanoparticles (i.e., lipid carriers). The compositions, kits and methods described herein are not limited to the techniques or materials describe herein.

Iron oxide nanoparticles at 25 mg Fe/ml in chloroform and of various average diameters (5, 10, 15, 20, 25 and 30 nm) were purchased from Ocean Nanotech (San Diego, CA). Squalene and SPAN® 60 (sorbitan monostearate) were purchased from Millipore Sigma. TWEEN® 80 (polyethylene glycol sorbitan monooleate) and sodium citrate dihydrate were purchased from Fisher Chemical. The chloride salt of the cationic lipid 1,2-dioleoyl-3-trimethylammonium-propane (DOTAP chloride) was purchased from Corden Pharma. Ultrapure water (18.2 MOhm-cm resistivity) was obtained from a Milli-Q water purification system (Millipore Sigma).

Lipid carriers were prepared which comprised 37.5 mg/ml squalene, 37 mg/ml SPAN® 60, 37 mg/ml TWEEN® 80, 30 mg/ml DOTAP chloride, 0.1 mg/ml 10 nm iron oxide nanoparticles and 10 mM sodium citrate dihydrate.

The lipid carriers were manufactured using the following procedures. In a 200 ml beaker, 0.4 ml of iron oxide nanoparticles at 25 mg Fe/ml in chloroform, with a number-weighted average diameter of 10 nm, were added.

Chloroform was allowed to evaporate in a fume hood leaving behind a dry coating of iron oxide nanoparticles. To the iron oxide nanoparticles, 3.7 grams of SPAN® 60, 3.75 grams of squalene, and 3 grams of DOTAP chloride were added to prepare the “oil” phase.

The oil phase was sonicated 30 minutes in a water bath pre-heated to 60° C. Separately, in a 1 liter glass bottle, the “aqueous” phase was prepared by adding 39 grams of TWEEN® 80 to 1,000 ml 10 mM sodium citrate dihydrate solution prepared with Milli-Q water.

The aqueous phase was stirred for 30 minutes to allow complete dissolution of TWEEN® 80. After complete dissolution of TWEEN® 80, 96 ml of the aqueous phase was transferred to a 200 ml beaker and incubated in a water bath pre-heated to 60° C. To the heated oil phase, 96 ml of the pre-heated aqueous phase was added. The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi. The fluid was passaged until the z-average hydrodynamic diameter, measured by dynamic light scattering (Malvern Zetasizer Nano S), was 54 nm with a 0.2 polydispersity index. The microfluidized lipid carrier sample was terminally filtered with a 200 nm pore-size polyethersulfone (PES) syringe filter.

### Example 3: Techniques and Materials for Producing Lipid Carriers with 15 Nm Iron Oxide NPS

Lipid carriers were prepared which comprised 37.5 mg/ml squalene, 37 mg/ml SPAN® 60, 37 mg/ml TWEEN® 80, 30 mg/ml DOTAP chloride, 0.2 mg/ml 15 nm iron oxide nanoparticles, and 10 M sodium citrate dihydrate. Lipid carriers were manufactured using the following procedures.

In a 200 ml beaker, 0.8 ml of iron oxide nanoparticles at 25 mg Fe/ml in chloroform, with a number-weighted average diameter of 15 nm, was added. Chloroform was allowed to evaporate in a fume hood leaving behind a dry coating of iron oxide nanoparticles. To the iron oxide nanoparticles, 3.7 grams of SPAN® 60, 3.75 grams of squalene, and 3 grams of DOTAP chloride were added to prepare the “oil” phase.

The oil phase was sonicated 30 minutes in a water bath pre-heated to 60° C. Separately, in a 1 liter glass bottle, the “aqueous” phase was prepared by adding 39 grams of TWEEN® 80 to 1,000 ml of 10 mM sodium citrate dihydrate solution prepared with Milli-Q water. The aqueous phase was stirred for 30 minutes to allow complete dissolution of TWEEN® 80.

After complete dissolution of TWEEN® 80, 96 ml of the aqueous phase was transferred to a 200 ml beaker and incubated in a water bath pre-heated to 60° C. To the heated oil phase, 96 ml of the pre-heated aqueous phase was added. The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi.

The fluid was passaged until the z-average hydrodynamic diameter, measured by dynamic light scattering (Malvern Zetasizer Nano S), was 52 nm with a 0.2 polydispersity index. The microfluidized lipid carrier sample was terminally filtered with a 200 nm pore-size polyethersulfone (PES) syringe filter.

### Example 4: Immune Response in Macrophages

Various formulations of lipid carrier and repRNA were prepared and analyzed to assay innate immune response of the lipid carrier in macrophages. Protein expression and stimulation of TNF production in THP-1 macrophages was studied.

Initially, the THP-1 monocytes were differentiated into macrophages using phorbol 12-myristate 13-acetate (PMA). The cells were then transfected with various formulations with Nano Luciferase encoding replicon RNA (SEQ ID NO: 28). The cell culture media was then assessed for NanoLuc and TNF expression.

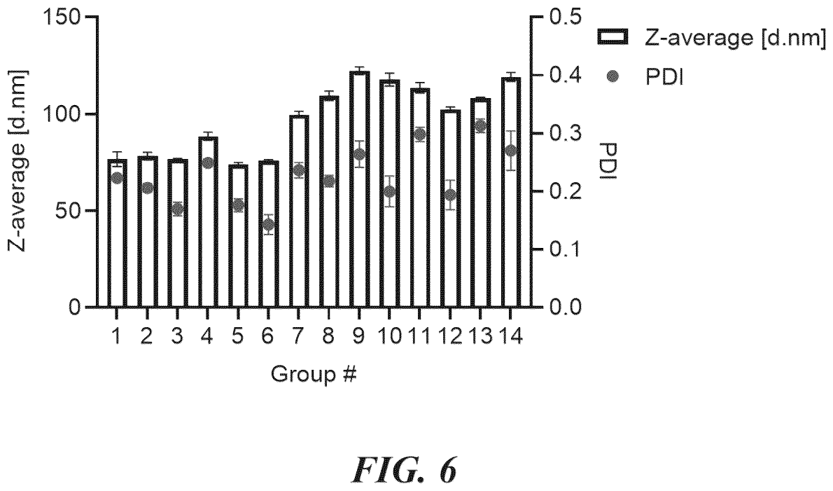

The formulations and their characteristics such as particle size and PDI that were used in this assay are described in Table 5 and FIG. 6. The concentration of repRNA encoding NanoLuc was 909 ng/μl and maintained at −80 degrees Celsius. MIGLYOL® 812 N, a triglyceride ester of saturated coconut/palm-kernel oil derived caprylic and capric fatty acids and plant derived glycerol was used in this assay.

TABLE 5
Formulations.

| | Particle | | | | | | | |
|---|---|---|---|---|---|---|---|---|
| | size | | Iron | Aluminum | | | | |
| | [diameter, | | [mg | [mg | DOTAP | Squalene | MIGLYOL | Solanesol |
| Formulation | nm] | PDI | Fe/ml] | Al/ml] | [mg/ml] | [mg/ml] | [mg/ml] | [mg/ml] |
| Fe-lipid carrier | 59.3 | 0.23 | 0.19 | n/a | 27.9 | 39.4 | n/a | n/a |
| High Fe-lipid | 57.5 | 0.24 | 0.85 | n/a | 29.1 | 40.5 | n/a | n/a |
| carrier | | | | | | | | |
| Fe-lipid carrier | 48.7 | 0.2 | 0.18 | n/a | 28.3 | n/a | not | n/a |
| MIGLYOL | | | | | | | measured | |
| High Fe-lipid | 62.6 | 0.28 | 0.94 | n/a | 27.7 | n/a | not | n/a |
| carrier | | | | | | | measured | |
| MIGLYOL | | | | | | | | |
| Alum-lipid | 64.5 | 0.25 | n/a | 0.88 | 27.4 | 41.2 | n/a | n/a |
| carrier | | | | | | | | |
| Fe-lipid carrier | 86.1 | 0.26 | 0.16 | n/a | 26.2 | n/a | n/a | 36 |
| solanesol (SLN) | | | | | | | | |
| NLC | 50 | 0.26 | n/a | n/a | 26.7 | 34.1 | n/a | n/a |
| CNE | 105.4 | 0.06 | n/a | n/a | 4.4 | 47.4 | n/a | n/a |
| lipid carrier | 54.2 | 0.22 | n/a | n/a | 19.3 | 32.6 | n/a | n/a |
| (w/o IO) | | | | | | | | |

### Example 5: Fe-Lipid Carrier Formulation-NP-1 (Prepared at 100 ml Scale)

Fe-lipid carrier formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 0.2 mg Fe/ml 12 nm oleic acid-coated iron oxide nanoparticles (Imagion Biosystems, San Diego, CA, USA) and 10 mM sodium citrate dihydrate (Fisher Chemical). 1 ml of 20 mg Fe/ml 12 nm diameter oleic acid-coated iron oxide nanoparticles in chloroform (Imagion Biosystems, lot #95-127) were washed three times by magnetically separating in a 4:1 acetone: chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree C. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International, Radnor, PA, USA) and the resulting crude emulsion was processed by passaging through a M110P microfluidizer (Microfluidics, Westwood, MA, USA) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.25 polydispersity index (PDI). The microfluidized NP-1 formulation was terminally filtered with a 200 nm pore-size polyethersulfone (PES) filter and stored at 2 to 8 degrees C. Iron concentration was determined by inductively coupled plasma-optical emission spectrometry (ICP-OES). DOTAP and squalene concentration were measured by reverse phase high-performance liquid chromatography (RP-HPLC).

### Example 6: High Fe-Lipid Carrier Formulation Np-2 (Prepared at 100 ml Scale)

High Fe-lipid carrier formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 1 mg Fe/ml 15 nm oleic acid-coated iron oxide nanoparticles (Imagion Biosystems) and 10 mM sodium citrate dihydrate (Fisher Chemical). 5 ml of 20 mg Fe/ml 15 nm diameter oleic acid-coated iron oxide nanoparticles in chloroform (Imagion Biosystems, Lot #95-133) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree Celsius water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP and Squalene concentration were measured by RP-HPLC.

### Example 7: Fe-Lipid Carrier Miglyol Formulation NP-3 (Prepared at 100 ml Scale)

The Fe-lipid carrier miglyol formulation comprises 37.5 mg/ml Miglyol 812 N (IOI Oleo GmbH), 37 mg/ml SPAN® 60 (Millipore Sigma), 37 mg/ml TWEEN® 80 (Fisher Chemical), 30 mg/ml DOTAP chloride (LIPOID), 0.2 mg Fe/ml 15 nm oleic acid-coated iron oxide nanoparticles (Imagion Biosystems) and 10 mM sodium citrate dihydrate (Fisher Chemical). 1 ml of 20 mg Fe/ml 15 nm diameter oleic acid-coated iron oxide nanoparticles in chloroform (Imagion Biosystems, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree C. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degree C. for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP concentration was measured by RP-HPLC.

### Example 8: High Fe-Lipid Carrier Miglyol Formulation NP-4 (Prepared at 100 ml Scale)

High Fe-lipid carrier Miglyol formulation comprises 37.5 mg/ml Miglyol 812 N (IOI Oleo GmbH), 37 mg/ml SPAN® 60 (Millipore Sigma), 37 mg/ml TWEEN® 80 (Fisher Chemical), 30 mg/ml DOTAP chloride (LIPOID), 1 mg/ml 15 nm oleic acid-coated iron oxide nanoparticles (ImagionBio) and 10 mM sodium citrate dihydrate (Fisher Chemical). 5 ml of 20 mg Fe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN® 60, and 3 grams DOTAP were added to produce the oil phase. The oil phase was sonicated for 45 minutes in a 65 degree C. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP concentration was measured by RP-HPLC.

### Example 9: Alum-Lipid Carrier Formulation NP-5 (Prepared at 100 ml Scale)

Alum-lipid carrier formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 1 mg Al/ml TOPO-coated Alhydrogel® (aluminum oxyhydroxide) particles (Croda) and 10 mM sodium citrate. 10 ml of Alhydrogel was washed three times in methanol by centrifuging at 1000 rpm for 20 minutes. After the third wash, Alhydrogel was dispersed in 10 ml methanol and to this dispersion was added 1 ml of 250 mg/ml trioctylphosphine oxide (TOPO) and incubated overnight in a 37° C. orbital shaker. Excess TOPO was removed by additional methanol washes and then dispersed in 11 ml methanol. Methanol was allowed to evaporate overnight in the fume hood leaving behind a dry layer of TOPO-Alhydrogel. To this dry TOPO-Alhydrogel layer, 3.75 grams squalene, 3.7 grams SPAN® 60, and 3 grams DOTAP were added to produce the oil phase. The oil phase was sonicated for 45 minutes in a 65 degree C. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Aluminum concentration was determined by ICP-OES. DOTAP and Squalene concentration were measured by RP-HPLC.

### Example 10: Fe-Lipid Carrier Solanesol Formulation NP-6 (Prepared at 100 ml Scale)

Fe-lipid carrier solanesol formulation (NP-6) comprises 37.5 mg/ml Solanesol (Cayman chemicals), 37 mg/ml SPAN® 60 (Millipore Sigma), 37 mg/ml TWEEN® 80 (Fisher Chemical), 30 mg/ml DOTAP chloride (LIPOID), 0.2 mg Fe/ml oleic acid-coated iron oxide nanoparticles (ImagionBio) and 10 mM sodium citrate. 1 ml of 20 mg Fe/ml 15 nm diameter oleic acid-coated iron oxide nanoparticles in chloroform (ImagionBio, Lot #95-133) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams solanesol, 3.7 grams SPAN® 60, and 3 grams DOTAP were added to produce the oil phase. The oil phase was sonicated for 45 minutes in a 65 degree C. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber. The microfluidized formulation was terminally filtered with a 200 nm pore-size polyethersulfone (PES) filter and stored at 2 to 8 degrees C. Iron concentration was determined by ICP-OES. DOTAP and solanesol concentration were measured by RP-HPLC.

### Example 11: NP-7 Formulation (Prepared at 100 ml Scale)

NP-7 formulation comprises 37.5 mg/ml squalene (SEPPIC), 37 mg/ml SPAN® 60 (Millipore Sigma), 37 mg/ml TWEEN® 80 (Fisher Chemical), 30 mg/ml DOTAP chloride (LIPOID), 2.4 mg/ml Dynasan 114 (101 Oleo GmbH) and 10 mM sodium citrate. To a 200 ml beaker 3.75 grams squalene, 3.7 grams SPAN® 60, 0.24 grams Dynasan 114 and 3 grams DOTAP were added to produce the oil phase. The oil phase was sonicated for 45 minutes in a 65 degree C. water bath. Separately, the aqueous phase was prepared by dissolving 19.5 grams TWEEN® 80 in 500 ml of 10 mM sodium citrate buffer prepared in nuclease free water. 92 ml of the aqueous phase was transferred to a separate glass bottle and heated to 65 degrees Celsius for 30 minutes. The oil phase was mixed with the 92 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. DOTAP and squalene concentration were measured by RP-HPLC.

### Example 12: NP-8 Formulation (Prepared at 100 ml Scale)

The NP-8 formulation comprises 43 mg/ml squalene (SEPPIC), 5 mg/ml SPAN® 85 (Millipore Sigma), 5 mg/ml TWEEN® 80 (Fisher Chemical), 4 mg/ml DOTAP chloride (LIPOID) and 10 mM sodium citrate. To a 200 ml beaker 4.3 grams squalene, 0.5 grams SPAN® 85, and 0.4 grams DOTAP were added to produce the oil phase. The oil phase was sonicated for 45 minutes in a 65 degree C. water bath. Separately, the aqueous phase was prepared by dissolving 2.6 grams TWEEN® 80 in 500 ml of 10 mM sodium citrate buffer prepared in nuclease free water. 95 ml of the aqueous phase was transferred to a separate glass bottle and heated to 65 degrees Celsius for 30 minutes. The oil phase was mixed with the 95 ml of aqueous phase by adding the warm oil phase to the warm aqueous phase. The mixture was emulsified using a VWR® 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 100±10 nm with a 0.05-0.1 polydispersity index (PDI). The microfluidized formulation was terminally filtered with a 200 nm pore-size polyethersulfone (PES) filter and stored at 2 to 8 degrees C. DOTAP and Squalene concentration were measured by RP-HPLC.

The treatment groups were prepared. Eight of those groups were NanoLuc repRNA groups, with 600 ng dose per well was prepared using the Fe-lipid carrier, High Fe-lipid carrier, Fe-lipid carrier miglyol, High Fe-lipid carrier miglyol, Alum-lipid carrier, Fe-lipid carrier solanesol (SLN), NLC, and CNE formulations. The untreated group did not have NanoLuc. The various formulations were prepared by diluting NanoLuc repRNA to 8 ng/μL in 2.2 mL of RNAse-free water. The lipid carrier formulations and RNA master mix were complexed by adding 250 μL of each diluted formulation with 250 μL of diluted RNA, and mixed by pipetting up and down.

Cell transfections were carried out by seeding 7×10⁵ THP-1s per well in a 24-well plate. 80 micromolar (μM) PMA added to each well and incubated at 37 degrees Celsius. The next day, the PMA-containing media was removed and replaced with complete RPMI (cRPMI) medium for one hour before transfection. The samples were then serially diluted in Opti-MEM™ (Thermo Fisher Scientific, Waltham, MA USA) to make a 10-point 1.5-fold dilution series starting at 0.45 ng/μL. The culture media was then removed from the plates by pipetting. 450 μL of Opti-MEM™ and 150 μL of the complexed formulation were added to the plate in duplicate. The empty wells were given 450 μL of Opti-MEM™ only. After four hours, the samples were removed from the plate by pipetting and replaced with 500 μL of growth media. The plate was then incubated overnight at 37 degrees Celsius. The growth media was harvested the next day and stored at −80 degrees Celsius. Downstream assays were conducted and described below.

The luciferase assay was performed by first diluting the Nano-Glo® luciferase assay reagent 1:50 in buffer. 25 μL of supernatant was removed and mixed with 25 μL of Nano-Glo® reagent in a 96-well plate. This was incubated at room temperature for 3 minutes. The luminescence was read using a luminometer.

Next, an ELISA was performed to evaluate the TNF-alpha (a) protein levels in cell culture media using the human TNF-α DUOSET™ ELISA (R&D Systems) according to the manufacturer's protocol. The 96-well microplate was coated with anti-TNFα capture antibody. The plate was blocked and then media samples were added directly without dilution. After addition of the biotinylated detection antibody, SA-HRP, and substrate, the absorbance was read at 450 nm on a SPECTRAMAX® i3 (Molecular Devices) plate reader.

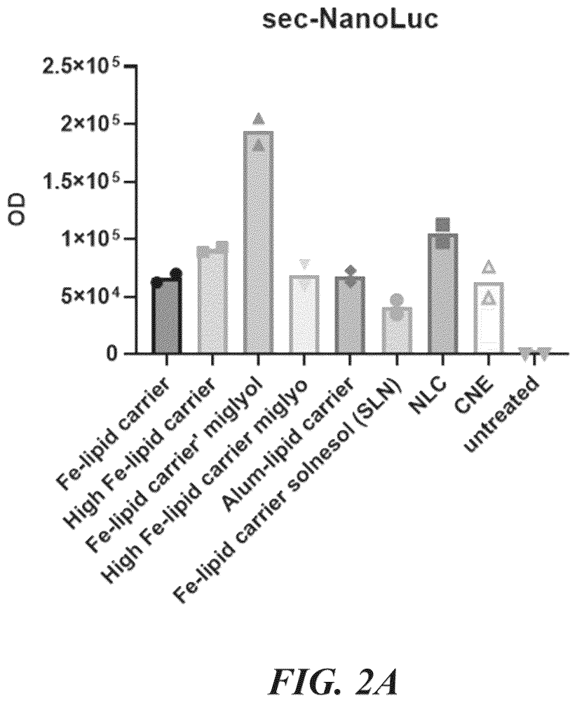

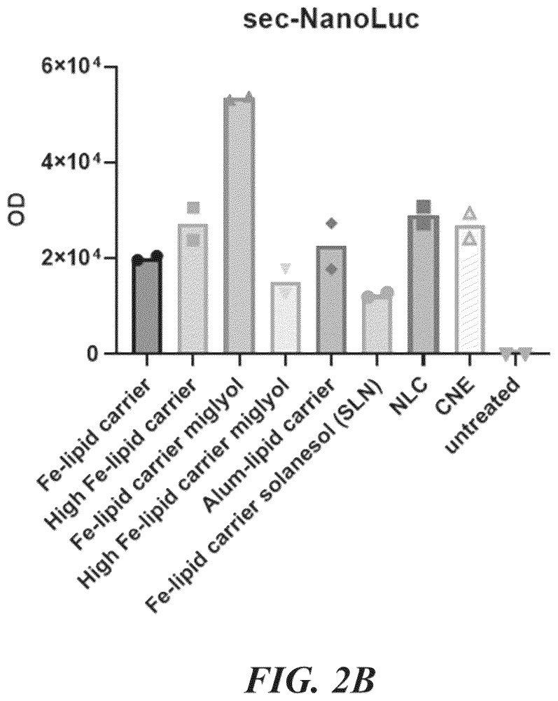

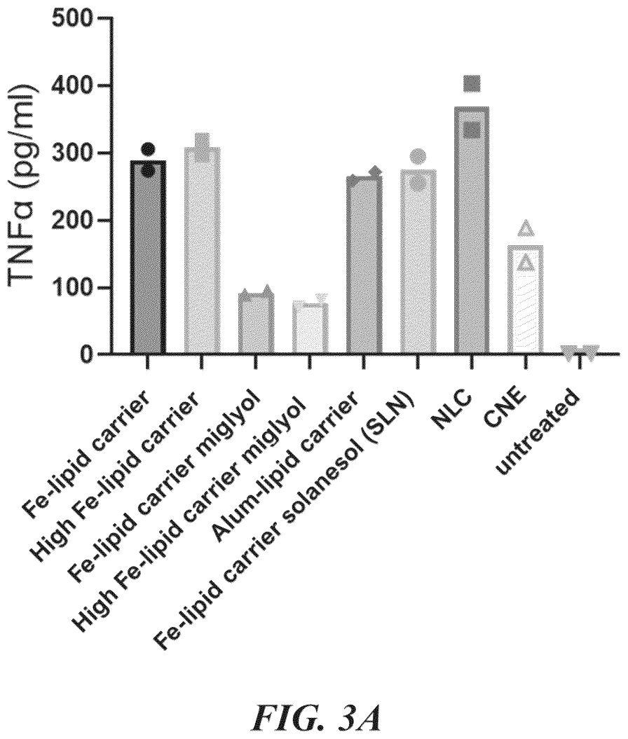

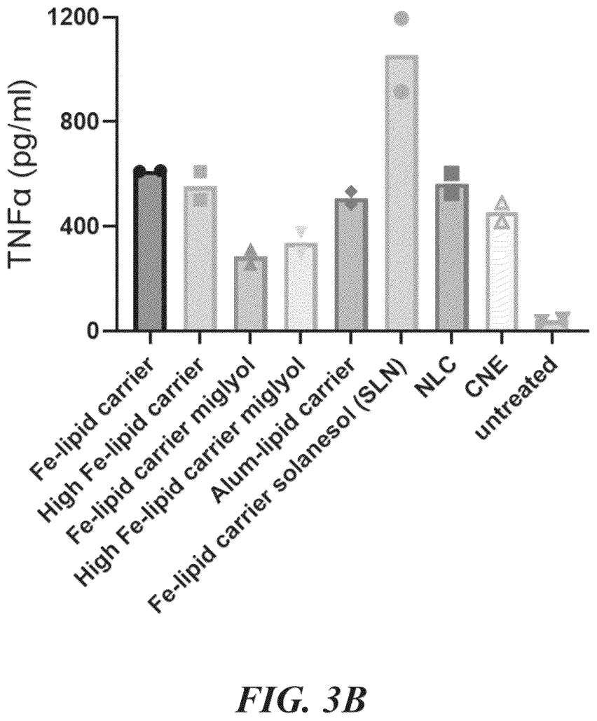

All studies in this example were done in duplicates. Results from the duplicates are presented as first assay and second assay, respectively. The formulation comprising a lipid carrier and miglyol induced higher protein production off the replicon, as shown in the first assay in FIG. 2A and in the second assay in FIG. 2B. A reduced innate immune response was detected, as measured by TNF-α secretion (FIG. 3A-3B).

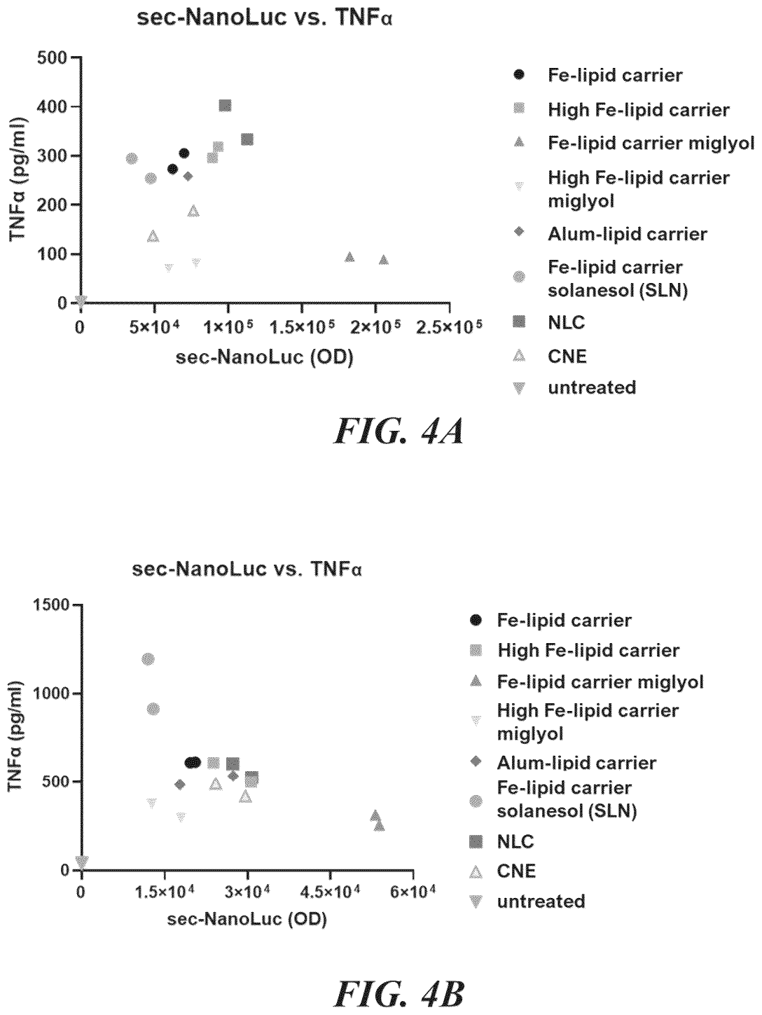

The correlation between enhanced protein production and low TNF-alpha stimulation was observed with the miglyol lipid carrier formulation, as shown in the first assay in FIG. 4A and in the second assay in FIG. 4B. The solanesol lipid carrier formulation induced slightly lower protein production, but a higher TNFα production (FIGS. 4A-4B). Correlation data from the first assay is shown in FIG. 4A are derived from the data represented in FIG. 2A and FIG. 3A. Correlation data from second assay is shown in FIG. 4B, which is derived from the data represented in FIG. 2B and FIG. 3B.

### Example 13: Techniques and Materials for Producing Lipid Carriers with DNA Encoding Sear

C57BL/6 mice were inoculated as described in Table 6 below. After which secreted embryonic alkaline phosphatase (SEAP) levels were measured in serum. A summary of the materials used in the example is provided in Table 6.

TABLE 6
Materials.

| Description | Concentration | | Temperature/Location |
|---|---|---|---|
| DNA encoding SEAP | 5 | mg/mL | −20° C. |
| (DNA sequence as set | | | |
| forth in SEQ ID NO: 33) | | | |
| repRNA encoding SEAP | 2217 | μg/mL | −80° C. |
| Lipid carrier formulation | 30 | mg | 4° C. |

DOTAP/mL

TABLE 7
Treatment Groups.

| | | | | RNA | DNA | | Injection |
|---|---|---|---|---|---|---|---|
| | | | DNA/RNA- | dose | dose | | Volume |
| Group | n | Formulation | SEAP | [μg] | [μg] | N:P | [μL] |
| 1 | 5 | Naked | DNA-SEAP | | 20 | n/a | 50 |
| 2 | 5 | Lipid carrier | DNA-SEAP | | 10 | 15 | 50 |
| 3 | 5 | Lipid carrier | DNA-SEAP | | 10 | 7.5 | |
| 4 | 5 | Lipid carrier | DNA-SEAP | | 20 | 15 | |
| 5 | 5 | Lipid carrier | DNA-SEAP | | 20 | 7.5 | |
| 6 | 5 | Lipid carrier | RNA-SEAP | 1 | | 15 | 50 |
| 7 | 5 | Miglyol + lipid | RNA-SEAP | 1 | | 15 | 50 |
| | | carrier | | | | | |

Seven different formulations were prepared and administered intramuscularly across the seven treatment groups (Groups 1-7) as shown in Table 7. DNA-SEAP or RNA-SEAP was diluted according to the volumes set forth in Table 8 to prepare the formulations for Groups 1-7.

TABLE 8
Formulation Preparation; Dilution of RNA/DNA

| | | DNA or | 40% | | |
|---|---|---|---|---|---|
| | DNA- or | RNA | sucrose | water | Total |
| Group | RNA-SEAP | [μL] | [μL] | [μL] | [μL] |
| 1 | DNA-SEAP | 40.0 | 125.0 | 85.0 | 250.0 |
| 2 | DNA-SEAP | 20.0 | 0.0 | 230.0 | 250.0 |
| 3 | DNA-SEAP | 20.0 | 0.0 | 230.0 | 250.0 |
| 4 | DNA-SEAP | 40.0 | 125.0 | 85.0 | 250.0 |
| 5 | DNA-SEAP | 40.0 | 0.0 | 210.0 | 250.0 |
| 6 | RNA-SEAP | 4.5 | 0.0 | 245.5 | 250.0 |
| 7 | RNA-SEAP | 4.5 | 0.0 | 245.5 | 250.0 |

The concentrations of diluted DNA or RNA prior to complexing with the lipid carrier was as follows (measured by NanoDrop spec): Groups 1, 4 and 5 contains about 820 μg/ml DNA; Groups 2 and 3 contained about 480 μg/ml DNA; and Groups 6 and 7 contained about 43 μg/ml RNA.

Formulations for Groups 1-7 were diluted with 100 mM citrate as set forth in Table 9 below.

TABLE 9
Dilution of lipid carrier formulations.

| | | Lipid | 40% | 100 mM | | |
|---|---|---|---|---|---|---|
| | | Carrier | sucrose | citrate | Water | Total |
| Group | Formulation | [μl] | [μl] | [μl] | [μl] | [μl] |
| 1 | Naked | 0 | 0 | 30 | 270 | 300 |
| 2 | Lipid carrier | 120 | 150 | 30 | 0 | 300 |
| 3 | Lipid carrier | 60 | 150 | 30 | 60 | 300 |
| 4 | Lipid carrier | 240 | 0 | 30 | 30 | 300 |
| 5 | Lipid carrier | 120 | 150 | 30 | 0 | 300 |
| 6 | Lipid carrier | 12 | 150 | 30 | 108 | 300 |
| 7 | Miglyol + | 12 | 150 | 30 | 108 | 300 |
| | lipid carrier | | | | | |

The above formulations were complexed by adding 250 μl diluted lipid carrier to 250 μl diluted DNA or RNA. The resulting complexed formulations were incubated on ice for at least 30 minutes. Table 10 sets forth the experiment schedule for the assay.

TABLE 10
Experiment schedule.

| Day | Procedure | Notes on Mice |
|---|---|---|
| 0 | All inoculations | None |
| 4 | Bleed | Group 4 had ruffled fur, one mouse |
| | | emaciated (died during collection). |
| | | Hydropaque placed in cage. |
| 6 | Bleed | None |
| 8 | Bleed | None |
| 11 | Bleed | None |
| 14 | Bleed | None |

Mice were bled at regular intervals and serum was prepared immediately and stored at −80 degrees Celsius until analyses for SEAP activity.

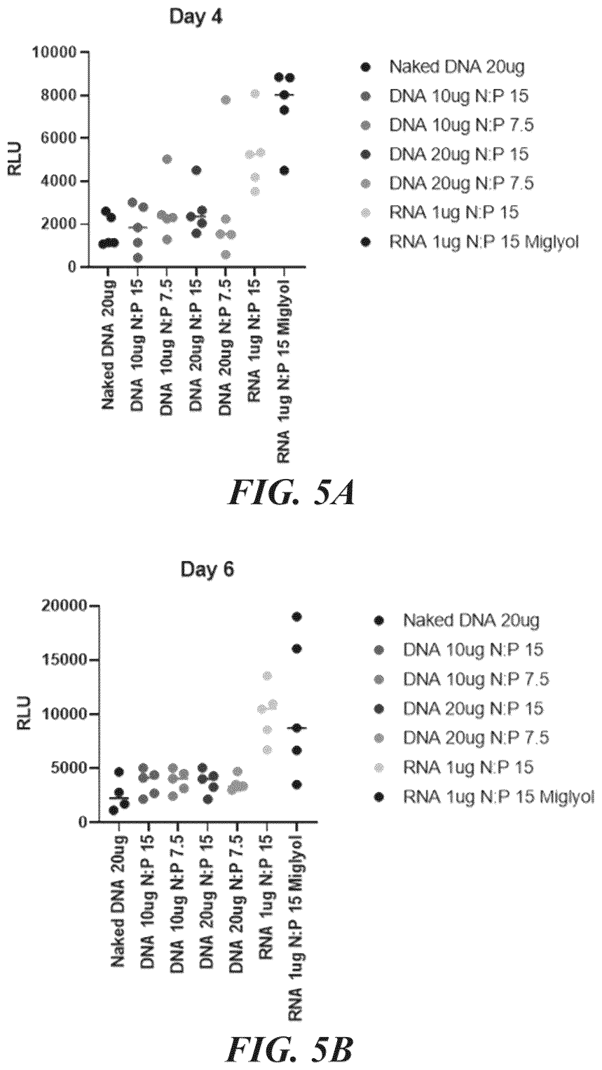

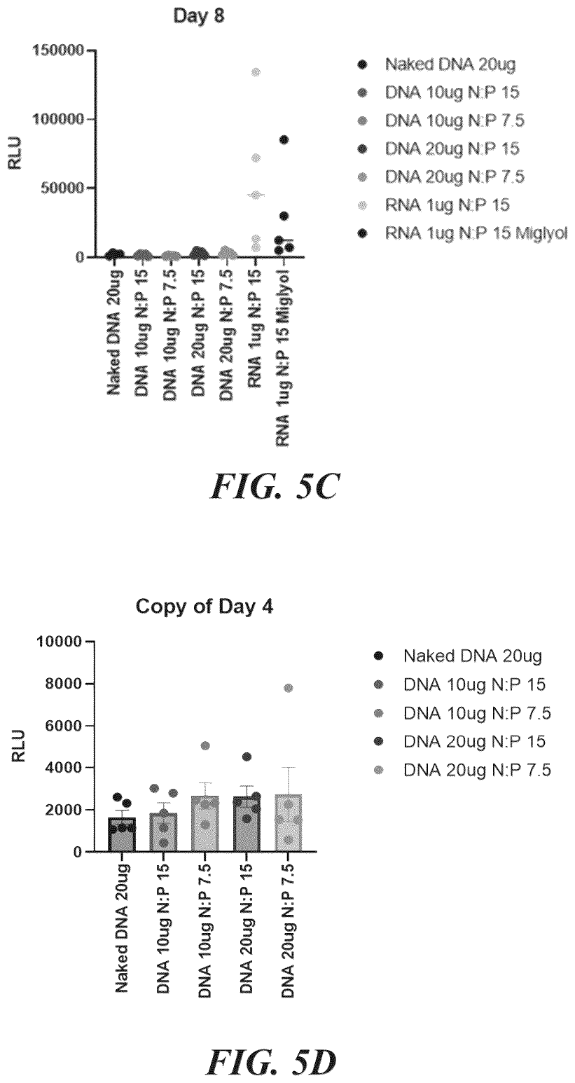

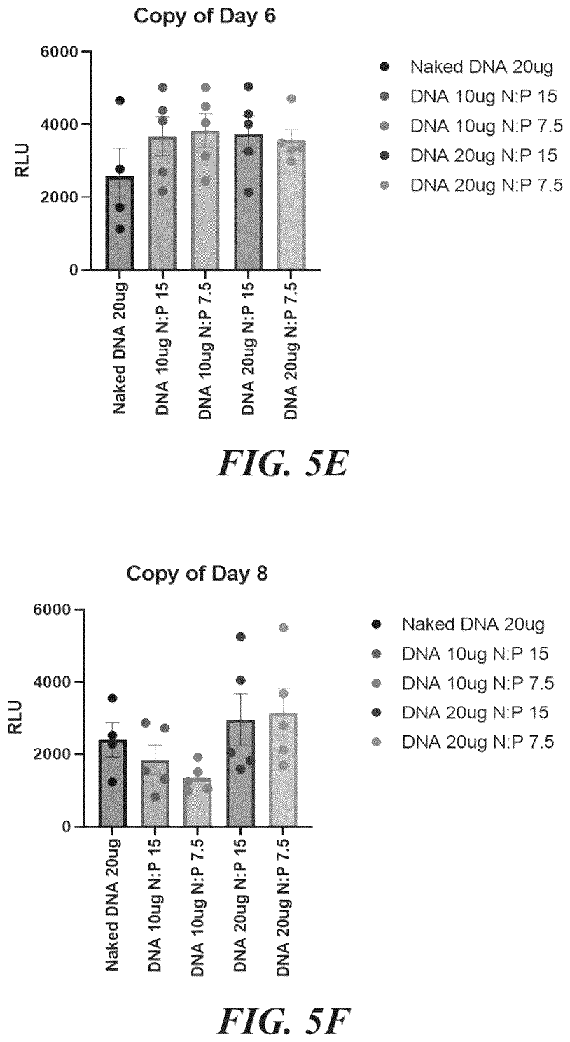

To evaluate SEAP levels in serum, all serum samples were thawed at the same time and SEAP detection was conducted. FIGS. 5A-5F illustrate the SEAP levels in BALB/c mice injected intramuscularly with varying iterations of lipid carrier-formulated DNA SEAP. Mice were bled at regular intervals, serum prepared and stored until analysis by SEAP assay. Data are displayed as mean and SE (n=5 per group).

As can be seen from FIGS. 5A-5F, lipid carrier formulations aid target protein production over delivery of DNA alone, particularly after day 6 following injection. Additionally, the data shows that inclusion of miglyol enhances protein production from an RNA replicon over lipid carrier formulations lacking miglyol.

### Example 14: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 28.

### Example 15: Additional Nanoparticle Formulations

Additional nanoparticle formulations are produced according to the following tables (Table 11 and Table 12).

TABLE 11
mRNA - Nanoparticle Formulation.

| | Dosage form: | | | |
|---|---|---|---|---|
| | Solution for Injection for IM route of administration | | | |
| | Each 0.5 ml | | | Concentration |
| Composition: | Vial Contains: | Quantity | | (mg/ml) |
| | mRNA | 25 | mcg | 0.05 |
| | DOTAP | 0.75 | mg | 1.5 |
| | Iron Oxide Nanoparticles | 0.005 | mg | 0.01 |
| | Squalene | 0.94 | mg | 1.88 |
| | Sorbitan Monostearate | 0.93 | mg | 1.86 |
| | Polysorbate 80 | 0.93 | mg | 1.86 |
| | Sucrose IP | 50 | mg | 100 |
| | Citric Acid Monohydrate | 1.05 | mg | 2.1 |
| | Water for Injection | q.s. to 0.5 | ml | |

TABLE 12
Lyophilized mRNA Formulation.

| | Dosage form: | | | | |
|---|---|---|---|---|---|
| | Lyophilized powder | | | | |
| Com- | Each 5 dose vial | | | Concentration | Approximate |
| position: | contains: | Quantity | | (mg/ml) | dry weight % |
| | mRNA | 50 | mcg | 0.02 | 0.02 |
| | DOTAP | 1.5 | mg | 0.6 | 0.57 |
| | Squalene | 1.88 | mg | 0.752 | 0.72 |
| | Sorbitan | 1.86 | mg | 0.744 | 0.71 |
| | Monostearate | | | | |
| | Polysorbate 80 | 1.86 | mg | 0.744 | 0.71 |
| | Sucrose IP | 250 | mg | 100 | 95.3 |
| | Citric Acid | 5.25 | mg | 2.1 | 2 |
| | Monohydrate | | | | |
| | Water for Injection | 2.5 ml | | | |
| | (for reconstitution) | | | | |

### Example 16: Tumor Therapy by Co-Delivery of a IL-12 Nucleic Acid and Immunomodulators

Immune system modulators were developed for co-delivery with IL-12 as follows. IL-12 for co-delivery with a TLR3 agonist, a RIG-I agonist (PAMP/RAR), and a TLR4 agonist were generated by the methods provided below. The RNA was either co-formulated, co-delivered, or separated in time from the immune system modulator.

Self-replicating mRNA generation: Plasmids encoding self-replicating mRNA (srRNA) based on a modified Venezuelan Equine Encephalitis alphavirus (VEE, TC-83 strain) were constructed by inserting the gene for murine IL-12 downstream of the VEE subgenomic promoter in place of the VEE structural polyprotein open reading frame (SEQ ID NO: 32, FIG. 7A). Self-replicating mRNA was produced using standard in vitro. Run-off mRNA transcription (MegaScript T7, ThermoFisher, Waltham, MA) and post-transcription capping protocols (Cap1 Kit, CellScript, Madison, WI), following by LiCl purification.

TLR4 agonist preparation: The TLR4 agonist, glucopyranosyl lipid A (GLA), was formulated in a stable, 10% squalene oil-in water emulsion. GLA-SE refers to formulation that has been diluted to 2% stable oil-in-water emulsion with Hanks' Balanced Salt Solution (HBSS).

Complex formation: srRNA was formulated with the transfection reagent, In Vivo jetPEI® (Polyplus, Strasbourg, France), according to the manufacturer's instructions. Briefly, srRNA was mixed gently with jetPEI® in a 5% glucose diluent and complexed by brief incubation at room temperature. The addition of GLA-SE, followed RNA complex formation.

Animal Models and Assays: Female 7-week-old C57BL/6 or BALB/c mice were housed in a BSL2+ level room under reduced-pathogen conditions. Mice were anesthetized and inoculated with syngeneic cancer cell lines. For the B16 melanoma model, C57BL/6 mice were inoculated subcutaneously either in the flank or footpad with 1×10⁵ or 1×10⁶ B16-F10 murine melanoma cells, respectively. Intratumoral injections were performed once tumors were palpable, usually 7-10 days following implantation. Prior to intratumoral injection, LVs and formulated RNA were diluted to a total volume of 50 μL/injection in cold HBSS or 5% glucose, respectively, and kept on ice. srRNA/mIL12 were administered once weekly, while LVs were administered. When GLA-SE was included in the treatment regimen, it was diluted to a 2% stable emulsion prior to the injection and administered biweekly starting 24 hours after the single injection of LV/mIL12 or concomitantly with formulated srRNA/mIL12.

In-life monitoring of serum content of IL-12 or IFNγ was conducted by retro-orbital collection of whole blood into serum collection tubes (BD Biosciences, Waltham, MA), not exceeding 10% of the mouse total blood volume. Whole blood was then centrifuged to remove cells and serum was collected and stored at −20° C. until analysis. ELISAs for detection of IL-12 and IFNγ were used for quantification (ThermoFisher 88-7121-22 and 88-7314-22, LLOQ=4 μg/ml).

Following flank inoculation of B16 melanoma tumors, mice were either treated or left untreated (control mice). Around 18 days post-inoculation, mice were euthanized and tumors were isolated using scissors and forceps and dissociated using a GentleMACS (Miltenyi, Bergisch Gladbach, Germany). Tumor cell suspensions were then added to a Ficoll gradient using a SepMate tube system (StemCell Technologies, Vancouver, CA) and centrifuged for 10 minutes at 1200×g. Tumor cells were pelleted, while infiltrating immune populations were isolated in the top layer of the gradient. These cells were washed to remove Ficoll, then stained using a combination of FITC-Ly6G, FITC-NKp46, PE-CD11b, PE-Foxp3, PerCP-Cy5.5 F480, PerCP-Cy5.5 CD3e, APC-Ly6C, APC-CD25, AlexaFluor700-CD4, eFluor-450-CD8, PacBlueCD11c, BV500-B220 or BV500-CD8a and Live Dead near-IR stain (Biolegend, San Diego, CA or ThermoFisher, Waltham, MA).

Around 18 days post-inoculation, treated or untreated B16 melanoma tumors were removed from sacrificed mice using scissors and forceps and dissociated using the RNA isolation setting of a GentleMACS (Miltenyi, Bergisch Gladbach, Germany). RNA was isolated from tumor lysates using a RNeasy Mini Kit (Qiagen, Hilden, Germany) and NanoString™ (NanoString Technologies, Inc., Seattle WA, analysis using the murine PanCancer Immune Oncology panel of 770 genes was performed. Data analysis was performed using the Advanced Analysis tool of nSolver. Gene expression changes were scored in comparison to the mean expression across the entire assay. From there, gene scores were batched by function or cell phenotype. Gene score batches were then clustered hierarchically based on the degree to which the genes changed.

For in vivo imaging of tumors, mice were injected with 150 mg/kg D-luciferin (Perkin Elmer, Waltham, MA) dissolved in HBSS, intraperitoneally. Mice were then imaged using an IVIS® optical imaging system 15-20 minutes following injection.

### Example 17: Cytokine and Innate Immune Response Modulators Complexed with Lipid Nanoparticles

The following describes delivery of a cytokine and an innate immune response modulator by a lipid carrier. Self-replicating RNA encoding for IL-12 and a TLR4 agonist are complexed with NP-1, NP-30, and NP-31. The RNA encoding IL-12 is complexed separately with the TLR4 agonist. Briefly, complexes of an RNA and the TLR4 agonist with nanoparticle NP-1 are generated, having nitrogen-to-phosphate (N:P) molar ratio of 25, 5, 1 or 0.2. The N:P ratio is the ratio of positively charged nitrogens (N) on NP-1 formulation to negatively charged phosphates on the RNA (P). The RNA concentration is measured by nanodrop. N is determined by the amount of cationic lipid in the nanoparticle, and DOTAP in the case of NP-1. The complexed reagents are incubated on ice for 30 minutes. Half the complex is run on an RNA gel electrophoresis to assess unbound RNA, and visually assessed following an image capture. Assessment of the image is to confirm no naked nucleic acid present when the RNA and nanoparticle are mixed, indicating the RNA is able to complex with the nanoparticle.

To the remaining complex sample, RNase challenge is performed, followed by treatment with proteinase to quench the reaction. The RNA is extracted from the surviving complex using a phenol chloroform extraction. The aqueous phase is mixed with glyoxal running buffer and run on an RNA gel electrophoresis and visually assessed following an image capture. Assessment is to show the RNA molecule is protected from RNase activity.]

### Example 18: IL-12 Replicon RNA Efficacy in a Syngeneic Mouse Model for Melanoma

### Nanoparticle Formulations and Methods of Generating NP-30, NP-1, and LNP.

NP-30 lipid carriers were prepared. Briefly, the oil phase (squalene, Span 60, and DOTAP) was sonicated for 30 min in a 65° C. water bath. Separately, the aqueous phase, containing Tween 80 and sodium citrate dihydrate solution in water, was prepared with continuous stirring until all components were dissolved. The oil and aqueous phases were then mixed and emulsified using a VWR 200 homogenizer (VWR International), and the crude colloid was subsequently processed by passaging through a microfluidizer at 137895 kPa with an LM10 microfluidizer equipped with an H10Z 100-μm ceramic interaction chamber (Microfluidics) until the Z-average hydrodynamic diameter, measured by dynamic light scattering (Malvern Zetasizer Nano S), reached 50±5 nm with a 0.2 polydispersity index. The microfluidized NP-30 was terminally filtered with a 200-nm pore-size polyethersulfone filter and stored at 2° to 8° C.

NP-1 was prepared according to Example 5 to include iron within the hydrophobic core.

A lipid nanoparticle (LNP) was prepared. Briefly, lipid components were dissolved in ethanol at a ratio of 50:10:38:2 (Ionizable lipid (SM-102): Helper Lipid (DSPC): Cholesterol: DMG-PEG 2000) and mixed with RNA buffer at pH 4.5 at an N:P 5.5 using a glass micromixer chip. After mixing the formulations were dialyzed against PBS (pH 7.4) for 16-24 hours. Formulated LNPs were concentrated using Amicon Ultra™ centrifugal filter devices (EMD Millipore, Billerica, MA) and stored at 5° C. RNA encapsulation was quantified using a Ribogreen™ assay using Triton to disrupt formulated LNPs, all LNPs had 92±9% (N=20) encapsulation. Particle size (87±18 nm Average, N=20), PDI (0.19±0.08 Average, N=20), and Zeta potential (8±5 mV average, N=20) were measured using a Malvern Zetasizer Ultra.

The lipid carriers were each complexed with repRNA encoding mouse IL-12 (SEQ ID NO: 45) according to the methods described in Example 12.

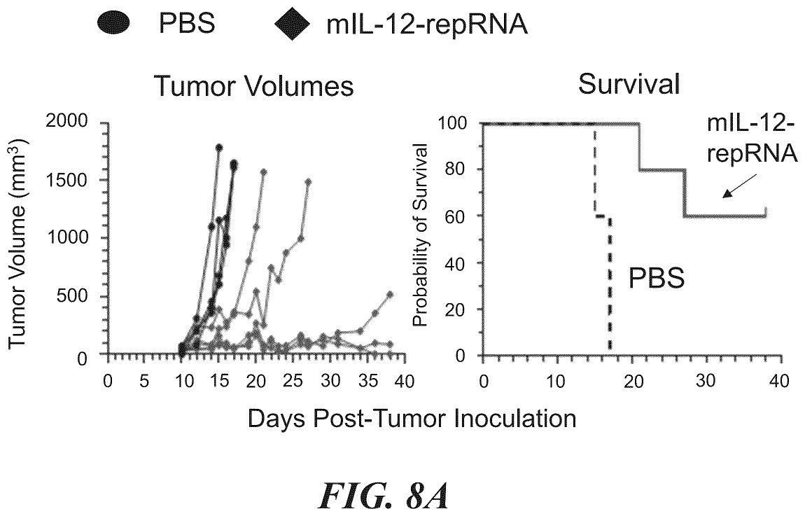

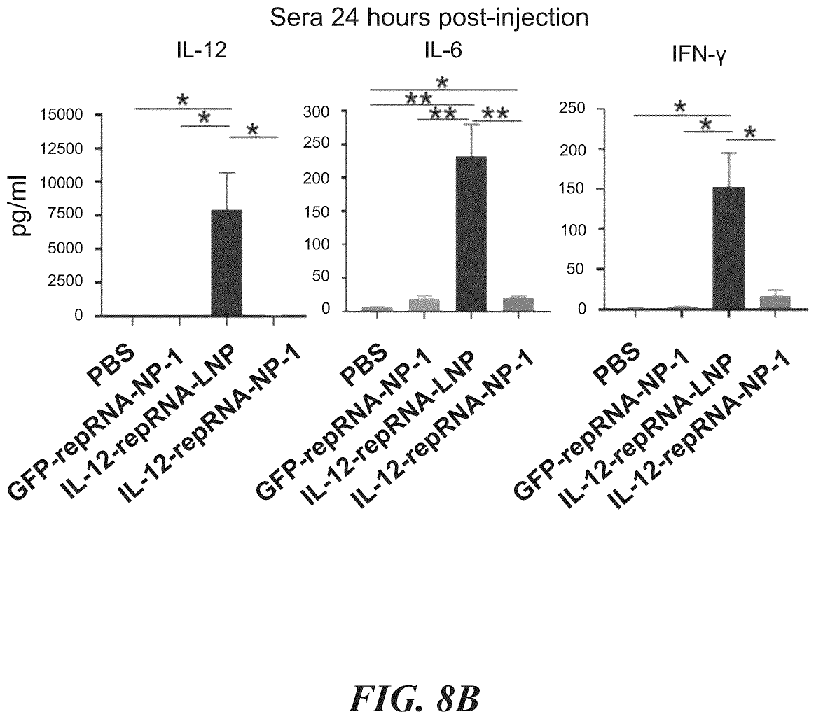

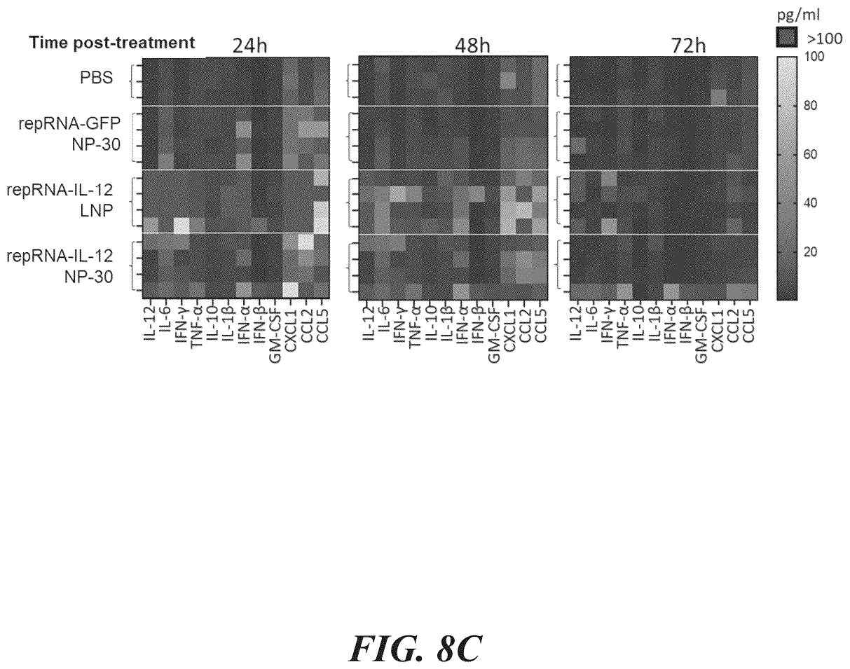

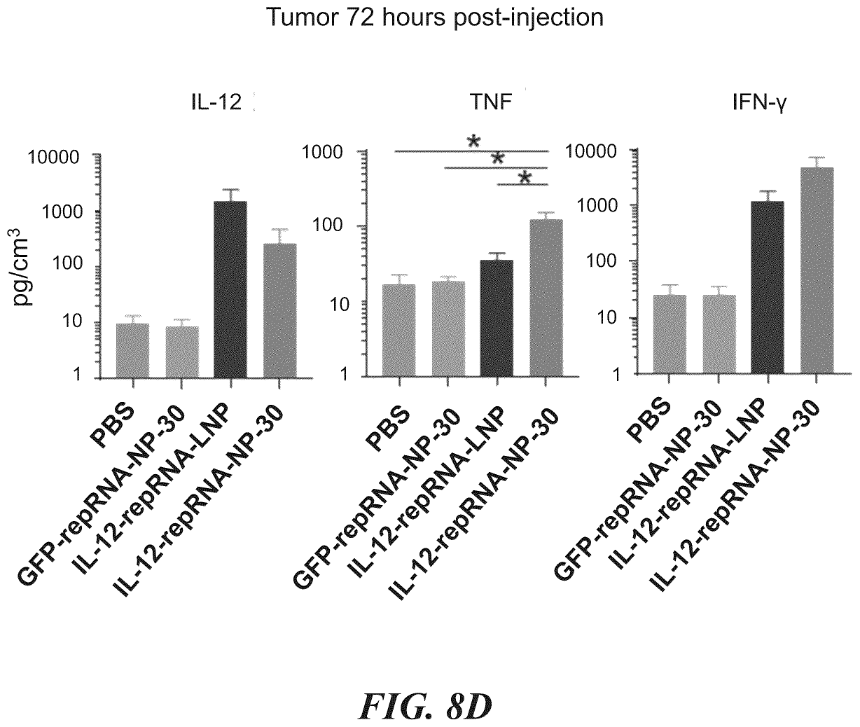

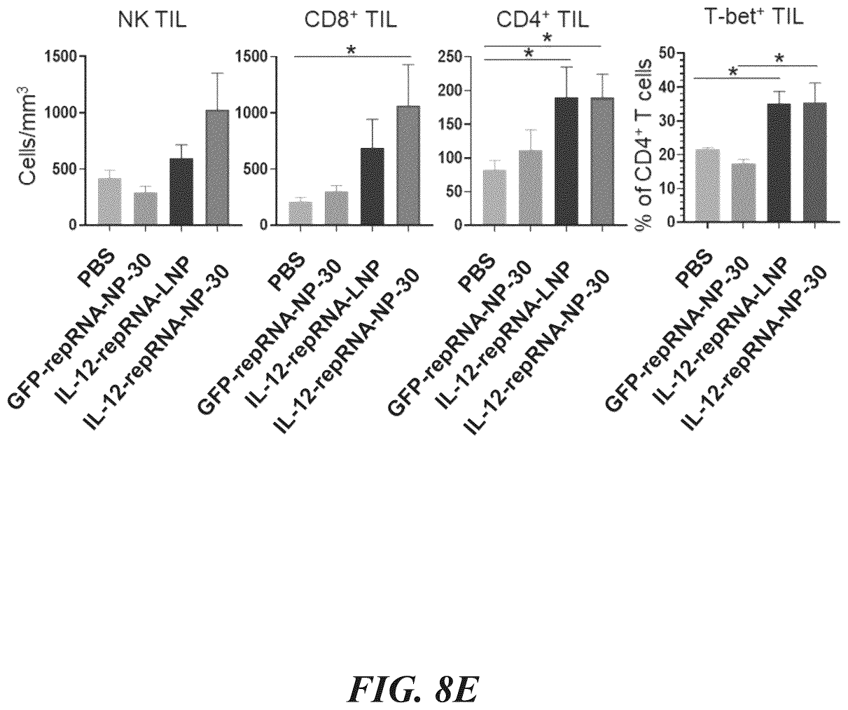

To assess repRNA-IL-12 as a cancer therapeutic, C57BL6 mice were inoculated with 2×10⁵ B16 tumor cells subcutaneously in the hind flank. Once tumors were established, mice were injected with NP-1 complexed with repRNA IL-12 or PBS treatments intratumorally with ultrafine gauge needles in 30 μl total volume. Tumor volumes and survival of mice with established SC B16 melanoma tumors that were injected intratumorally with PBS or with 5 μg IL-12-repRNA-NP-1 10 days post tumor-inoculation are shown in FIG. 8A.

In a separate assay to assess the effect of complexing repRNA-IL-12 with NP-30 and LNPs, B16 tumor-bearing mice were injected intratumorally on day 12 with either PBS, GFP-repRNA-NP-30 as a vehicle control, or 3 μg mIL-12-repRNA formulated with LNP or NP-30. Mice were bled 24 hours post-intratumoral injections and sacrificed 72 hours post-injection on day 15. Sera and tumors were processed and analyzed for extracellular cytokines in sera (FIG. 8B), and tumors (FIG. 8D) determined by LEGENDPLEX™ (Biolegend, San Diego, California, USA) multiplex cytokine capture assay. Tumor lymphocyte infiltration and CD4 Th1 polarization were assessed via intranuclear staining and flow cytometry (FIG. 8D).

### Results:

The data in FIGS. 8A-8E shows that repRNA encoding murine IL-12 leads to a significant delay in tumor growth compared to identical repRNA encapsulated via LNP (FIG. 8A). In addition, mice injected with repRNA-IL-12 formulated with NP-1 had significantly lower levels of pro-inflammatory cytokines IL-12, interleukin-6 (IL-6) and interferon alpha (IFN-γ) compared to mice that received equal doses of the same IL-12 repRNA encapsulated with LNP, and irrelevant repRNA encoding for green fluorescent protein (GFP) formulated with NP-1 as vehicle controls. Additionally, repRNA-IL-12 LNP treated mice displayed increased levels of proinflammatory cytokines and chemokines including IFN-α, CXCL1, CCL2, CCL5 compared to repRNA-IL-12-NP-30 treated mice (FIG. 8C). Tumor lysates from mice treated with both repRNA-IL-12-LNP and repRNA-IL-12-NP-30 displayed elevated levels of intratumoral IL-12, TNF-α (TNF), and IFN-γ compared to PBS and vehicle control groups (FIG. 8D). This data shows that biologically active IL-12 is effectively expressed from the replicon RNA when delivered via LNP or the nanoparticles tested (NP-1 and NP-30). Furthermore, tumors treated with repRNA-IL-12 formulated with NP-30 or LNP showed similar increases in anti-tumor immune cell types including natural killer (NK) cells, CD8 T cells, and TH1-polarized CD4 T cells that are desirable for effective anti-tumor responses (as determined by the pro-T_{H}1 transcription factor T_{bet}) (FIG. 8E)

Interestingly, matched serum cytokine analysis supports that the transfection of cells via NP-30 is highly localized and retained in the tumor microenvironment (TME) as indicated by minimal serum cytokines in the repRNA-IL-12-NP-30 treated mice, whereas elevated levels of inflammatory cytokines and chemokines in the repRNA-IL-12-LNP treated groups suggest that the LNP transfection is widely distributed and are indicative of clinical immune related adverse events (irAEs) and systemic toxicity.

### Example 19: IL-12 Replicon RNA Efficacy in a Syngeneic Mouse Model for Breast Cancer

To assess repRNA-IL-12 as a cancer therapeutic, lipid nanoparticles were prepared and complexed with repRNA-IL-12. NP-30 and LNP were prepared according to Example 18 for the assays described below.

A 4T1 mouse model was generated for the assays described in this Example. The 4T1 mammary carcinoma is a transplantable tumor cell line that is highly tumorigenic and invasive and, unlike most tumor models, can spontaneously metastasize from the primary tumor in the mammary gland to multiple distant sites including lymph nodes, blood, liver, lung, brain, and bone.

4T1 tumor cells (0.5×10⁶) mixed 1:1 with MATRIGEL™ (solubilized basement membrane preparation extracted from the Engelbreth-Holm-Swarm (EHS) mouse sarcoma, Corning, Inc., Corning, NY, USA) were implanted into the 4′ mammary fat pad of 6 to 8-week old female BALB/c mice. Tumor measurements for all assays were performed via digital caliper in single-blind studies where the treatments were unknown to the individual taking the tumor measurements.

Treatment of tumor-bearing mice were performed once tumors were established (˜250 mm³). Briefly, mice were normally distributed into groups based off tumor volumes and treated with either PBS, or 3 ug repRNA encoding for murine IL-12p40 linked to murine IL-12p35 subunits to form biologically active IL-12p70 (the heterodimer of the p40 and p35 subunits) either encapsulated with either LNP or NP-30 (without iron) as described above. Treatments were administered via intratumoral injection with ultra-fine 30 g needles in 30 μl total volume. Mice were bled 24 hours post-intratumoral injection and serum was assessed for pro-inflammatory cytokines via LEGENDPLEX™ assay (BioLegend) and acquisition on a BD Symphony flow cytometer.

### Results:

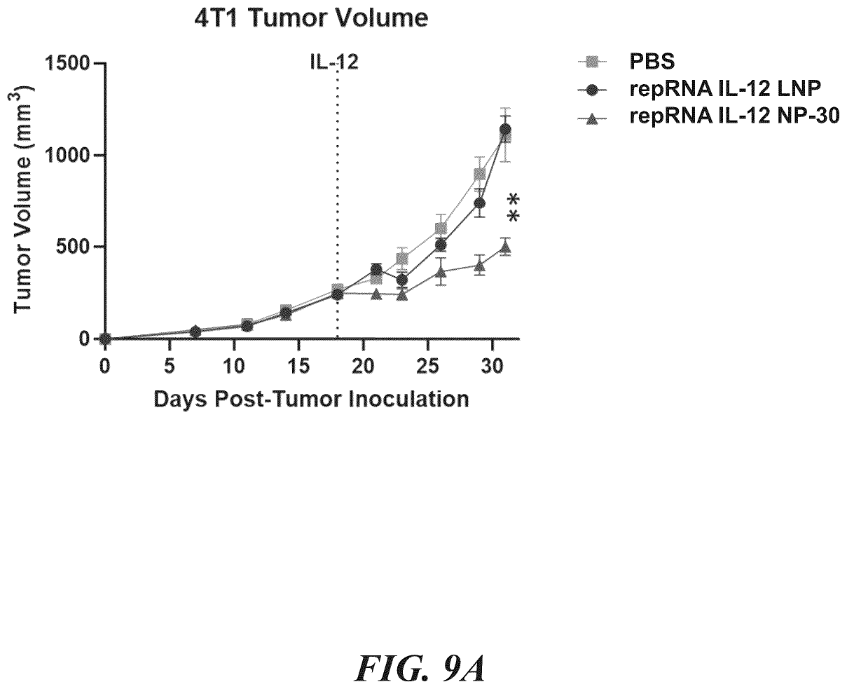

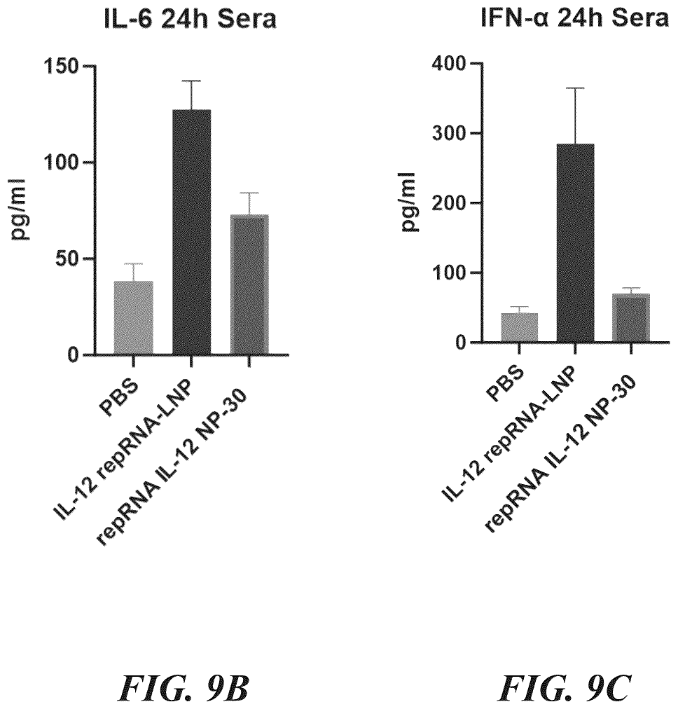

The data in FIGS. 9A-9C show that repRNA encoding murine IL-12 leads to a significant delay in tumor growth compared to identical repRNA encapsulated via LNP (FIG. 9A). In addition, mice injected with repRNA-IL-12 formulated with NP-30 had significantly lower levels of pro-inflammatory cytokines interleukin-6 (IL-6) and interferon alpha (IFN-α) compared to mice that received equal doses of the same repRNA encapsulated with LNP (FIGS. 9B-9C). Collectively, this data supports that NP-30 enabled repRNA expression in a highly localized manner proximal to the injection site (intratumorally), while the LNP formulation led to distal expression of repRNAs leading to systemic inflammatory cytokines commonly associated with cytokine response syndrome (CRS). The prolonged tumor control displayed by the repRNA-IL-12-NP-30 group compared to the repRNA-IL-12-LNP group also shows that repRNA-mediated IL-12 expression was extended, and/or expressed at greater local concentrations when delivered with NP-30 compared to LNP.

### SEQUENCES

Human IL-12A (p35 subunit) Amino Acid Sequence-NP_000873.2
interleukin-12 subunit alpha isoform 1 precursor
[Homo sapiens]
SEQ ID NO: 1:
MWPPGSASQPPPSPAAATGLHPAARPVSLQCRLSMCPARSLLLVATLVLLDHLSLARNLPVATPDPGMFPCLHHSQN
LLRAVSNMLQKARQTLEFYPCTSEEIDHEDITKDKTSTVEACLPLELTKNESCLNSRETSFITNGSCLASRKTSFMM
ALCLSSIYEDLKMYQVEFKTMNAKLLMDPKRQIFLDQNMLAVIDELMQALNFNSETVPQKSSLEEPDFYKTKIKLCI
LLHAFRIRAVTIDRVMSYLNAS
Human IL-12B (p40 subunit) Amino Acid Sequence-NP_002178.2
interleukin-12 subunit beta precursor [Homo sapiens]
SEQ ID NO: 2:
MCHQQLVISWFSLVFLASPLVAIWELKKDVYVVELDWYPDAPGEMVVLTCDTPEEDGITWTLDQSSEVLGSGKTLTI
QVKEFGDAGQYTCHKGGEVLSHSLLLLHKKEDGIWSTDILKDQKEPKNKTFLRCEAKNYSGRFTCWWLTTISTDLTF
SVKSSRGSSDPQGVTCGAATLSAERVRGDNKEYEYSVECQEDSACPAAEESLPIEVMVDAVHKLKYENYTSSFFIRD
IIKPDPPKNLQLKPLKNSRQVEVSWEYPDTWSTPHSYFSLTFCVQVQGKSKREKKDRVFTDKTSATVICRKNASISV
RAQDRYYSSSWSEWASVPCS
Mouse IL-12a Amino Acid Sequence-NP_001152896.1
interleukin-12 subunit alpha isoform 1
[Mus musculus]
SEQ ID NO: 3:
MVSVPTASPSASSSSSQCRSSMCQSRYLLFLATLALLNHLSLARVIPVSGPARCLSQSRNLLKTTDDMVKTAREKLK
HYSCTAEDIDHEDITRDQTSTLKTCLPLELHKNESCLATRETSSTTRGSCLPPQKTSLMMTLCLGSIYEDLKMYQTE
FQAINAALQNHNHQQIILDKGMLVAIDELMQSLNHNGETLRQKPPVGEADPYRVKMKLCILLHAFSTRVVTINRVMG
YLSSA
Mouse IL-12b Amino Acid Sequence-NP_001290173.1
interleukin-12 subunit beta precursor
[Mus musculus]
SEQ ID NO: 4
MCPQKLTISWFAIVLLVSPLMAMWELEKDVYVVEVDWTPDAPGETVNLTCDTPEEDDITWTSDQRHGVIGSGKTLTI
TVKEFLDAGQYTCHKGGETLSHSHLLLHKKENGIWSTEILKNFKNKTFLKCEAPNYSGRFTCSWLVQRNMDLKFNIK
SSSSSPDSRAVTCGMASLSAEKVTLDQRDYEKYSVSCQEDVTCPTAEETLPIELALEARQQNKYENYSTSFFIRDII
KPDPPKNLQMKPLKNSQVEVSWEYPDSWSTPHSYFSLKFFVRIQRKKEKMKETEEGCNQKGAFLVEKTSTEVQCKGG
NVCVQAQDRYYNSSCSKWACVPCRVRS
Human IL-12 p40-elastin-p35 Fusion Protein Amino
Acid Sequence
SEQ ID NO: 5
MCHQQLVISWFSLVFLASPLVAIWELKKDVYVVELDWYPDAPGEMVVLTCDTPEEDGITWTLDQSSEVLGSGKTLTI
QVKEFGDAGQYTCHKGGEVLSHSLLLLHKKEDGIWSTDILKDQKEPKNKTFLRCEAKNYSGRFTCWWLTTISTDLTF
SVKSSRGSSDPQGVTCGAATLSAERVRGDNKEYEYSVECQEDSACPAAEESLPIEVMVDAVHKLKYENYTSSFFIRD
IIKPDPPKNLQLKPLKNSRQVEVSWEYPDTWSTPHSYFSLTFCVQVQGKSKREKKDRVFTDKTSATVICRKNASISV
RAQDRYYSSSWSEWASVPCSVPGVGVPGVGARNLPVATPDPGMFPCLHHSQNLLRAVSNMLQKARQTLEFYPCTSEE
IDHEDITKDKTSTVEACLPLELTKNESLNSRETSFITNGSCLASRKTSFMMALCLSSIYEDLKMYQVEFKTMNAKLL
MDPKRQIFLDQNMLAVIDELMQALNFNSETVPQKSSLEEPDFYKTKIKLILLHAFRIRAVTIDRVMSYLNAS
Mouse IL-12 p40-Elastin-p35 Fusion Protein Amino
Acid Sequence
SEQ ID NO: 6:
MCPQKLTISWFAIVLLVSPLMAMWELEKDVYVVEVDWTPDAPGETVNLTCDTPEEDDITWTSDQRHGVIGSGKTLTI
TVKEFLDAGQYTCHKGGETLSHSHLLLHKKENGIWSTEILKNFKNKTFLKCEAPNYSGRFTCSWLVQRNMDLKFNIK
SSSSSPDSRAVTCGMASLSAEKVTLDQRDYEKYSVSCQEDVTCPTAEETLPIELALEARQQNKYENYSTSFFIRDII
KPDPPKNLQMKPLKNSQVEVSWEYPDSWSTPHSYFSLKFFVRIQRKKEKMKETEEGCNQKGAFLVEKTSTEVQCKGG
NVCVQAQDRYYNSSCSKWACVPCRVRSVPGVGVPGVGRVIPVSGPARCLSQSRNLLKTTDDMVKTAREKLKHYSCTA
EDIDHEDITRDQTSTLKTCLPLELHKNESCLATRETSSTTRGSCLPPQKTSLMMTLCLGSIYEDLKMYQTEFQAINA
ALQNHNHQQIILDKGMLVAIDELMQSLNHNGETLRQKPPVGEADPYRVKMKLCILLHAFSTRVVTINRVMGYLSSA
Human IFNγ Amino Acid Sequence-NP_000610.2 interferon
gamma precursor [Homo sapiens]
SEQ ID NO: 7:
MKYTSYILAFQLCIVLGSLGCYCQDPYVKEAENLKKYFNAGHSDVADNGTLFLGILKNWKEESDRKIMQSQIVSFYF
KLFKNFKDDQSIQKSVETIKEDMNVKFFNSNKKKRDDFEKLTNYSVTDLNVQRKAIHELIQVMAELSPAAKTGKRKR
SQMLFRGRRASQ
Mouse IFNγ Amino Acid Sequence-NP_032363.1 interferon
gamma precursor [Mus musculus]
SEQ ID NO: 8:
MNATHCILALQLFLMAVSGCYCHGTVIESLESLNNYFNSSGIDVEEKSLFLDIWRNWQKDGDMKILQSQIISFYLRL
FEVLKDNQAISNNISVIESHLITTFFSNSKAKKDAFMSIAKFEVNNPQVQRQAFNELIRVVHQLLPESSLRKRKRSR
C
Human IL-2 Amino Acid Sequence-NP_000577.2
interleukin-2 precursor [Homo sapiens]
SEQ ID NO: 9:
MYRMQLLSCIALSLALVTNSAPTSSSTKKTQLQLEHLLLDLQMILNGINNYKNPKLTRMLTFKFYMPKKATELKHLQ
CLEEELKPLEEVLNLAQSKNFHLRPRDLISNINVIVLELKGSETTFMCEYADETATIVEFLNRWITFCQSIISTLT
Human IL-15 Amino Acid Sequence-NP_000576.1
interleukin-15 isoform 1 preproprotein
[Homo sapiens]
SEQ ID NO: 10:
MRISKPHLRSISIQCYLCLLLNSHFLTEAGIHVFILGCFSAGLPKTEANWVNVISDLKKIEDLIQSMHIDATLYTES
DVHPSCKVTAMKCFLLELQVISLESGDASIHDTVENLIILANNSLSSNGNVTESGCKECEELEEKNIKEFLQSFVHI
VOMFINTS
Human IL-18 Amino Acid Sequence-NP_001373349.1
interleukin-18 isoform 1 [Homo sapiens]
SEQ ID NO: 11:
MAAEPVEDNCINFVAMKFIDNTLYFIAEDDENLESDYFGKLESKLSVIRNLNDQVLFIDQGNRPLFEDMTDSDCRDN
APRTIFIISMYKDSQPRGMAVTISVKCEKISTLSCENKIISFKEMNPPDNIKDTKSDIIFFQRSVPGHDNKMQFESS
SYEGYFLACEKERDLFKLILKKEDELGDRSIMFTVQNED
Human IL-21 Amino Acid Sequence-NP_068575.1 interleukin-21
isoform 1 precursor [Homo sapiens]
SEQ ID NO: 12:
MRSSPGNMERIVICLMVIFLGTLVHKSSSQGQDRHMIRMRQLIDIVDQLKNYVNDLVPEFLPAPEDVETNCEWSAFS
CFQKAQLKSANTGNNERIINVSIKKLKRKPPSTNAGRRQKHRLTCPSCDSYEKKPPKEFLERFKSLLQKMIHQHLSS
RTHGSEDS
Human IL-23A Amino Acid Sequence-NP_057668.1 interleukin-23
subunit alpha precursor [Homo sapiens]
SEQ ID NO: 13:
MLGSRAVMLLLLLPWTAQGRAVPGGSSPAWTQCQQLSQKLCTLAWSAHPLVGHMDLREEGDEETTNDVPHIQCGDGC
DPQGLRDNSQFCLQRIHQGLIFYEKLLGSDIFTGEPSLLPDSPVGQLHASLLGLSQLLQPEGHHWETQQIPSLSPSQ
PWQRLLLRFKILRSLQAFVAVAARVFAHGAATLSP
Human IL-27A Amino Acid Sequence-NP_663634.2 interleukin-27
subunit alpha precursor [Homo sapiens]
SEQ ID NO: 14:
MGQTAGDLGWRLSLLLLPLLLVQAGVWGFPRPPGRPQLSLQELRREFTVSLHLARKLLSEVRGQAHRFAESHLPGVN
LYLLPLGEQLPDVSLTFQAWRRLSDPERLCFISTTLQPFHALLGGLGTQGRWTNMERMQLWAMRLDLRDLQRHLRFQ
VLAAGFNLPEEEEEEEEEEEEERKGLLPGALGSALQGPAQVSWPQLLSTYRLLHSLELVLSRAVRELLLLSKAGHSV
WPLGFPTLSPQP
Human IL-27B Amino Acid Sequence-NP_005746.2 interleukin-27
subunit beta precursor [Homo sapiens]
SEQ ID NO: 15:
MTPQLLLALVLWASCPPCSGRKGPPAALTLPRVQCRASRYPIAVDCSWTLPPAPNSTSPVSFIATYRLGMAARGHSW
PCLQQTPTSTSCTITDVQLFSMAPYVLNVTAVHPWGSSSSFVPFITEHIIKPDPPEGVRLSPLAERQLQVQWEPPGS
WPFPEIFSLKYWIRYKRQGAARFHRVGPIEATSFILRAVRPRARYYVQVAAQDLTDYGELSDWSLPATATMSLGK
RNA encoding a mouse IL-12 fusion of p40-Elastin-p35
SEQ ID NO: 16:
CUGAGAUCACCGGGACGCGUCAACAUGUGUCCUCAGAAGCUAACCAUCUCCUGGUUUGCCAUCGUUUUGCUGGUGUC
UCCACUCAUGGCCAUGUGGGAGCUGGAGAAAGACGUUUAUGUUGUAGAGGUGGACUGGACUCCCGAUGCCCCUGGAG
AAACAGUGAACCUCACCUGUGACACGCCUGAAGAAGAUGACAUCACCUGGACCUCAGACCAGAGACAUGGAGUCAUA
GGCUCUGGAAAGACCCUGACCAUCACUGUCAAAGAGUUUCUAGAUGCUGGCCAGUACACCUGCCACAAAGGAGGCGA
GACUCUGAGCCACUCACAUCUGCUGCUCCACAAGAAGGAAAAUGGAAUUUGGUCCACUGAAAUUUUAAAAAAUUUCA
AAAACAAGACUUUCCUGAAGUGUGAAGCACCAAAUUACUCCGGACGGUUCACGUGCUCAUGGCUGGUGCAAAGAAAC
AUGGACUUGAAGUUCAACAUCAAGAGCAGUAGCAGUUCCCCUGACUCUCGGGCAGUGACAUGUGGAAUGGCGUCUCU
GUCUGCAGAGAAGGUCACACUGGACCAAAGGGACUAUGAGAAGUAUUCAGUGUCCUGCCAGGAGGAUGUCACCUGCC
CAACUGCCGAGGAGACCCUGCCCAUUGAACUGGCGUUGGAAGCACGGCAGCAGAAUAAAUAUGAGAACUACAGCACC
AGCUUCUUCAUCAGGGACAUCAUCAAACCAGACCCGCCCAAGAACUUGCAGAUGAAGCCUUUGAAGAACUCACAGGU
GGAGGUCAGCUGGGAGUACCCUGACUCCUGGAGCACUCCCCAUUCCUACUUCUCCCUCAAGUUCUUUGUUCGAAUCC
AGCGCAAGAAAGAAAAGAUGAAGGAGACAGAGGAGGGGUGUAACCAGAAAGGUGCGUUCCUCGUAGAGAAGACAUCU
ACCGAAGUCCAAUGCAAAGGCGGGAAUGUCUGCGUGCAAGCUCAGGAUCGCUAUUACAAUUCCUCAUGCAGCAAGUG
GGCAUGUGUUCCCUGCAGAGUCCGAUCGGUUCCUGGAGUAGGGGUACCUGGAGUGGGCAGGGUCAUACCGGUCUCUG
GACCUGCCAGGUGUCUUAGCCAGUCCCGAAACCUGCUGAAGACCACAGAUGACAUGGUGAAGACGGCCAGAGAAAAG
CUGAAACAUUAUUCCUGCACUGCUGAAGACAUCGAUCAUGAAGACAUCACACGGGACCAAACCAGCACAUUGAAGAC
CUGUUUACCACUGGAACUACACAAGAACGAGAGUUGCCUGGCUACUAGAGAGACUUCUUCCACAACAAGAGGGAGCU
GCCUGCCCCCACAGAAGACGUCUUUGAUGAUGACCCUGUGCCUUGGUAGCAUCUAUGAGGACUUGAAGAUGUACCAG
ACAGAGUUCCAGGCCAUCAACGCAGCACUUCAGAAUCACAACCAUCAGCAGAUCAUUCUAGACAAGGGCAUGCUGGU
GGCCAUCGAUGAGCUGAUGCAGUCUCUGAAUCAUAAUGGCGAGACUCUGCGCCAGAAACCUCCUGUGGGAGAAGCAG
ACCCUUACAGAGUGAAAAUGAAGCUCUGCAUCCUGCUUCACGCCUUCAGCACCCGCGUCGUGACCAUCAACAGGGUG
AUGGGCUAUCUGAGCUCCGCCUAAAG
SEQ ID NOS: 17-27:
See Table 2
Full-Length VEEV RNA Sequence
SEQ ID NO: 28
AUAGGCGGCGCAUGAGAGAAGCCCAGACCAAUUACCUACCCAAAAUGGAGAAAGUUCACGUUGACAUCGAGGAAGAC
AGCCCAUUCCUCAGAGCUUUGCAGCGGAGCUUCCCGCAGUUUGAGGUAGAAGCCAAGCAGGUCACUGAUAAUGACCA
UGCUAAUGCCAGAGCGUUUUCGCAUCUGGCUUCAAAACUGAUCGAAACGGAGGUGGACCCAUCCGACACGAUCCUUG
ACAUUGGAAGUGCGCCCGCCCGCAGAAUGUAUUCUAAGCACAAGUAUCAUUGUAUCUGUCCGAUGAGAUGUGCGGAA
GAUCCGGACAGAUUGUAUAAGUAUGCAACUAAGCUGAAGAAAAACUGUAAGGAAAUAACUGAUAAGGAAUUGGACAA
GAAAAUGAAGGAGCUGGCCGCCGUCAUGAGCGACCCUGACCUGGAAACUGAGACUAUGUGCCUCCACGACGACGAGU
CGUGUCGCUACGAAGGGCAAGUCGCUGUUUACCAGGAUGUAUACGCGGUUGACGGACCGACAAGUCUCUAUCACCAA
GCCAAUAAGGGAGUUAGAGUCGCCUACUGGAUAGGCUUUGACACCACCCCUUUUAUGUUUAAGAACUUGGCUGGAGC
AUAUCCAUCAUACUCUACCAACUGGGCCGACGAAACCGUGUUAACGGCUCGUAACAUAGGCCUAUGCAGCUCUGACG
UUAUGGAGCGGUCACGUAGAGGGAUGUCCAUUCUUAGAAAGAAGUAUUUGAAACCAUCCAACAAUGUUCUAUUCUCU
GUUGGCUCGACCAUCUACCACGAGAAGAGGGACUUACUGAGGAGCUGGCACCUGCCGUCUGUAUUUCACUUACGUGG
CAAGCAAAAUUACACAUGUCGGUGUGAGACUAUAGUUAGUUGCGACGGGUACGUCGUUAAAAGAAUAGCUAUCAGUC
CAGGCCUGUAUGGGAAGCCUUCAGGCUAUGCUGCUACGAUGCACCGCGAGGGAUUCUUGUGCUGCAAAGUGACAGAC
ACAUUGAACGGGGAGAGGGUCUCUUUUCCCGUGUGCACGUAUGUGCCAGCUACAUUGUGUGACCAAAUGACUGGCAU
ACUGGCAACAGAUGUCAGUGCGGACGACGCGCAAAAACUGCUGGUUGGGCUCAACCAGCGUAUAGUCGUCAACGGUC
GCACCCAGAGAAACACCAAUACCAUGAAAAAUUACCUUUUGCCCGUAGUGGCCCAGGCAUUUGCUAGGUGGGCAAAG
GAAUAUAAGGAAGAUCAAGAAGAUGAAAGGCCACUAGGACUACGAGAUAGACAGUUAGUCAUGGGGUGUUGUUGGGC
UUUUAGAAGGCACAAGAUAACAUCUAUUUAUAAGCGCCCGGAUACCCAAACCAUCAUCAAAGUGAACAGCGAUUUCC
ACUCAUUCGUGCUGCCCAGGAUAGGCAGUAACACAUUGGAGAUCGGGCUGAGAACAAGAAUCAGGAAAAUGUUAGAG
GAGCACAAGGAGCCGUCACCUCUCAUUACCGCCGAGGACGUACAAGAAGCUAAGUGCGCAGCCGAUGAGGCUAAGGA
GGUGCGUGAAGCCGAGGAGUUGCGCGCAGCUCUACCACCUUUGGCAGCUGAUGUUGAGGAGCCCACUCUGGAGGCAG
ACGUCGACUUGAUGUUACAAGAGGCUGGGGCCGGCUCAGUGGAGACACCUCGUGGCUUGAUAAAGGUUACCAGCUAC
GAUGGCGAGGACAAGAUCGGCUCUUACGCUGUGCUUUCUCCGCAGGCUGUACUCAAGAGUGAAAAAUUAUCUUGCAU
CCACCCUCUCGCUGAACAAGUCAUAGUGAUAACACACUCUGGCCGAAAAGGGCGUUAUGCCGUGGAACCAUACCAUG
GUAAAGUAGUGGUGCCAGAGGGACAUGCAAUACCCGUCCAGGACUUUCAAGCUCUGAGUGAAAGUGCCACCAUUGUG
UACAACGAACGUGAGUUCGUAAACAGGUACCUGCACCAUAUUGCCACACAUGGAGGAGCGCUGAACACUGAUGAAGA
AUAUUACAAAACUGUCAAGCCCAGCGAGCACGACGGCGAAUACCUGUACGACAUCGACAGGAAACAGUGCGUCAAGA
AAGAACUAGUCACUGGGCUAGGGCUCACAGGCGAGCUGGUGGAUCCUCCCUUCCAUGAAUUCGCCUACGAGAGUCUG
AGAACACGACCAGCCGCUCCUUACCAAGUACCAACCAUAGGGGUGUAUGGCGUGCCAGGAUCAGGCAAGUCUGGCAU
CAUUAAAAGCGCAGUCACCAAAAAAGAUCUAGUGGUGAGCGCCAAGAAAGAAAACUGUGCAGAAAUUAUAAGGGACG
UCAAGAAAAUGAAAGGGCUGGACGUCAAUGCCAGAACUGUGGACUCAGUGCUCUUGAAUGGAUGCAAACACCCCGUA
GAGACCCUGUAUAUUGACGAAGCUUUUGCUUGUCAUGCAGGUACUCUCAGAGCGCUCAUAGCCAUUAUAAGACCUAA
AAAGGCAGUGCUCUGCGGGGAUCCCAAACAGUGCGGUUUUUUUAACAUGAUGUGCCUGAAAGUGCAUUUUAACCACG
AGAUUUGCACACAAGUCUUCCACAAAAGCAUCUCUCGCCGUUGCACUAAAUCUGUGACUUCGGUCGUCUCAACCUUG
UUUUACGACAAAAAAAUGAGAACGACGAAUCCGAAAGAGACUAAGAUUGUGAUUGACACUACCGGCAGUACCAAACC
UAAGCAGGACGAUCUCAUUCUCACUUGUUUCAGAGGGUGGGUGAAGCAGUUGCAAAUAGAUUACAAAGGCAACGAAA
UAAUGACGGCAGCUGCCUCUCAAGGGCUGACCCGUAAAGGUGUGUAUGCCGUUCGGUACAAGGUGAAUGAAAAUCCU
CUGUACGCACCCACCUCAGAACAUGUGAACGUCCUACUGACCCGCACGGAGGACCGCAUCGUGUGGAAAACACUAGC
CGGCGACCCAUGGAUAAAAACACUGACUGCCAAGUACCCUGGGAAUUUCACUGCCACGAUAGAGGAGUGGCAAGCAG
AGCAUGAUGCCAUCAUGAGGCACAUCUUGGAGAGACCGGACCCUACCGACGUCUUCCAGAAUAAGGCAAACGUGUGU
UGGGCCAAGGCUUUAGUGCCGGUGCUGAAGACCGCUGGCAUAGACAUGACCACUGAACAAUGGAACACUGUGGAUUA
UUUUGAAACGGACAAAGCUCACUCAGCAGAGAUAGUAUUGAACCAACUAUGCGUGAGGUUCUUUGGACUCGAUCUGG
ACUCCGGUCUAUUUUCUGCACCCACUGUUCCGUUAUCCAUUAGGAAUAAUCACUGGGAUAACUCCCCGUCGCCUAAC
AUGUACGGGCUGAAUAAAGAAGUGGUCCGUCAGCUCUCUCGCAGGUACCCACAACUGCCUCGGGCAGUUGCCACUGG
AAGAGUCUAUGACAUGAACACUGGUACACUGCGCAAUUAUGAUCCGCGCAUAAACCUAGUACCUGUAAACAGAAGAC
UGCCUCAUGCUUUAGUCCUCCACCAUAAUGAACACCCACAGAGUGACUUUUCUUCAUUCGUCAGCAAAUUGAAGGGC
AGAACUGUCCUGGUGGUCGGGGAAAAGUUGUCCGUCCCAGGCAAAAUGGUUGACUGGUUGUCAGACCGGCCUGAGGC
UACCUUCAGAGCUCGGCUGGAUUUAGGCAUCCCAGGUGAUGUGCCCAAAUAUGACAUAAUAUUUGUUAAUGUGAGGA
CCCCAUAUAAAUACCAUCACUAUCAGCAGUGUGAAGACCAUGCCAUUAAGCUUAGCAUGUUGACCAAGAAAGCUUGU
CUGCAUCUGAAUCCCGGCGGAACCUGUGUCAGCAUAGGUUAUGGUUACGCUGACAGGGCCAGCGAAAGCAUCAUUGG
UGCUAUAGCGCGGCAGUUCAAGUUUUCCCGGGUAUGCAAACCGAAAUCCUCACUUGAAGAGACGGAAGUUCUGUUUG
UAUUCAUUGGGUACGAUCGCAAGGCCCGUACGCACAAUCCUUACAAGCUUUCAUCAACCUUGACCAACAUUUAUACA
GGUUCCAGACUCCACGAAGCCGGAUGUGCACCCUCAUAUCAUGUGGUGCGAGGGGAUAUUGCCACGGCCACCGAAGG
AGUGAUUAUAAAUGCUGCUAACAGCAAAGGACAACCUGGCGGAGGGGUGUGCGGAGCGCUGUAUAAGAAAUUCCCGG
AAAGCUUCGAUUUACAGCCGAUCGAAGUAGGAAAAGCGCGACUGGUCAAAGGUGCAGCUAAACAUAUCAUUCAUGCC
GUAGGACCAAACUUCAACAAAGUUUCGGAGGUUGAAGGUGACAAACAGUUGGCAGAGGCUUAUGAGUCCAUCGCUAA
GAUUGUCAACGAUAACAAUUACAAGUCAGUAGCGAUUCCACUGUUGUCCACCGGCAUCUUUUCCGGGAACAAAGAUC
GACUAACCCAAUCAUUGAACCAUUUGCUGACAGCUUUAGACACCACUGAUGCAGAUGUAGCCAUAUACUGCAGGGAC
AAGAAAUGGGAAAUGACUCUCAAGGAAGCAGUGGCUAGGAGAGAAGCAGUGGAGGAGAUAUGCAUAUCCGACGACUC
UUCAGUGACAGAACCUGAUGCAGAGCUGGUGAGGGUGCAUCCGAAGAGUUCUUUGGCUGGAAGGAAGGGCUACAGCA
CAAGCGAUGGCAAAACUUUCUCAUAUUUGGAAGGGACCAAGUUUCACCAGGCGGCCAAGGAUAUAGCAGAAAUUAAU
GCCAUGUGGCCCGUUGCAACGGAGGCCAAUGAGCAGGUAUGCAUGUAUAUCCUCGGAGAAAGCAUGAGCAGUAUUAG
GUCGAAAUGCCCCGUCGAAGAGUCGGAAGCCUCCACACCACCUAGCACGCUGCCUUGCUUGUGCAUCCAUGCCAUGA
CUCCAGAAAGAGUACAGCGCCUAAAAGCCUCACGUCCAGAACAAAUUACUGUGUGCUCAUCCUUUCCAUUGCCGAAG
UAUAGAAUCACUGGUGUGCAGAAGAUCCAAUGCUCCCAGCCUAUAUUGUUCUCACCGAAAGUGCCUGCGUAUAUUCA
UCCAAGGAAGUAUCUCGUGGAAACACCACCGGUAGACGAGACUCCGGAGCCAUCGGCAGAGAACCAAUCCACAGAGG
GGACACCUGAACAACCACCACUUAUAACCGAGGAUGAGACCAGGACUAGAACGCCUGAGCCGAUCAUCAUCGAAGAG
GAAGAAGAGGAUAGCAUAAGUUUGCUGUCAGAUGGCCCGACCCACCAGGUGCUGCAAGUCGAGGCAGACAUUCACGG
GCCGCCCUCUGUAUCUAGCUCAUCCUGGUCCAUUCCUCAUGCAUCCGACUUUGAUGUGGACAGUUUAUCCAUACUUG
ACACCCUGGAGGGAGCUAGCGUGACCAGCGGGGCAACGUCAGCCGAGACUAACUCUUACUUCGCAAAGAGUAUGGAG
UUUCUGGCGCGACCGGUGCCUGCGCCUCGAACAGUAUUCAGGAACCCUCCACAUCCCGCUCCGCGCACAAGAACACC
GUCACUUGCACCCAGCAGGGCCUGCUCGAGAACCAGCCUAGUUUCCACCCCGCCAGGCGUGAAUAGGGUGAUCACUA
GAGAGGAGCUCGAGGCGCUUACCCCGUCACGCACUCCUAGCAGGUCGGUCUCGAGAACCAGCCUGGUCUCCAACCCG
CCAGGCGUAAAUAGGGUGAUUACAAGAGAGGAGUUUGAGGCGUUCGUAGCACAACAACAAUGACGGUUUGAUGCGGG
UGCAUACAUCUUUUCCUCCGACACCGGUCAAGGGCAUUUACAACAAAAAUCAGUAAGGCAAACGGUGCUAUCCGAAG
UGGUGUUGGAGAGGACCGAAUUGGAGAUUUCGUAUGCCCCGCGCCUCGACCAAGAAAAAGAAGAAUUACUACGCAAG
AAAUUACAGUUAAAUCCCACACCUGCUAACAGAAGCAGAUACCAGUCCAGGAAGGUGGAGAACAUGAAAGCCAUAAC
AGCUAGACGUAUUCUGCAAGGCCUAGGGCAUUAUUUGAAGGCAGAAGGAAAAGUGGAGUGCUACCGAACCCUGCAUC
CUGUUCCUUUGUAUUCAUCUAGUGUGAACCGUGCCUUUUCAAGCCCCAAGGUCGCAGUGGAAGCCUGUAACGCCAUG
UUGAAAGAGAACUUUCCGACUGUGGCUUCUUACUGUAUUAUUCCAGAGUACGAUGCCUAUUUGGACAUGGUUGACGG
AGCUUCAUGCUGCUUAGACACUGCCAGUUUUUGCCCUGCAAAGCUGCGCAGCUUUCCAAAGAAACACUCCUAUUUGG
AACCCACAAUACGAUCGGCAGUGCCUUCAGCGAUCCAGAACACGCUCCAGAACGUCCUGGCAGCUGCCACAAAAAGA
AAUUGCAAUGUCACGCAAAUGAGAGAAUUGCCCGUAUUGGAUUCGGCGGCCUUUAAUGUGGAAUGCUUCAAGAAAUA
UGCGUGUAAUAAUGAAUAUUGGGAAACGUUUAAAGAAAACCCCAUCAGGCUUACUGAAGAAAACGUGGUAAAUUACA
UUACCAAAUUAAAAGGACCAAAAGCUGCUGCUCUUUUUGCGAAGACACAUAAUUUGAAUAUGUUGCAGGACAUACCA
AUGGACAGGUUUGUAAUGGACUUAAAGAGAGACGUGAAAGUGACUCCAGGAACAAAACAUACUGAAGAACGGCCCAA
GGUACAGGUGAUCCAGGCUGCCGAUCCGCUAGCAACAGCGUAUCUGUGCGGAAUCCACCGAGAGCUGGUUAGGAGAU
UAAAUGCGGUCCUGCUUCCGAACAUUCAUACACUGUUUGAUAUGUCGGCUGAAGACUUUGACGCUAUUAUAGCCGAG
CACUUCCAGCCUGGGGAUUGUGUUCUGGAAACUGACAUCGCGUCGUUUGAUAAAAGUGAGGACGACGCCAUGGCUCU
GACCGCGUUAAUGAUUCUGGAAGACUUAGGUGUGGACGCAGAGCUGUUGACGCUGAUUGAGGCGGCUUUCGGCGAAA
UUUCAUCAAUACAUUUGCCCACUAAAACUAAAUUUAAAUUCGGAGCCAUGAUGAAAUCUGGAAUGUUCCUCACACUG
UUUGUGAACACAGUCAUUAACAUUGUAAUCGCAAGCAGAGUGUUGAGAGAACGGCUAACCGGAUCACCAUGUGCAGC
AUUCAUUGGAGAUGACAAUAUCGUGAAAGGAGUCAAAUCGGACAAAUUAAUGGCAGACAGGUGCGCCACCUGGUUGA
AUAUGGAAGUCAAGAUUAUAGAUGCUGUGGUGGGCGAGAAAGCGCCUUAUUUCUGUGGAGGGUUUAUUUUGUGUGAC
UCCGUGACCGGCACAGCGUGCCGUGUGGCAGACCCCCUAAAAAGGCUGUUUAAGCUUGGCAAACCUCUGGCAGCAGA
CGAUGAACAUGAUGAUGACAGGAGAAGGGCAUUGCAUGAAGAGUCAACACGCUGGAACCGAGUGGGUAUUCUUUCAG
AGCUGUGCAAGGCAGUAGAAUCAAGGUAUGAAACCGUAGGAACUUCCAUCAUAGUUAUGGCCAUGACUACUCUAGCU
AGCAGUGUUAAAUCAUUCAGCUACCUGAGAGGGGCCCCUAUAACUCUCUACGGCUAACCUGAAUGGACUACGACAUA
GUCUAGUCCGCCAAG
VEEV RNA polymerase Amino Acid Sequence
(NCBI Accession: AXP98866.1)
SEQ ID NO: 29:
RELPVLDSAAFNVECFKKYACNNEYWETFKENPIRLTEENVVNYITKLKGP
VEEV RNA polymerase Amino Acid Sequence
(NCBI Accession: AXP98867.1)
SEQ ID NO: 30:
TQMRELPVLDSAAFNVECFKKYACNNEYWETFKENPIRLTE
Polyprotein Amino Acid Sequence [Venezuelan equine
encephalitis virus](GenBank: ALE15116.1)
SEQ ID NO: 31:
MKAITARRILQGLGHYLKAEGKVECYRTLHPVPLYSSSVNRAFSSPKVAVEACNAMLKENFPTVASYCIIPEYDAYL
DMIDGASCCLDTASFCPAKLRSFPKKHSYLEPTIRSAVPSAIQNTLQNVLAAATKRNCNVTQMRELPVLDSAAFNVE
CFKKYACNNEYWKTFKENPIRLTEENVINYITKLKGPKAAALYAKTHNLNMLQDIPMDRFVMDLKRDVKVTPGTKHT
EERPKVQVIQAADPLATAYLCGIHRELVRRLNAVLLPNIHTLFDMSAEDFDAIIAEHFQPGDCVLETDIASFDKSED
DAMALTAMMILEDLGVDAELLTLIEAAFGEISSIHLPTKTKFKFGAMMKSGMFLTLFVNTVINIVIASRVLRERLTG
SPCAAFIGDDNIVKGVKSDKLMADRCATWLNMEVKIIDAVVGEKAPYFCGGFILCDSVTGTACRVADPLKRLFKLGK
PLAADDEHDDDRRRALHEESTRWNRVGILPELCKAVESRYETVGTSVIVMAMATLASSVKSFSYLRGAPITLYG

The following sequence (SEQ ID NO: 32) is formatted to signify vector backbone and cytokine open reading frames as follows: lower case letter signify the VEEV replicon backbone sequence; UPPER CASE letters signify the mouse IL-12 fusion open reading frame.

SEQ ID NO: 32:
Full Length-VEEV + mouse IL-12 fusion RNA Encoding Sequence
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaauaacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaauaagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucauuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaccggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugcuauuuggacauagguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacauaauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acauucauacacuguuugauaugucggcugaagacuuugacgcuauuauagccgagcacu
uccagccuggggauuguguucuggaaacugacaucgcgucguuugauaaaagugaggacg
acgccauggcucugaccgcguuaaugauucuggaagacuuagguguggacgcagagcugu
ugacgcugauugaggcggcuuucggcgaaauuucaucaauacauuugcccacuaaaacua
aauuuaaauucggagccaugaugaaaucuggaauguuccucacacuguuugugaacacag
ucauuaacauuguaaucgcaagcagaguguugagagaacggcuaaccggaucaccaugug
cagcauucauuggagaugacaauaucgugaaaggagucaaaucggacaaauuaauggcag
acaggugcgccaccugguugaauauggaagucaagauuauagaugcuguggugggcgaga
aagcgccuuauuucuguggaggguuuauuuugugugacuccgugaccggcacagcgugcc
guguggcagacccccuaaaaaggcuguuuaagcuuggcaaaccucuggcagcagacgaug
aacaugaugaugacaggagaagggcauugcaugaagagucaacacgcuggaaccgagugg
guauucuuucagagcugugcaaggcaguagaaucaagguaugaaaccguaggaacuucca
ucauaguuauggccaugacuacucuagcuagcaguguuaaaucauucagcuaccugagag
gggccccuauaacucucuacggcuaaccugaaugcaaugucacgcaaaugagagaauugc
ccguauuggauucggcggccuuuaauguggaaugcuucaagaaauaugcguguaauaaug
aauauugggaaacguuuaaagaaaaccccaucaggcuuacugaagaaaacgugguaaauu
acauuaccaaauuaaaaggaccaaaagcugcugcucuuuuugcgaagacacauaauuuga
auauguugcaggacauaccaauggacagguuuguaauggacuuaaagagagacgugaaag
ugacuccaggaacaaaacauacugaagaacggcccaagguacaggugauccaggcugccg
auccgcuagcaacagcguaucugugcggaauccaccgagagcugguuaggagauuaaaug
cgguccugcuuccgaacauucauacacuguuugauaugucggcugaagacuuugacgcua
uuauagccgagcacuuccagccuggggauuguguucuggaaacugacaucgcgucguuug
auaaaagugaggacgacgccauggcucugaccgcguuaaugauucuggaagacuuaggug
uggacgcagagcuguugacgcugauugaggcggcuuucggcgaaauuucaucaauacauu
ugcccacuaaaacuaaauuuaaauucggagccaugaugaaaucuggaauguuccucacac
uguuugugaacacagucauuaacauuguaaucgcaagcagaguguugagagaacggcuaa
ccggaucaccaugugcagcauucauuggagaugacaauaucgugaaaggagucaaaucgg
acaaauuaauggcagacaggugcgccaccugguugaauauggaagucaagauuauagaug
cuguggugggcgagaaagcgccuuagcuuggcaaaccucuggcagcagacgaugaacaug
augaugacaggagaagggcauugcaugaagagucaacacgcuggaaccgaguggguauuc
uuucagagcugugcaaggcaguagaaucaagguaugaaaccguaggaacuuccaucauag
uuauggccaugacuacucuagcuagcaguguuaaaucauucagcuaccugagaggggccc
cuauaacucucuacggcuaaccugaauggacuacgacauagucuaguccgccaagCUGAG
AUCACCGGGACGCGUCAACAUGUGUCCUCAGAAGCUAACCAUCUCCUGGUUUGCCAUCGU
UUUGCUGGUGUCUCCACUCAUGGCCAUGUGGGAGCUGGAGAAAGACGUUUAUGUUGUAGA
GGUGGACUGGACUCCCGAUGCCCCUGGAGAAACAGUGAACCUCACCUGUGACACGCCUGA
AGAAGAUGACAUCACCUGGACCUCAGACCAGAGACAUGGAGUCAUAGGCUCUGGAAAGAC
CCUGACCAUCACUGUCAAAGAGUUUCUAGAUGCUGGCCAGUACACCUGCCACAAAGGAGG
CGAGACUCUGAGCCACUCACAUCUGCUGCUCCACAAGAAGGAAAAUGGAAUUUGGUCCAC
UGAAAUUUUAAAAAAUUUCAAAAACAAGACUUUCCUGAAGUGUGAAGCACCAAAUUACUC
CGGACGGUUCACGUGCUCAUGGCUGGUGCAAAGAAACAUGGACUUGAAGUUCAACAUCAA
GAGCAGUAGCAGUUCCCCUGACUCUCGGGCAGUGACAUGUGGAAUGGCGUCUCUGUCUGC
AGAGAAGGUCACACUGGACCAAAGGGACUAUGAGAAGUAUUCAGUGUCCUGCCAGGAGGA
UGUCACCUGCCCAACUGCCGAGGAGACCCUGCCCAUUGAACUGGCGUUGGAAGCACGGCA
GCAGAAUAAAUAUGAGAACUACAGCACCAGCUUCUUCAUCAGGGACAUCAUCAAACCAGA
CCCGCCCAAGAACUUGCAGAUGAAGCCUUUGAAGAACUCACAGGUGGAGGUCAGCUGGGA
GUACCCUGACUCCUGGAGCACUCCCCAUUCCUACUUCUCCCUCAAGUUCUUUGUUCGAAU
CCAGCGCAAGAAAGAAAAGAUGAAGGAGACAGAGGAGGGGUGUAACCAGAAAGGUGCGUU
CCUCGUAGAGAAGACAUCUACCGAAGUCCAAUGCAAAGGCGGGAAUGUCUGCGUGCAAGC
UCAGGAUCGCUAUUACAAUUCCUCAUGCAGCAAGUGGGCAUGUGUUCCCUGCAGAGUCCG
AUCGGUUCCUGGAGUAGGGGUACCUGGAGUGGGCAGGGUCAUACCGGUCUCUGGACCUGC
CAGGUGUCUUAGCCAGUCCCGAAACCUGCUGAAGACCACAGAUGACAUGGUGAAGACGGC
CAGAGAAAAGCUGAAACAUUAUUCCUGCACUGCUGAAGACAUCGAUCAUGAAGACAUCAC
ACGGGACCAAACCAGCACAUUGAAGACCUGUUUACCACUGGAACUACACAAGAACGAGAG
UUGCCUGGCUACUAGAGAGACUUCUUCCACAACAAGAGGGAGCUGCCUGCCCCCACAGAA
GACGUCUUUGAUGAUGACCCUGUGCCUUGGUAGCAUCUAUGAGGACUUGAAGAUGUACCA
GACAGAGUUCCAGGCCAUCAACGCAGCACUUCAGAAUCACAACCAUCAGCAGAUCAUUCU
AGACAAGGGCAUGCUGGUGGCCAUCGAUGAGCUGAUGCAGUCUCUGAAUCAUAAUGGCGA
GACUCUGCGCCAGAAACCUCCUGUGGGAGAAGCAGACCCUUACAGAGUGAAAAUGAAGCU
CUGCAUCCUGCUUCACGCCUUCAGCACCCGCGUCGUGACCAUCAACAGGGUGAUGGGCUA
UCUGAGCUCCGCCUAAAGugauaaccgcggugucaaaaaccgcguggacgugguuaacau
cccugcugggaggaucagccguaauuauuauaauuggcuuggugcuggcuacuauugugg
ccauguacgugcugaccaaccagaaacauaauugaauacagcagcaauuggcaagcugcu
uacauagaacucgcggcgauuggcaugccgccuuaaaauuuuuauuuuauuuuuuucuuu
ucuuuuccgaaucggauuuuguuuuaauauuucaaaaaaaaaaaaaaaaaaaaaaaaaaa
aaaaaaaaaaaaaaaaaaa
SEQ ID NO: 33: DNA Sequence Encoding SEAP
tcgcgcgtttcggtgatgacggtgaaaacctctgacacatgcagctcccggagacggtca
cagcttgtctgtaagcggatgccgggagcagacaagcccgtcagggcgcgtcagcgggtg
ttggcgggtgtcggggctggcttaactatgcggcatcagagcagattgtactgagagtgc
accatatgcggtgtgaaataccgcacagatgcgtaaggagaaaataccgcatcagattgg
ctattggccattgcatacgttgtatccatatcataatatgtacatttatattggctcatg
tccaacattaccgccatgttgacattgattattgactagttattaatagtaatcaattac
ggggtcattagttcatagcccatatatggagttccgcgttacataacttacggtaaatgg
cccgcctggctgaccgcccaacgacccccgcccattgacgtcaataatgacgtatgttcc
catagtaacgccaatagggactttccattgacgtcaatgggtggagtatttacggtaaac
tgcccacttggcagtacatcaagtgtatcatatgccaagtacgccccctattgacgtcaa
tgacggtaaatggcccgcctggcattatgcccagtacatgaccttatgggactttcctac
ttggcagtacatctacgtattagtcatcgctattaccatggtgatgcggttttggcagta
catcaatgggcgtggatagcggtttgactcacggggatttccaagtctccaccccattga
cgtcaatgggagtttgttttggcaccaaaatcaacgggactttccaaaatgtcgtaacaa
ctccgccccattgacgcaaatgggcggtaggcgtgtacggtgggaggtctatataagcag
agctcgtttagtgaaccgtcagatcgcctggagacgccatccacgctgttttgacctcca
tagaagacaccgggaccgatccagcctccgcggccgggaacggtgcattggaacgcggat
tccccgtgccaagagtgacgtaagtaccgcctatagactctataggcacacccctttggc
tcttatgcatgctatactgtttttggcttggggcctatacacccccgcttccttatgcta
taggtgatggtatagcttagcctataggtgtgggttattgaccattattgaccactcccc
tattggtgacgatactttccattactaatccataacatggctctttgccacaactatctc
tattggctatatgccaatactctgtccttcagagactgacacggactctgtatttttaca
ggatggggtcccatttattatttacaaattcacatatacaacaacgccgtcccccgtgcc
cgcagtttttattaaacatagcgtgggatctccacgcgaatctcgggtacgtgttccgga
catgggctcttctccggtagcggcggagcttccacatccgagccctggtcccatgcctcc
agcggctcatggtcgctcggcagctccttgctcctaacagtggaggccagacttaggcac
agcacaatgcccaccaccaccagtgtgccgcacaaggccgtggcggtagggtatgtgtct
gaaaatgagcgtggagattgggctcgcacggctgacgcagatggaagacttaaggcagcg
gcagaagaagatgcaggcagctgagttgttgtattctgataagagtcagaggtaactccc
gttgcggtgctgttaacggtggagggcagtgtagtctgagcagtactcgttgctgccgcg
cgcgccaccagacataatagctgacagactaacagactgttcctttccatgggtcttttc
tgcagtcaccgtcgtcgacaagcttcctgcatgctgctgctgctgctgctgctgggcctg
aggctacagctctccctgggcatcatcccagttgaggaggagaacccggacttctggaac
cgcgaggcagccgaggccctgggtgccgccaagaagctgcagcctgcacagacagccgcc
aagaacctcatcatcttcctgggcgatgggatgggggtgtctacggtgacagctgccagg
atcctaaaagggcagaagaaggacaaactggggcctgagatacccctggccatggaccgc
ttcccatatgtggctctgtccaagacatacaatgtagacaaacatgtgccagacagtgga
gccacagccacggcctacctgtgcggggtcaagggcaacttccagaccattggcttgagt
gcagccgcccgctttaaccagtgcaacacgacacgcggcaacgaggtcatctccgtgatg
aatcgggccaagaaagcagggaagtcagtgggagtggtaaccaccacacgagtgcagcac
gcctcgccagccggcacctacgcccacacggtgaaccgcaactggtactcggacgccgac
gtgcctgcctcggcccgccaggaggggtgccaggacatcgctacgcagctcatctccaac
atggacattgacgtgatcctaggtggaggccgaaagtacatgtttcgcatgggaacccca
gaccctgagtacccagatgactacagccaaggtgggaccaggctggacgggaagaatctg
gtgcaggaatggctggcgaagcgccagggtgcccggtatgtgtggaaccgcactgagctc
atgcaggcttccctggacccgtctgtgacccatctcatgggtctctttgagcctggagac
atgaaatacgagatccaccgagactccacactggacccctccctgatggagatgacagag
gctgccctgcgcctgctgagcaggaacccccgcggcttcttcctcttcgtggagggtggt
cgcatcgaccatggtcatcatgaaagcagggcttaccgggcactgactgagacgatcatg
ttcgacgacgccattgagagggcgggccagctcaccagcgaggaggacacgctgagcctc
gtcactgccgaccactcccacgtcttctccttcggaggctaccccctgcgagggagctcc
atcttcgggctggcccctggcaaggcccgggacaggaaggcctacacggtcctcctatac
ggaaacggtccaggctatgtgctcaaggacggcgcccggccggatgttaccgagagcgag
agcgggagccccgagtatcggcagcagtcagcagtgcccctggacgaagagacccacgca
ggcgaggacgtggcggtgttcgcgcgcggcccgcaggcgcacctggttcacggcgtgcag
gagcagaccttcatagcgcacgtcatggccttcgccgcctgcctggagccctacaccgcc
tgcgacctggcgccccccgccggcaccaccgacgccgcgcacccgggttaacccgtggtc
agatccagatccagatcacttctggctaataaaagatcagagctctagagatctgtgtgt
tggttttttgtggatctgctgtgccttctagttgccagccatctgttgtttgcccctccc
ccgtgccttccttgaccctggaaggtgccactcccactgtcctttcctaataaaatgagg
aaattgcatcgcattgtctgagtaggtgtcattctattctggggggtggggtggggcagc
acagcaagggggaggattgggaagacaatagcaggcatgctggggatgcggtgggctcta
tgggtacctctctctctctctctctctctctctctctctctctctctcggtacctctctc
tctctctctctctctctctctctctctctctctcggtaccaggtgctgaagaattgaccc
ggttcctcctgggccagaaagaagcaggcacatccccttctctgtgacacaccctgtcca
cgcccctggttcttagttccagccccactcataggacactcatagctcaggagggctccg
ccttcaatcccacccgctaaagtacttggagcggtctctccctccctcatcagcccacca
aaccaaacctagcctccaagagtgggaagaaattaaagcaagataggctattaagtgcag
agggagagaaaatgcctccaacatgtgaggaagtaatgagagaaatcatagaatttcttc
cgcttcctcgctcactgactcgctgcgctcggtcgttcggctgcggcgagcggtatcagc
tcactcaaaggcggtaatacggttatccacagaatcaggggataacgcaggaaagaacat
gtgagcaaaaggccagcaaaaggccaggaaccgtaaaaaggccgcgttgctggcgttttt
ccataggctccgcccccctgacgagcatcacaaaaatcgacgctcaagtcagaggtggcg
aaacccgacaggactataaagataccaggcgtttccccctggaagctccctcgtgcgctc
tcctgttccgaccctgccgcttaccggatacctgtccgcctttctcccttcgggaagcgt
ggcgctttctcaatgctcacgctgtaggtatctcagttcggtgtaggtcgttcgctccaa
gctgggctgtgtgcacgaaccccccgttcagcccgaccgctgcgccttatccggtaacta
tcgtcttgagtccaacccggtaagacacgacttatcgccactggcagcagccactggtaa
caggattagcagagcgaggtatgtaggcggtgctacagagttcttgaagtggtggcctaa
ctacggctacactagaaggacagtatttggtatctgcgctctgctgaagccagttacctt
cggaaaaagagttggtagctcttgatccggcaaacaaaccaccgctggtagcggtggttt
ttttgtttgcaagcagcagattacgcgcagaaaaaaaggatctcaagaagatcctttgat
cttttctacggggtctgacgctcagtggaacgaaaactcacgttaagggattttggtcat
gagattatcaaaaaggatcttcacctagatccttttaaattaaaaatgaagttttaaatc
aatctaaagtatatatgagtaaacttggtctgacagttaccaatgcttaatcagtgaggc
acctatctcagcgatctgtctatttcgttcatccatagttgcctgactccgggggggggg
ggcgctgaggtctgcctcgtgaagaaggtgttgctgactcataccaggcctgaatcgccc
catcatccagccagaaagtgagggagccacggttgatgagagctttgttgtaggtggacc
agttggtgattttgaacttttgctttgccacggaacggtctgcgttgtcgggaagatgcg
tgatctgatccttcaactcagcaaaagttcgatttattcaacaaagccgccgtcccgtca
agtcagcgtaatgctctgccagtgttacaaccaattaaccaattctgattagaaaaactc
atcgagcatcaaatgaaactgcaatttattcatatcaggattatcaataccatatttttg
aaaaagccgtttctgtaatgaaggagaaaactcaccgaggcagttccataggatggcaag
atcctggtatcggtctgcgattccgactcgtccaacatcaatacaacctattaatttccc
ctcgtcaaaaataaggttatcaagtgagaaatcaccatgagtgacgactgaatccggtga
gaatggcaaaagcttatgcatttctttccagacttgttcaacaggccagccattacgctc
gtcatcaaaatcactcgcatcaaccaaaccgttattcattcgtgattgcgcctgagcgag
acgaaatacgcgatcgctgttaaaaggacaattacaaacaggaatcgaatgcaaccggcg
caggaacactgccagcgcatcaacaatattttcacctgaatcaggatattcttctaatac
ctggaatgctgttttcccggggatcgcagtggtgagtaaccatgcatcatcaggagtacg
gataaaatgcttgatggtcggaagaggcataaattccgtcagccagtttagtctgaccat
ctcatctgtaacatcattggcaacgctacctttgccatgtttcagaaacaactctggcgc
atcgggcttcccatacaatcgatagattgtcgcacctgattgcccgacattatcgcgagc
ccatttatacccatataaatcagcatccatgttggaatttaatcgcggcctcgagcaaga
cgtttcccgttgaatatggctcataacaccccttgtattactgtttatgtaagcagacag
ttttattgttcatgatgatatatttttatcttgtgcaatgtaacatcagagattttgaga
cacaacgtggctttccccccccccccattattgaagcatttatcagggttattgtctcat
gagcggatacatatttgaatgtatttagaaaaataaacaaataggggttccgcgcacatt
tccccgaaaagtgccacctgacgtctaagaaaccattattatcatgacattaacctataa
aaataggcgtatcacgaggccctttcgtc
SEQ ID NO: 34: Vector Backbone RNA Sequence 1-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaauaacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaauaagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuuaguuggacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaaauuaucuugcauccacccucugcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucauuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaciggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacauaauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acauucauacacuguuugauaugucggcugaagacuuugacgcuauuauagccgagcacu
uccagccuggggauuguguucuggaaacugacaucgcgucguuugauaaaagugaggacg
acgccauggcucugaccgcguuaaugauucuggaagacuuagguguggacgcagagcugu
ugacgcugauugaggcggcuuucggcgaaauuucaucaauacauuugcccacuaaaacua
aauuuaaauucggagccaugaugaaaucuggaauguuccucacacuguuugugaacacag
ucauuaacauuguaaucgcaagcagaguguugagagaacggcuaaccggaucaccaugug
cagcauucauuggagaugacaauaucgugaaaggagucaaaucggacaaauuaauggcag
acaggugcgccaccugguugaauauggaagucaagauuauagaugcuguggugggcgaga
aagcgccuuauuucuguggaggguuuauuuugugugacuccgugaccggcacagcgugcc
guguggcagacccccuaaaaaggcuguuuaagcuuggcaaaccucuggcagcagacgaug
aacaugaugaugacaggagaagggcauugcaugaagagucaacacgcuggaaccgagugg
guauucuuucagagcugugcaaggcaguagaaucaagguaugaaaccguaggaacuucca
ucauaguuauggccaugacuacucuagcuagcaguguuaaaucauucagcuaccugagag
gggccccuauaacucucuacggcuaaccugaauggacuacgacauagucuaguccgccaa
gXugauaaccgcggugucaaaaaccgcguggacgugguuaacaucccugcugggaggauc
agccguaauuauuauaauuggcuuggugcuggcuacuauuguggccauguacgugcugac
caaccagaaacauaauugaauacagcagcaauuggcaagcugcuuacauagaacucgcgg
cgauuggcaugccgccuuaaaauuuuuauuuuauuuuuucuuuucuuuuccgaaucggau
uuuguuuuuaauauuucaaaaaaaaaaaaaaaaaaaaaaaaaaaaaaaaaaaaaaaaaaa
aa
SEQ ID NO: 35: Vector Backbone RNA Sequence 2-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaauaacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaauaagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucauuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaccggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacauaauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acauucauacacuguuugauaugucggcugaagacuuugacgcuauuauagccgagcacu
uccagccuggggauuguguucuggaaacugacaucgcgucguuugauaaaagugaggacg
acgccauggcucugaccgcguuaaugauucuggaagacuuagguguggacgcagagcugu
ugacgcugauugaggcggcuuucggcgaaauuucaucaauacauuugcccacuaaaacua
aauuuaaauucggagccaugaugaaaucuggaauguuccucacacuguuugugaacacag
ucauuaacauuguaaucgcaagcagaguguugagagaacggcuaaccggaucaccaugug
cagcauucauuggagaugacaauaucgugaaaggagucaaaucggacaaauuaauggcag
acaggugcgccaccugguugaauauggaagucaagauuauagaugcuguggugggcgaga
aagcgccuuauuucuguggaggguuuauuuugugugacuccgugaccggcacagcgugcc
guguggcagacccccuaaaaaggcuguuuaagcuuggcaaaccucuggcagcagacgaug
aacaugaugaugacaggagaagggcauugcaugaagagucaacacgcuggaccaugacua
cucuagcuagcaguguuaaaucauucagcuaccugagaggggccccuauaacucucuacg
gcuaaccugaauggacuacgacauagucuaguccgccaagXugauaaccgcggugucaaa
aaccgcguggacgugguuaacaucccugcugggaggaucagccguaauuauuauaauugg
cuuggugcuggcuacuauuguggccauguacgugcugaccaaccagaaacauaauugaau
acagcagcaauuggcaagcugcuuacauagaacucgcggcgauuggcaugccgccuuaaa
auuuuuauuuuauuuuuucuuuucuuuuccgaaucggauuuuguuuuuaauauuucaaaa
aaaaaaaaaaaaaaaaaaaaaaaaaaaaaaaaaaaaaaaaa
SEQ ID NO: 36: Vector Backbone RNA Sequence 3-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaauaacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaauaagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucauuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaccggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacauaauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acaaaacugacaucgcgucguuugauaaaagugaggacgacgccauggcucugaccgcgu
uaaugauucuggaagacuuagguguggacgcagagcuguugacgcugauugaggcggcuu
ucggcgaaauuucaucaauacauuugcccacuaaaacuaaauuuaaauucggagccauga
ugaaaucuggaauguuccucacacuguuugugaacacagucauuaacauuguaaucgcaa
gcagaguguugagagaacggcuaaccggaucaccaugugcagcauucauuggagaugaca
auaucgugaaaggagucaaaucggacaaauuaauggcagacaggugcgccaccugguuga
auauggaagucaagauuauagaugcuguggugggcgagaaagcgccuuauuucuguggag
gguuuauuuugugugacuccgugaccggcacagcgugccguguggcagacccccuaaaaa
ggcuguuuaagcuuggcaaaccucuggcagcagacgaugaacaugaugaugacaggagaa
gggcauugcaugaagagucaacacgcuggaaccgaguggguauucuuucagagcugugca
aggcaguagaaucaagguaugaaaccguaggaacuuccaucauaguuauggccaugacua
cucuagcuagcaguguuaaaucauucagcuaccugagaggggccccuauaacucucuacg
gcuaaccugaauggacuacgacauagucuaguccgccaagXuaguaaccgcggugucaaa
aaccgcguggacgugguuaacaucccugcugggaggaucagccguaauuauuauaauugg
cuuggugcuggcuacuauuguggccauguacgugcugaccaaccagaaacauaauugaau
acagcagcaauuggcaagcugcuuacauagaacucgcggcgauuggcaugccgccuuaaa
auuuuuauuuuauuuuuucuuuucuuuuccgaaucggauuuuguuuuuaauauuucaaaa
aaaaaaaaaaaaaaaaaaaaaaaaaaaaaaaaaaaaaaaaa
SEQ ID NO: 37: Vector Backbone RNA Sequence 4-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaauaacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaauaagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucauuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaciggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguuctuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacauaauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acauucauacacuguuugauaugucggcugaagacuuugacgcuauuauagccgagcacu
uccagccuggggauuguguucuggaaacugacaucgcgucguuugauaaaagugaggacg
acgccauggcucugaccgcguuaaugauucuggaagacuuagguguggacgcagagcugu
ugacgcugauugaggcggcuuucggcgaaauuucaucaauacauuugcccacuaaaacua
aauuuaaauucggagccaugaugaaaucuggaauguuccucacacuguuugugaacacag
ucauuaacauuguaaucgcaagcagaguguugagagaacggcuaaccggaucaccaugug
cagcauucauuggagaugacaauaucgugaaaggagucaaaucggacaaauuaauggcag
acaggugcgccaccugguugaauauggaagucaagauuauagaugcuguggugggcgaga
aagcgccuuagcuuggcaaaccucuggcagcagacgaugaacaugaugaugacaggagaa
gggcauugcaugaagagucaacacgcuggaaccgaguggguauucuuucagagcugugca
aggcaguagaaucaagguaugaaaccguaggaacuuccaucauaguuauggccaugacua
cucuagcuagcaguguuaaaucauucagcuaccugagaggggccccuauaacucucuacg
gcuaaccugaauggacuacgacauagucuaguccgccaagXugauaaccgcggugucaaa
aaccgcguggacgugguuaacaucccugcugggaggaucagccguaauuauuauaauugg
cuuggugcuggcuacuauuguggccauguacgugcugaccaaccagaaacauaauugaau
acagcagcaauuggcaagcugcuuacauagaacucgcggcgauuggcaugccgccuuaaa
auuuuuauuuuauuuuuucuuuucuuuuccgaaucggauuuuguuuuuaauauuucaaaa
aaaaaaaaaaaaaaaaaaaaaaaaaaaaaaaaaaaaaaaaa
SEQ ID NO: 38: Vector Backbone RNA Sequence 5-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaataacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaataagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucatuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuataaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaccggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguatauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacauaauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acauucauacacuguuugauaugucggcugaagacuuugacgcuauuauagccgagcacu
uccagccuggggauuguguucuggaaacugacaucgcgucguuugauaaaagugaggacg
acgccauggcucugaccgcguuaaugauucuggaagacuuagguguggacgcagagcugu
ugacgcugauugaggcggcuuucggcgaaauuucaucaauacauuugcccacuaaaacua
aauuuaaauucggagccaugaugaaaucuggaauguuccucacacuguuugugaacacag
ucauuaacauuguaaucgcaagcagaguguugagagaacggcuaaccggaucaccaugug
cagcauucauuggagaugacaauaucgugaaaggagucaaaucggacaauuucuguggag
gguuuauuuugugugacuccgugaccggcacagcgugccguguggcagacccccuaaaaa
ggcuguuuaagcuuggcaaaccucuggcagcagacgaugaacaugaugaugacaggagaa
gggcauugcaugaagagucaacacgcuggaaccgaguggguauucuuucagagcugugca
aggcaguagaaucaagguaugaaaccguaggaacuuccaucauaguuauggccaugacua
cucuagcuagcaguguuaaaucauucagcuaccugagaggggccccuauaacucucuacg
gcuaaccugaauggacuacgacauagucuaguccgccaagXugauaaccgcggugucaaa
aaccgcguggacgugguuaacaucccugcugggaggaucagccguaauuauuauaauugg
cuuggugcuggcuacuauuguggccauguacgugcugaccaaccagaaacauaauugaau
acagcagcaauuggcaagcugcuuacauagaacucgcggcgauuggcaugccgccuuaaa
auuuuuauuuuauuuuuucuuuucuuuuccgaaucggauuuuguuuuuaauauuucaaaa
aaaaaaaaaaaaaaaaaaaaaaaaaaaaaaaaaaaaaaaaa
SEQ ID NO: 39: Vector Backbone RNA Sequence 6-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguaucuguccgaugagau
gugcggaagauccggacagauuguauaaguaugcaacuaagcugaagaaaaacuguaagg
aaataacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaataagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcuuuagugccggugcugaagaccgcuggca
uagacaugaccacugaacaauggaacacuguggauuauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcuuagcauguugaccaagaaagcuugucugcauc
ugaaucccggcggaaccugugucagcauagguuaugguuacgcugacagggccagcgaaa
gcaucatuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaccggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaauuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucuuacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugaacccacaauacgaucggcagugc
cuucagcgauccagaacacgcuccagaacguccuggcagcugccacaaaaagaaauugca
augucacgcaaaugagagaauugcccguauuggauucggcggccuuuaauguggaaugcu
ucaagaaauaugcguguaauaaugaauauugggaaacguuuaaagaaaaccccaucaggc
uuacugaagaaaacgugguaaauuacauuaccaaauuaaaaggaccaaaagcugcugcuc
uuuuugcgaagacacauaauuugaauauguugcaggacauaccaauggacagguuuguaa
uggacuuaaagagagacgugaaagugacuccaggaacaaaacauacugaagaacggccca
agguacaggugauccaggcugccgauccgcuagcaacagcguaucugugcggaauccacc
gagagcugguuaggagauuaaaugcgguccugcuuccgaacauucauacacuguuugaua
ugucggcugaagacuuugacgcuauuauagccgagcacuuccagccuggggauuguguuc
uggaaacugacaucgcgucguuugauaaaagugaggacgacgccauggcucugaccgcgu
uaaugauucuggaagacuuagguguggacgcagagcuguugacgcugauugaggcggcuu
ucggcgaaauuucaucaauacauuugcccacuaaaacuaaauuuaaauucggagccauga
ugaaaucuggaauguuccucacacuguuugugaacacagucauuaacauuguaaucgcaa
gcagaguguugagagaacggcuaaccggaucaccaugugcagcauucauuggagaugaca
auaucgugaaaggagucaaaucggacaaauuaauggcagacaggugcgccaccugguuga
auauggaagucaagauuauagaugcuguggugggcgagaaagcgccuuauuucuguggag
gguuuauuuugugugacuccgugaccggcacagcgugccguguggcagacccccuaaaaa
ggcuguuuaagcuuggcaaaccucuggcagcagacgaugaacaugaugaugacaggagaa
gggcauugcaugaagagucaacacgcuggaaccgaguggguauucuuucagagcugugca
aggcaguagaaucaagguaugaaaccguaggaacuuccaucauaguuauggccaugacua
cucuagcuagcaguguuaaaucauucagcuaccugagaggggccccuauaacucucuacg
gcuaaccugaauggacuacgacauagucuaguccgccaagAUGUUUCUGCUCACAACCAA
ACGCACUAUGUUUGUUUUCCUCGUGCUGCUCCCUUUGGUAAGUUCUCAGUGUGUAAACCU
GAGAACACGAACCCAGUUGCCUCCAGCUUAUACCAACUCAUUUACUCGCGGAGUAUAUUA
UCCCGAUAAGGUCUUUAGAAGUAGCGUGUUGCACUCUACACAGGAUCUGUUCUUGCCCUU
CUUUAGUAACGUUACCUGGUUUCAUGCAAUACAUGUGAGCGGAACAAAUGGAACAAAAAG
AUUUGACAAUCCAGUGCUUCCAUUUAAUGAUGGGGUUUACUUUGCCAGUAUCGAAAAGUC
AAACAUAAUCCGGGGGUGGAUCUUUGGAACCACUUUGGACUCUAAGACACAGUCUCUCCU
CAUAGUAAACAACGCCACCAAUGUUGUCAUAAAAGUAUGCGAAUUUCAGUUUUGCAACGA
UCCCUUUCUCGACGUGUAUUACCAUAAGAAUAAUAAAUCCUGGAUGGAGUCUGGGGUUUA
UAGUAGUGCUAAUAAUUGCACUUUCGAAUACGUGUCCCAACCAUUCCUCAUGGACCUUGA
GGGCAAACAGGGGAAUUUUAAAAACUUGCGCGAAUUUGUCUUUAAGAAUAUCGACGGAUA
CUUUAAGAUCUAUAGUAAACACACUCCUAUCAACCUCGUUCGGGAUCUUCCCCAAGGCUU
UUCUGCUCUCGAACCCCUCGUAGACUUGCCAAUUGGGAUAAAUAUCACUCGCUUUCAAAC
UUUGCUUGCCCUCCACAGGAGCUACCUGACACCCGGCGACUCUUCUUCUGGUUUGACCGC
CGGCGCCGCUGCCUAUUAUGUUGGUUACCUUCAGCCACGAACAUUCUUGCUCAAGUAUAA
CGAGAAUGGCACCAUUACCGACGCCGUCGAUUGUGCAUUGGAUCCCUUGUCUGAAACAAA
AUGUACCUUGAAGUCCUUUACCGUAGAGAAAGGCAUAUACCAGACUUCCAACUUCCGAGU
UCAGCCUACAGAAUCCAUCGUACGAUUUCCCAACAUCACAAACCUCUGCCCUUUCGGUGA
AGUAUUUAAUGCUACACGCUUCGCUUCAGUCUAUGCCUGGAAUAGGAAGCGCAUAUCAAA
UUGCGUGGCCGAUUAUUCAGUCCUCUAUAAUAGCGCAUCCUUCAGUACUUUCAAGUGCUA
CGGCGUUUCCCCCACCAAACUCAAUGAUCUUUGCUUCACCAACGUCUAUGCUGACAGUUU
UGUCAUACGAGGCGACGAAGUACGCCAGAUUGCCCCCGGGCAGACAGGUAAUAUUGCUGA
UUAUAAUUAUAAACUCCCAGAUGACUUUACUGGAUGCGUCAUAGCCUGGAAUUCCAACAA
UCUAGAUUCCAAGGUUGGUGGGAAUUAUAAUUACCGUUAUCGACUGUUCAGAAAGAGUAA
CUUGAAACCAUUUGAGAGAGACAUAUCCACCGAGAUUUACCAGGCAGGCAGUAAGCCUUG
UAACGGCGUUGAGGGAUUUAACUGCUAUUUUCCUUUGCAAUCCUAUGGCUUUCAACCAAC
AAACGGGGUUGGCUAUCAACCCUAUCGAGUGGUUGUCCUCAGCUUUGAACUUUUGCACGC
UCCCGCCACAGUCUGCGGACCAAAAAAGAGUACAAAUCUUGUCAAGAAUAAGUGCGUAAA
UUUCAAUUUCAAUGGCCUUACAGGAACAGGCGUGCUGACUGAGUCAAACAAGAAUUUCCU
GCCAUUUCAGCAGUUUGGGCGGGAUAUAGCAGACACAACUGACGCUGUACGCGAUCCUCA
GACUUUGGAGAUCUUGGACAUCACUCCCUGUUCUUUCGGAGGGGUAUCUGUCAUCACCCC
CGGAACUAAUACAUCAAAUCAGGUCGCUGUGUUGUACCAAGGUGUCAACUGCACAGAAGU
CCCCGUUGCUAUACACGCAGACCAGCUCACCCCCACAUGGCGGGUGUACUCAACUGGCUC
AAACGUAUUCCAGACCAGAGCUGGGUGCUUGAUCGGUGCUGAACACGUGAACAAUAGCUA
UGAAUGCGAUAUUCCCAUCGGUGCCGGGAUCUGCGCUAGCUAUCAGACACAGACCAAUUC
CCGCAGGCGGGCUCGCUCUGUAGCAUCCCAGUCUAUUAUUGCCUACACUAUGUCAUUGGG
CGCCGAGAAUAGCGUCGCAUAUUCAAAUAAUUCUAUUGCAAUACCCACCAACUUCACAAU
CUCCGUAACUACAGAAAUACUUCCAGUUUCCAUGACAAAGACAUCAGUGGAUUGUACAAU
GUAUAUAUGCGGAGAUUCCACAGAAUGUUCAAAUUUGCUCUUGCAGUACGGCUCCUUCUG
CACCCAGCUCAACAGGGCACUUACAGGUAUUGCUGUCGAACAGGACAAGAACACACAAGA
AGUCUUCGCCCAAGUCAAACAGAUAUACAAAACUCCUCCCAUAAAGGAUUUUGGCGGCUU
CAACUUUAGUCAGAUCCUCCCAGACCCUUCAAAACCAUCUAAACGAUCAUUUAUUGAAGA
UCUGCUGUUCAACAAGGUCACUCUUGCCGAUGCUGGAUUCAUUAAGCAAUACGGUGACUG
CCUUGGUGAUAUUGCUGCCCGAGAUCUGAUCUGUGCCCAGAAAUUCAACGGGCUCACUGU
ACUCCCUCCACUGCUCACAGACGAAAUGAUUGCACAGUACACAAGUGCCCUGUUGGCAGG
CACAAUCACUAGCGGCUGGACCUUUGGCGCAGGUGCAGCACUCCAAAUACCUUUUGCCAU
GCAGAUGGCCUAUCGGUUUAAUGGGAUAGGCGUGACUCAAAAUGUCCUCUACGAAAACCA
AAAGUUGAUAGCUAACCAAUUCAAUUCAGCAAUCGGGAAGAUACAGGAUUCACUGUCUAG
UACUGCUAGUGCCCUUGGUAAGCUGCAGAACGUUGUCAACCAGAAUGCUCAAGCUCUGAA
UACAUUGGUUAAGCAGCUCUCUAGUAAUUUUGGGGCCAUCUCUUCAGUACUUAAUGAUAU
UUUGAGCCGAUUGGACCCACCUGAAGCUGAAGUACAGAUCGACAGGCUGAUAACAGGCCG
GCUCCAAUCCCUCCAAACAUACGUGACACAACAACUCAUACGCGCAGCCGAAAUCCGAGC
CAGCGCUAACCUGGCAGCUACCAAGAUGUCAGAAUGCGUUCUGGGCCAGAGUAAACGCGU
AGAUUUCUGCGGGAAAGGGUACCACCUGAUGUCCUUUCCACAAUCUGCACCUCACGGGGU
CGUCUUUUUGCAUGUAACAUACGUACCCGCACAAGAGAAGAAUUUUACUACCGCUCCUGC
CAUCUGUCAUGACGGGAAAGCUCAUUUUCCUCGCGAAGGUGUGUUUGUAUCUAAUGGUAC
ACAUUGGUUUGUCACACAGCGGAAUUUCUAUGAACCCCAGAUCAUUACAACUGACAACAC
UUUUGUUUCCGGGAAUUGUGACGUGGUCAUAGGAAUCGUAAAUAACACUGUAUAUGAUCC
CCUCCAACCAGAGCUGGACUCUUUUAAAGAAGAACUGGAUAAAUAUUUCAAGAACCACAC
AAGUCCCGACGUGGACCUUGGGGACAUAAGUGGUAUUAACGCAUCUGUGGUUAACAUUCA
AAAGGAAAUCGACAGACUCAACGAGGUGGCCAAAAACCUGAACGAAAGCUUGAUAGAUCU
CCAGGAGUUGGGCAAGUAUGAACAGUACAUUAAAUGGCCAUGGUACAUAUGGCUUGGCUU
UAUCGCUGGCCUUAUCGCCAUCGUAAUGGUUACAAUCAUGCUGUGCUGCAUGACCUCCUG
CUGUUCUUGUUUGAAAGGGUGUUGUUCUUGUGGUAGUUGUUGCAAGUUUGACGAAGAUGA
UUCCGAACCUGUUCUUAAGGGGGUAAAGCUUCACUAUACAUGAuaaccgcggugucaaaa
accgcguggacgugguuaacaucccugcugggaggaucagccguaauuauuauaauuggc
uuggugcuggcuacuauuguggccauguacgugcugaccaaccagaaacauaauugaaua
cagcagcaauuggcaagcugcuuacauagaacucgcggcgauuggcaugccgccuuaaaa
uuuuuauuuuauuuuuucuuuucuuuuccgaaucggauuuuguuuuaauauuucaaaaaa
aaaaaaaaaaaaaaaaaaaaaaaaaaaaaaaaaaaaaaaaaaaa
SEQ ID NO: 40: Vector Backbone RNA Sequence 7-
X is any nucleotide: A, G, U, C.
auaggcggcgcaugagagaagcccagaccaauuaccuacccaaaauggagaaaguucacg
uugacaucgaggaagacagcccauuccucagagcuuugcagcggagcuucccgcaguuug
agguagaagccaagcaggucacugauaaugaccaugcuaaugccagagcguuuucgcauc
uggcuucaaaacugaucgaaacggagguggacccauccgacacgauccuugacauuggaa
gugcgcccgcccgcagaauguauucuaagcacaaguaucauuguatcuguccgaugagau
gugcggaagauccggacagauuguauaaguaugcaacuaagcugaagaaaaacuguaagg
aaauaacugauaaggaauuggacaagaaaaugaaggagcuggccgccgucaugagcgacc
cugaccuggaaacugagacuaugugccuccacgacgacgagucgugucgcuacgaagggc
aagucgcuguuuaccaggauguauacgcgguugacggaccgacaagucucuaucaccaag
ccaauaagggaguuagagucgccuacuggauaggcuuugacaccaccccuuuuauguuua
agaacuuggcuggagcauauccaucauacucuaccaacugggccgacgaaaccguguuaa
cggcucguaacauaggccuaugcagcucugacguuauggagcggucacguagagggaugu
ccauucuuagaaagaaguauuugaaaccauccaacaauguucuauucucuguuggcucga
ccaucuaccacgagaagagggacuuacugaggagcuggcaccugccgucuguauuucacu
uacguggcaagcaaaauuacacaugucggugugagacuauaguuaguugcgacggguacg
ucguuaaaagaauagcuaucaguccaggccuguaugggaagccuucaggcuaugcugcua
cgaugcaccgcgagggauucuugugcugcaaagugacagacacauugaacggggagaggg
ucucuuuucccgugugcacguaugugccagcuacauugugugaccaaaugacuggcauac
uggcaacagaugucagugcggacgacgcgcaaaaacugcugguugggcucaaccagcgua
uagucgucaacggucgcacccagagaaacaccaauaccaugaaaaauuaccuuuugcccg
uaguggcccaggcauuugcuaggugggcaaaggaauauaaggaagaucaagaagaugaaa
ggccacuaggacuacgagauagacaguuagucaugggguguuguugggcuuuuagaaggc
acaagauaacaucuauuuauaagcgcccggauacccaaaccaucaucaaagugaacagcg
auuuccacucauucgugcugcccaggauaggcaguaacacauuggagaucgggcugagaa
caagaaucaggaaaauguuagaggagcacaaggagccgucaccucucauuaccgccgagg
acguacaagaagcuaagugcgcagccgaugaggcuaaggaggugcgugaagccgaggagu
ugcgcgcagcucuaccaccuuuggcagcugauguugaggagcccacucuggaggcagacg
ucgacuugauguuacaagaggcuggggccggcucaguggagacaccucguggcuugauaa
agguuaccagcuacgauggcgaggacaagaucggcucuuacgcugugcuuucuccgcagg
cuguacucaagagugaaaaauuaucuugcauccacccucucgcugaacaagucauaguga
uaacacacucuggccgaaaagggcguuaugccguggaaccauaccaugguaaaguagugg
ugccagagggacaugcaauacccguccaggacuuucaagcucugagugaaagugccacca
uuguguacaacgaacgugaguucguaaacagguaccugcaccauauugccacacauggag
gagcgcugaacacugaugaagaauauuacaaaacugucaagcccagcgagcacgacggcg
aauaccuguacgacaucgacaggaaacagugcgucaagaaagaacuagucacugggcuag
ggcucacaggcgagcugguggauccucccuuccaugaauucgccuacgagagucugagaa
cacgaccagccgcuccuuaccaaguaccaaccauagggguguauggcgugccaggaucag
gcaagucuggcaucauuaaaagcgcagucaccaaaaaagaucuaguggugagcgccaaga
aagaaaacugugcagaaauuauaagggacgucaagaaaaugaaagggcuggacgucaaug
ccagaacuguggacucagugcucuugaauggaugcaaacaccccguagagacccuguaua
uugacgaagcuuuugcuugucaugcagguacucucagagcgcucauagccauuauaagac
cuaaaaaggcagugcucugcggggaucccaaacagugcgguuuuuuuaacaugaugugcc
ugaaagugcauuuuaaccacgagauuugcacacaagucuuccacaaaagcaucucucgcc
guugcacuaaaucugugacuucggucgucucaaccuuguuuuacgacaaaaaaaugagaa
cgacgaauccgaaagagacuaagauugugauugacacuaccggcaguaccaaaccuaagc
aggacgaucucauucucacuuguuucagagggugggugaagcaguugcaaauagauuaca
aaggcaacgaaauaaugacggcagcugccucucaagggcugacccguaaagguguguaug
ccguucgguacaaggugaaugaaaauccucuguacgcacccaccucagaacaugugaacg
uccuacugacccgcacggaggaccgcaucguguggaaaacacuagccggcgacccaugga
uaaaaacacugacugccaaguacccugggaauuucacugccacgauagaggaguggcaag
cagagcaugaugccaucaugaggcacaucuuggagagaccggacccuaccgacgucuucc
agaauaaggcaaacguguguugggccaaggcutuagugccggugcugaagaccgcuggca
uagacaugaccacugaacaauggaacacuguggautauuuugaaacggacaaagcucacu
cagcagagauaguauugaaccaacuaugcgugagguucuuuggacucgaucuggacuccg
gucuauuuucugcacccacuguuccguuauccauuaggaauaaucacugggauaacuccc
cgucgccuaacauguacgggcugaauaaagaagugguccgucagcucucucgcagguacc
cacaacugccucgggcaguugccacuggaagagucuaugacaugaacacugguacacugc
gcaauuaugauccgcgcauaaaccuaguaccuguaaacagaagacugccucaugcuuuag
uccuccaccauaaugaacacccacagagugacuuuucuucauucgucagcaaauugaagg
gcagaacuguccugguggucggggaaaaguuguccgucccaggcaaaaugguugacuggu
ugucagaccggccugaggcuaccuucagagcucggcuggauuuaggcaucccaggugaug
ugcccaaauaugacauaauauuuguuaaugugaggaccccauauaaauaccaucacuauc
agcagugugaagaccaugccauuaagcutagcauguugaccaagaaagcuugucugcauc
ugaaucccggcggaaccugugucagcauagguuaugguuacgcugacagggccagcgaaa
gcaucauuggugcuauagcgcggcaguucaaguuuucccggguaugcaaaccgaaauccu
cacuugaagagacggaaguucuguuuguauucauuggguacgaucgcaaggcccguacgc
acaauccuuacaagcuuucaucaaccuugaccaacauuuauacagguuccagacuccacg
aagccggaugugcacccucauaucauguggugcgaggggauauugccacggccaccgaag
gagugauuauaaaugcugcuaacagcaaaggacaaccuggcggaggggugugcggagcgc
uguauaagaaauucccggaaagcuucgauuuacagccgaucgaaguaggaaaagcgcgac
uggucaaaggugcagcuaaacauaucauucaugccguaggaccaaacuucaacaaaguuu
cggagguugaaggugacaaacaguuggcagaggcuuaugaguccaucgcuaagauuguca
acgauaacaauuacaagucaguagcgauuccacuguuguccaccggcaucuuuuccggga
acaaagaucgacuaacccaaucauugaaccauuugcugacagcuuuagacaccacugaug
cagauguagccauauacugcagggacaagaaaugggaaaugacucucaaggaagcagugg
cuaggagagaagcaguggaggagauaugcauauccgacgacucuucagugacagaaccug
augcagagcuggugagggugcauccgaagaguucuuuggcuggaaggaagggcuacagca
caagcgauggcaaaacuuucucauauuuggaagggaccaaguuucaccaggcggccaagg
auauagcagaaauuaaugccauguggcccguugcaacggaggccaaugagcagguaugca
uguauauccucggagaaagcaugagcaguauuaggucgaaaugccccgucgaagagucgg
aagccuccacaccaccuagcacgcugccuugcuugugcauccaugccaugacuccagaaa
gaguacagcgccuaaaagccucacguccagaacaaatuacugugugcucauccuuuccau
ugccgaaguauagaaucacuggugugcagaagauccaaugcucccagccuauauuguucu
caccgaaagugccugcguauauucauccaaggaaguaucucguggaaacaccaccgguag
acgagacuccggagccaucggcagagaaccaauccacagaggggacaccugaacaaccac
cacuuauaaccgaggaugagaccaggacuagaacgccugagccgaucaucaucgaagagg
aagaagaggauagcauaaguuugcugucagauggcccgacccaccaggugcugcaagucg
aggcagacauucacgggccgcccucuguaucuagcucauccugguccauuccucaugcau
ccgacuuugauguggacaguuuauccauacuugacacccuggagggagcuagcgugacca
gcggggcaacgucagccgagacuaacucutacuucgcaaagaguauggaguuucuggcgc
gaccggugccugcgccucgaacaguauucaggaacccuccacaucccgcuccgcgcacaa
gaacaccgucacuugcacccagcagggccugcucgagaaccagccuaguuuccaccccgc
caggcgugaauagggugaucacuagagaggagcucgaggcgcuuaccccgucacgcacuc
cuagcaggucggucucgagaaccagccuggucuccaacccgccaggcguaaauaggguga
uuacaagagaggaguuugaggcguucguagcacaacaacaaugacgguuugaugcgggug
cauacaucuuuuccuccgacaccggucaagggcauuuacaacaaaaaucaguaaggcaaa
cggugcuauccgaagugguguuggagaggaccgaauuggagauuucguaugccccgcgcc
ucgaccaagaaaaagaagaauuacuacgcaagaaauuacaguuaaaucccacaccugcua
acagaagcagauaccaguccaggaagguggagaacaugaaagccauaacagcuagacgua
uucugcaaggccuagggcauuauuugaaggcagaaggaaaaguggagugcuaccgaaccc
ugcauccuguuccuuuguauucaucuagugugaaccgugccuuuucaagccccaaggucg
caguggaagccuguaacgccauguugaaagagaacuuuccgacuguggcuucuuacugua
uuauuccagaguacgaugccuauuuggacaugguugacggagcuucaugcugcuuagaca
cugccaguuuuugcccugcaaagcugcgcagcuuuccaaagaaacacuccuauuuggaac
ccacaauacgaucggcagugccuucagcgauccagaacacgcuccagaacguccuggcag
cugccacaaaaagaaauugcaaugucacgcaaaugagagaauugcccguauuggauucgg
cggccuuuaauguggaaugcuucaagaaauaugcguguaauaaugaauauugggaaacgu
uuaaagaaaaccccaucaggcuuacugaagaaaacgugguaaauuacauuaccaaauuaa
aaggaccaaaagcugcugcucuuuuugcgaagacacataauuugaauauguugcaggaca
uaccaauggacagguuuguaauggacuuaaagagagacgugaaagugacuccaggaacaa
aacauacugaagaacggcccaagguacaggugauccaggcugccgauccgcuagcaacag
cguaucugugcggaauccaccgagagcugguuaggagauuaaaugcgguccugcuuccga
acauucauacacuguuugauaugucggcugaagacuuugacgcuauuauagccgagcacu
uccagccuggggauuguguucuggaaacugacaucgcgucguuugauaaaagugaggacg
acgccauggcucugaccgcguuaaugauucuggaagacuuagguguggacgcagagcugu
ugacgcugauugaggcggcuuucggcgaaauuucaucaauacauuugcccacuaaaacua
aauuuaaauucggagccaugaugaaaucuggaauguuccucacacuguuugugaacacag
ucauuaacauuguaaucgcaagcagaguguugagagaacggcuaaccggaucaccaugug
cagcauucauuggagaugacaauaucgugaaaggagucaaaucggacaaauuaauggcag
acaggugcgccaccugguugaauauggaagucaagauuauagaugcuguggugggcgaga
aagcgccuuauuucuguggaggguuuauuuugugugacuccgugaccggcacagcgugcc
guguggcagacccccuaaaaaggcuguuuaagcuuggcaaaccucuggcagcagacgaug
aacaugaugaugacaggagaagggcauugcaugaagagucaacacgcuggaaccgagugg
guauucuuucagagcugugcaaggcaguagaaucaagguaugaaaccguaggaacuucca
ucauaguuauggccaugacuacucuagcuagcaguguuaaaucauucagcuaccugagag
gggccccuauaacucucuacggcuaaccugaauggacuacgacauagucuaguccgccgc
caccXuaaccgcggugucaaaaaccgcguggacgugguuaacaucccugcugggaggauc
agccguaauuauuauaauuggcuuggugcuggcuacuauuguggccauguacgugcugac
caaccagaaacauaauugaauacagcagcaauuuggcaagcugcuuacauagaacucgcg
gcgauuggcaugccgccuuaaaauuuuuauuuuauuuuucuuuucuuuuccgaaucggau
uuuguuuuuaauauuucaaaaaaaaaaaaaaaaaaaaaaaaaaaaaaaaaaaaaaaaaaa
aa
SEQ ID NO: 41-IL-12 Fusion Protein Elastin Linker
VPGVGVPGVG
SEQ ID NO: 42-
RNA encoding a human IL-12 fusion protein-p40-elastin-p35
CUGAGAUCACCGGUCAACAUGUGUCACCAGCAGUUGGUCAUCUCUUGGUUUUCCCUGGUU
UUUCUGGCAUCUCCCCUCGUGGCCAUAUGGGAACUGAAGAAAGAUGUUUAUGUCGUAGAA
UUGGAUUGGUAUCCGGAUGCCCCUGGAGAAAUGGUGGUCCUCACCUGUGACACCCCUGAA
GAAGAUGGUAUCACCUGGACCUUGGACCAGAGCAGUGAGGUCUUAGGCUCUGGCAAAACC
CUGACCAUCCAAGUCAAAGAGUUUGGAGAUGCUGGCCAGUACACCUGUCACAAAGGAGGC
GAGGUUCUAAGCCAUUCGCUCCUGCUGCUUCACAAAAAGGAAGAUGGAAUUUGGUCCACU
GAUAUUUUAAAGGACCAGAAAGAACCCAAAAAUAAGACCUUUCUAAGAUGCGAGGCCAAG
AAUUAUUCUGGACGUUUCACCUGCUGGUGGCUGACGACAAUCAGUACUGAUUUGACAUUC
AGUGUCAAAAGCAGCAGAGGCUCUUCUGACCCCCAAGGGGUGACGUGCGGAGCUGCUACA
CUCUCUGCAGAGAGAGUCAGAGGGGACAACAAGGAGUAUGAGUACUCAGUGGAGUGCCAG
GAGGACAGUGCCUGCCCAGCUGCUGAGGAGAGUCUGCCCAUUGAGGUCAUGGUGGAUGCC
GUUCACAAGCUCAAGUAUGAAAACUACACCAGCAGCUUCUUCAUCAGGGACAUCAUCAAA
CCUGACCCACCCAAGAACUUGCAGCUGAAGCCAUUAAAGAAUUCUCGGCAGGUGGAGGUC
AGCUGGGAGUACCCUGACACCUGGAGUACUCCACAUUCCUACUUCUCCCUGACAUUCUGC
GUUCAGGUCCAGGGCAAGAGCAAGAGAGAAAAGAAAGAUAGAGUCUUCACGGACAAGACC
UCAGCCACGGUCAUCUGCCGCAAAAAUGCCAGCAUUAGCGUGCGGGCCCAGGACCGCUAC
UAUAGCUCAUCUUGGAGCGAAUGGGCAUCUGUGCCCUGCAGUGUUCCUGGAGUAGGGGUA
CCUGGGGUGGGCGCCAGAAACCUCCCCGUGGCCACUCCAGACCCAGGAAUGUUCCCAUGC
CUUCACCACUCCCAAAACCUGCUGAGGGCCGUCAGCAACAUGCUCCAGAAGGCCAGACAA
ACUCUAGAAUUUUACCCUUGCACUUCUGAAGAGAUUGAUCAUGAAGAUAUCACAAAAGAU
AAAACCAGCACAGUGGAGGCCUGUUUACCAUUGGAAUUAACCAAGAAUGAGAGUUGCCUA
AAUUCCAGAGAGACCUCUUUCAUAACUAAUGGGAGUUGCCUGGCCUCCAGAAAGACCUCU
UUUAUGAUGGCCCUGUGCCUUAGUAGUAUUUAUGAAGACUUGAAGAUGUACCAGGUGGAG
UUCAAGACCAUGAAUGCAAAGCUGCUGAUGGAUCCUAAGAGGCAGAUCUUUCUAGAUCAA
AACAUGCUGGCAGUUAUUGAUGAGCUGAUGCAGGCCCUGAAUUUCAACAGUGAGACUGUG
CCACAAAAAUCCUCCCUUGAAGAACCGGAUUUUUAUAAAACUAAAAUCAAGCUCUGCAUA
CUUCUUCAUGCUUUCAGAAUUCGGGCAGUGACUAUUGAUAGAGUGAUGAGCUAUCUGAAU
GCUUCCUAAAAAGCGAGGUCCCUCCG

The following sequence (SEQ ID NO: 43) is formatted to signify vector backbone and cytokine open reading frames as follows: lower case letter signify the VEEV replicon backbone sequence; UPPER CASE letters signify the human IL-12 fusion open reading frame.

SEQ ID NO: 43:
VEEV + RNA human IL-12 fusion protein-p40-elastin-p35
auaggcggcgcaugagagaagcccagaccaauuaccuacccaaaauggagaaaguucacguugaca
ucgaggaagacagcccauuccucagagcuuugcagcggagcuucccgcaguuugagguagaagcc
aagcaggucacugauaaugaccaugcuaaugccagagcguuuucgcaucuggcuucaaaacugau
cgaaacggagguggacccauccgacacgauccuugacauuggaagugcgcccgcccgcagaaugu
auucuaagcacaaguaucauuguaucuguccgaugagaugugcggaagauccggacagauuguau
aaguaugcaacuaagcugaagaaaaacuguaaggaaauaacugauaaggaauuggacaagaaaau
gaaggagcuggccgccgucaugagcgacccugaccuggaaacugagacuaugugccuccacgacg
acgagucgugucgcuacgaagggcaagucgcuguuuaccaggauguauacgcgguugacggaccg
acaagucucuaucaccaagccaauaagggaguuagagucgccuacuggauaggcuuugacaccac
cccuuuuauguuuaagaacuuggcuggagcauauccaucauacucuaccaacugggccgacgaaa
ccguguuaacggcucguaacauaggccuaugcagcucugacguuauggagcggucacguagaggg
auguccauucuuagaaagaaguauuugaaaccauccaacaauguucuauucucuguuggcucgac
caucuaccacgagaagagggacuuacugaggagcuggcaccugccgucuguauuucacuuacgug
gcaagcaaaauuacacaugucggugugagacuauaguuaguugcgacggguacgucguuaaaaga
auagcuaucaguccaggccuguaugggaagccuucaggcuaugcugcuacgaugcaccgcgaggg
auucuugugcugcaaagugacagacacauugaacggggagagggucucuuuucccgugugcacgu
augugccagcuacauugugugaccaaaugacuggcauacuggcaacagaugucagugcggacgac
gcgcaaaaacugcugguugggcucaaccagcguauagucgucaacggucgcacccagagaaacac
caauaccaugaaaaauuaccuuuugcccquaguggcccaggcauuugcuaggugggcaaaggaau
auaaggaagaucaagaagaugaaaggccacuaggacuacgagauagacaguuagucauggggugu
uguugggcuuuuagaaggcacaagauaacaucuauuuauaagcgcccggauacccaaaccaucau
caaagugaacagcgauuuccacucauucgugcugcccaggauaggcaguaacacauuggagaucg
ggcugagaacaagaaucaggaaaauguuagaggagcacaaggagccgucaccucucauuaccgcc
gaggacguacaagaagcuaagugcgcagccgaugaggcuaaggaggugcgugaagccgaggaguu
gcgcgcagcucuaccaccuuuggcagcugauguugaggagcccacucuggaggcagacgucgacu
ugauguuacaagaggcuggggccggcucaguggagacaccucguggcuugauaaagguuaccagc
uacgauggcgaggacaagaucggcucuuacgcugugcuuucuccgcaggcuguacucaagaguga
aaaauuaucuugcauccacccucucgcugaacaagucauagugauaacacacucuggccgaaaag
ggcguuaugccguggaaccauaccaugguaaaguaguggugccagagggacaugcaauacccguc
caggacuuucaagcucugagugaaagugccaccauuguguacaacgaacgugaguucguaaacag
guaccugcaccauauugccacacauggaggagcgcugaacacugaugaagaauauuacaaaacug
ucaagcccagcgagcacgacggcgaauaccuguacgacaucgacaggaaacagugcgucaagaaa
gaacuagucacugggcuagggcucacaggcgagcugguggauccucccuuccaugaauucgccua
cgagagucugagaacacgaccagccgcuccuuaccaaguaccaaccauagggguguauggcgugc
caggaucaggcaagucuggcaucauuaaaagcgcagucaccaaaaaagaucuaguggugagcgcc
aagaaagaaaacugugcagaaauuauaagggacgucaagaaaaugaaagggcuggacgucaaugc
cagaacuguggacucagugcucuugaauggaugcaaacaccccguagagacccuguauauugacg
aagcuuuugcuugucaugcagguacucucagagcgcucauagccauuauaagaccuaaaaaggca
gugcucugcggggaucccaaacagugcgguuuuuuuaacaugaugugccugaaagugcauuuuaa
ccacgagauuugcacacaagucuuccacaaaagcaucucucgccguugcacuaaaucugugacuu
cggucgucucaaccuuguuuuacgacaaaaaaaugagaacgacgaauccgaaagagacuaagauu
gugauugacacuaccggcaguaccaaaccuaagcaggacgaucucauucucacuuguuucagagg
gugggugaagcaguugcaaauagauuacaaaggcaacgaaauaaugacggcagcugccucucaag
ggcugacccguaaagguguguaugccguucgguacaaggugaaugaaaauccucuguacgcaccc
accucagaacaugugaacguccuacugacccgcacggaggaccgcaucguguggaaaacacuagc
cggcgacccauggauaaaaacacugacugccaaguacccugggaauuucacugccacgauagagg
aguggcaagcagagcaugaugccaucaugaggcacaucuuggagagaccggacccuaccgacguc
uuccagaauaaggcaaacguguguugggccaaggcuuuagugccggugcugaagaccgcuggcau
agacaugaccacugaacaauggaacacuguggauuauuuugaaacggacaaagcucacucagcag
agauaguauugaaccaacuaugcgugagguucuuuggacucgaucuggacuccggucuauuuucu
gcacccacuguuccguuauccauuaggaauaaucacugggauaacuccccgucgccuaacaugua
cgggcugaauaaagaagugguccgucagcucucucgcagguacccacaacugccucgggcaguug
ccacuggaagagucuaugacaugaacacugguacacugcgcaauuaugauccgcgcauaaaccua
guaccuguaaacagaagacugccucaugcuuuaguccuccaccauaaugaacacccacagaguga
cuuuucuucauucgucagcaaauugaagggcagaacuguccugguggucggggaaaaguuguccg
ucccaggcaaaaugguugacugguugucagaccggccugaggcuaccuucagagcucggcuggau
uuaggcaucccaggugaugugcccaaauaugacauaauauuuguuaaugugaggaccccauauaa
auaccaucacuaucagcagugugaagaccaugccauuaagcuuagcauguugaccaagaaagcuu
gucugcaucugaaucccggcggaaccugugucagcauagguuaugguuacgcugacagggccagc
gaaagcaucauuggugcuauagcgcggcaguucaaguuuucccggguaugcaaaccgaaauccuc
acuugaagagacggaaguucuguuuguauucauuggguacgaucgcaaggcccguacgcacaauc
cuuacaagcuuucaucaaccuugaccaacauuuauacagguuccagacuccacgaagccggaugu
gcacccucauaucauguggugcgaggggauauugccacggccaccgaaggagugauuauaaaugc
ugcuaacagcaaaggacaaccuggcggaggggugugcggagcgcuguauaagaaauucccggaaa
gcuucgauuuacagccgaucgaaguaggaaaagcgcgacuggucaaaggugcagcuaaacauauc
auucaugccguaggaccaaacuucaacaaaguuucggagguugaaggugacaaacaguuggcaga
ggcuuaugaguccaucgcuaagauugucaacgauaacaauuacaagucaguagcgauuccacugu
uguccaccggcaucuuuuccgggaacaaagaucgacuaacccaaucauugaaccauuugcugaca
gcuuuagacaccacugaugcagauguagccauauacugcagggacaagaaaugggaaaugacucu
caaggaagcaguggcuaggagagaagcaguggaggagauaugcauauccgacgacucuucaguga
cagaaccugaugcagagcuggugagggugcauccgaagaguucuuuggcuggaaggaagggcuac
agcacaagcgauggcaaaacuuucucauauuuggaagggaccaaguuucaccaggcggccaagga
uauagcagaaauuaaugccauguggcccguugcaacggaggccaaugagcagguaugcauguaua
uccucggagaaagcaugagcaguauuaggucgaaaugccccgucgaagagucggaagccuccaca
ccaccuagcacgcugccuugcuugugcauccaugccaugacuccagaaagaguacagcgccuaaa
agccucacguccagaacaaauuacugugugcucauccuuuccauugccgaaguauagaaucacug
gugugcagaagauccaaugcucccagccuauauuguucucaccgaaagugccugcguauauucau
ccaaggaaguaucucguggaaacaccaccgguagacgagacuccggagccaucggcagagaacca
auccacagaggggacaccugaacaaccaccacuuauaaccgaggaugagaccaggacuagaacgc
cugagccgaucaucaucgaagaggaagaagaggauagcauaaguuugcugucagauggcccgacc
caccaggugcugcaagucgaggcagacauucacgggccgcccucuguaucuagcucauccugguc
cauuccucaugcauccgacuuugauguggacaguuuauccauacuugacacccuggagggagcua
gcgugaccagcggggcaacgucagccgagacuaacucuuacuucgcaaagaguauggaguuucug
gcgcgaccggugccugcgccucgaacaguauucaggaacccuccacaucccgcuccgcgcacaag
aacaccgucacuugcacccagcagggccugcucgagaaccagccuaguuuccaccccgccaggcg
ugaauagggugaucacuagagaggagcucgaggcgcuuaccccgucacgcacuccuagcaggucg
gucucgagaaccagccuggucuccaacccgccaggcguaaauagggugauuacaagagaggaguu
ugaggcguucguagcacaacaacaaugacgguuugaugcgggugcauacaucuuuuccuccgaca
ccggucaagggcauuuacaacaaaaaucaguaaggcaaacggugcuauccgaagugguguuggag
aggaccgaauuggagauuucguaugccccgcgccucgaccaagaaaaagaagaauuacuacgcaa
gaaauuacaguuaaaucccacaccugcuaacagaagcagauaccaguccaggaagguggagaaca
ugaaagccauaacagcuagacguauucugcaaggccuagggcauuauuugaaggcagaaggaaaa
guggagugcuaccgaacccugcauccuguuccuuuguauucaucuagugugaaccgugccuuuuc
aagccccaaggucgcaguggaagccuguaacgccauguugaaagagaacuuuccgacuguggcuu
cuuacuguauuauuccagaguacgaugccuauuuggacaugguugacggagcuucaugcugcuua
gacacugccaguuuuugcccugcaaagcugcgcagcuuuccaaagaaacacuccuauuuggaacc
cacaauacgaucggcagugccuucagcgauccagaacacgcuccagaacguccuggcagcugcca
caaaaagaaauugcaaugucacgcaaaugagagaauugcccguauuggauucggcggccuuuaau
guggaaugcuucaagaaauaugcguguaauaaugaauauugggaaacguuuaaagaaaaccccau
caggcuuacugaagaaaacgugguaaauuacauuaccaaauuaaaaggaccaaaagcugcugcuc
uuuuugcgaagacacauaauuugaauauguugcaggacauaccaauggacagguuuguaauggac
uuaaagagagacgugaaagugacuccaggaacaaaacauacugaagaacggcccaagguacaggu
gauccaggcugccgauccgcuagcaacagcguaucugugcggaauccaccgagagcugguuagga
gauuaaaugcgguccugcuuccgaacauucauacacuguuugauaugucggcugaagacuuugac
gcuauuauagccgagcacuuccagccuggggauuguguucuggaaacugacaucgcgucguuuga
uaaaagugaggacgacgccauggcucugaccgcguuaaugauucuggaagacuuagguguggacg
cagagcuguugacgcugauugaggcggcuuucggcgaaauuucaucaauacauuugcccacuaaa
acuaaauuuaaauucggagccaugaugaaaucuggaauguuccucacacuguuugugaacacagu
cauuaacauuguaaucgcaagcagaguguugagagaacggcuaaccggaucaccaugugcagcau
ucauuggagaugacaauaucgugaaaggagucaaaucggacaaauuaauggcagacaggugcgcc
accugguugaauauggaagucaagauuauagaugcugugguguggcgagaaagcgccuuauuucu
guggaggguuuauuuuugugacuccgugaccggcacagcgugccguguggcagacccccuaaaaa
ggcuguuuaagcuuggcaaaccucuggcagcagacgaugaacaugaugaugacaggagaagggca
uugcaugaagagucaacacgcuggaaccgaguggguauucuuucagagcugugcaaggcaguaga
aucaagguaugaaaccguaggaacuuccaucauaguuauggccaugacuacucuagcuagcagug
uuaaaucauucagcuaccugagaggggccccuauaacucucuacggcuaaccugaauggacuacg
acauagucuaguccgccaagCUGAGAUCACCGGUCAACAUGUGUCACCAGCAGUUGGUCAUCUCU
UGGUUUUCCCUGGUUUUUCUGGCAUCUCCCCUCGUGGCCAUAUGGGAACUGAAGAAAGAUGUUUA
UGUCGUAGAAUUGGAUUGGUAUCCGGAUGCCCCUGGAGAAAUGGUGGUCCUCACCUGUGACACCC
CUGAAGAAGAUGGUAUCACCUGGACCUUGGACCAGAGCAGUGAGGUCUUAGGCUCUGGCAAAACC
CUGACCAUCCAAGUCAAAGAGUUUGGAGAUGCUGGCCAGUACACCUGUCACAAAGGAGGCGAGGU
UCUAAGCCAUUCGCUCCUGCUGCUUCACAAAAAGGAAGAUGGAAUUUGGUCCACUGAUAUUUUAA
AGGACCAGAAAGAACCCAAAAAUAAGACCUUUCUAAGAUGCGAGGCCAAGAAUUAUUCUGGACGU
UUCACCUGCUGGUGGCUGACGACAAUCAGUACUGAUUUGACAUUCAGUGUCAAAAGCAGCAGAGG
CUCUUCUGACCCCCAAGGGGUGACGUGCGGAGCUGCUACACUCUCUGCAGAGAGAGUCAGAGGGG
ACAACAAGGAGUAUGAGUACUCAGUGGAGUGCCAGGAGGACAGUGCCUGCCCAGCUGCUGAGGAG
AGUCUGCCCAUUGAGGUCAUGGUGGAUGCCGUUCACAAGCUCAAGUAUGAAAACUACACCAGCAG
CUUCUUCAUCAGGGACAUCAUCAAACCUGACCCACCCAAGAACUUGCAGCUGAAGCCAUUAAAGA
AUUCUCGGCAGGUGGAGGUCAGCUGGGAGUACCCUGACACCUGGAGUACUCCACAUUCCUACUUC
UCCCUGACAUUCUGCGUUCAGGUCCAGGGCAAGAGCAAGAGAGAAAAGAAAGAUAGAGUCUUCAC
GGACAAGACCUCAGCCACGGUCAUCUGCCGCAAAAAUGCCAGCAUUAGCGUGCGGGCCCAGGACC
GCUACUAUAGCUCAUCUUGGAGCGAAUGGGCAUCUGUGCCCUGCAGUGUUCCUGGAGUAGGGGUA
CCUGGGGUGGGCGCCAGAAACCUCCCCGUGGCCACUCCAGACCCAGGAAUGUUCCCAUGCCUUCA
CCACUCCCAAAACCUGCUGAGGGCCGUCAGCAACAUGCUCCAGAAGGCCAGACAAACUCUAGAAU
UUUACCCUUGCACUUCUGAAGAGAUUGAUCAUGAAGAUAUCACAAAAGAUAAAACCAGCACAGUG
GAGGCCUGUUUACCAUUGGAAUUAACCAAGAAUGAGAGUUGCCUAAAUUCCAGAGAGACCUCUUU
CAUAACUAAUGGGAGUUGCCUGGCCUCCAGAAAGACCUCUUUUAUGAUGGCCCUGUGCCUUAGUA
GUAUUUAUGAAGACUUGAAGAUGUACCAGGUGGAGUUCAAGACCAUGAAUGCAAAGCUGCUGAUG
GAUCCUAAGAGGCAGAUCUUUCUAGAUCAAAACAUGCUGGCAGUUAUUGAUGAGCUGAUGCAGGC
CCUGAAUUUCAACAGUGAGACUGUGCCACAAAAAUCCUCCCUUGAAGAACCGGAUUUUUAUAAAA
CUAAAAUCAAGCUCUGCAUACUUCUUCAUGCUUUCAGAAUUCGGGCAGUGACUAUUGAUAGAGUG
AUGAGCUAUCUGAAUGCUUCCUAAAAAGCGAGGUCCCUCCGugauaaccgcggugucaaaaaccg
cguggacgugguuaacaucccugcugggaggaucagccagcaauuggcaagcugcuuacauagaa
cucgcggcgauuggcaugccgccuuaaaauuuuuauuuuauuuuuucuuuucuuuuccgaaucgg
auuuuguuuuuaauauuucaaaaaaaaaaaaaaaaaaaaaaaaaaaaaaaaaaaaaaaaaaaaa

The following sequence (SEQ ID NO: 44) is formatted to signify the sequence encoding the IL-12p40 subunit, a linker, and the IL-12 p35 subunit. The linker is shown in uppercase, bolded text. The sequence encoding IL-12p40 is shown in lower case letters. The sequence encoding IL-12p35 is shown in upper case letters

SEQ ID NO: 44: DNA construct encoding
mouse IL-12-p40-p35 fusion protein
atgtgccctcaaaaactcaccatcagttggtttgctattgtgctc
ttggtcagccctctcatggcaatgtgggaactcgaaaaagacgtg
tacgtcgtggaggttgactggacacccgacgcccctggagaaaca
gtaaaccttacatgtgatacccctgaggaggacgatataacctgg
acatctgatcaacgacatggcgtcattggatctgggaaaaccctg
accattacagtgaaagaatttctcgatgctggtcagtacacttgc
cacaagggaggtgaaactctcagtcattcacatcttctcctgcac
aaaaaagagaacggtatttggtctacagaaattctgaagaacttc
aagaacaaaactttcctgaagtgtgaggccccaaattatagcggc
cgattcacctgcagttggctcgtgcaacgaaacatggacctgaag
tttaatattaaaagtagttcttctagcccagatagccgggcagtg
acatgcggaatggcaagcctgtcagccgagaaagtgacactggat
cagagagattacgagaaatactccgtctcttgtcaggaggatgtg
acctgtcctactgccgaagagactctcccaatagaactcgctctt
gaagcaaggcagcagaataagtacgagaactactcaactagtttc
ttcataagggacatcatcaaaccagatcctcctaagaacctccaa
atgaaaccattgaaaaatagtcaagtggaagtttcttgggaatat
ccagactcctggtcaactccccactcttattttagcctcaagttc
ttcgtccgaatacagcggaagaaggaaaaaatgaaagagacagaa
gaaggatgtaaccagaaaggcgccttcctggtcgaaaagacttcc
acagaggtacaatgcaaggggggtaatgtctgtgtgcaagctcaa
gatcggtattataactccagctgctcaaaatgggcctgcgttcca
tgtcgagtccggagcGTACCTGGAGTCGGAGTACCCGGAGTTGGG
AGGGTTATTCCAGTGAGTGGCCCCGCTCGATGCCTGTCACAAAGC
AGAAACCTGCTTAAAACTACTGACGACATGGTTAAGACCGCTCGG
GAAAAATTGAAGCACTATAGTTGTACAGCAGAAGACATAGACCAT
GAAGACATTACCAGGGATCAGACAAGCACTTTGAAGACCTGCTTG
CCCCTTGAGCTGCACAAAAATGAATCTTGCCTCGCTACACGAGAA
ACAAGTAGTACTACTCGGGGGAGCTGCCTTCCCCCTCAGAAAACT
TCCTTGATGATGACACTTTGCCTTGGCAGCATCTATGAAGATCTT
AAAATGTATCAGACAGAATTTCAGGCCATCAATGCAGCACTGCAA
AACCACAATCATCAGCAAATCATTCTCGACAAAGGTATGCTTGTT
GCAATAGACGAGCTGATGCAATCCCTGAATCATAACGGCGAAACA
CTGCGGCAAAAACCACCTGTAGGCGAAGCCGATCCTTACCGAGTC
AAAATGAAGTTGTGCATCCTCTTGCACGCCTTTAGTACACGAGTT
GTTACTATAAACAGAGTCATGGGTTACTTGAGCAGTGCA

The following sequence (SEQ ID NO: 45) is formatted to signify the sequence encoding the IL-12p40 subunit, a linker, and the IL-12 p35 subunit. The linker is shown in uppercase, bolded text. The sequence encoding IL-12p40 is shown in lower case letters. The sequence encoding IL-12p35 is shown in upper case letters.

SEQ ID NO: 45: RNA encoding mouse
IL-12 - p40-p35 fusion protein
augugcccucaaaaacucaccaucaguugguuugcuauugugcuc
uuggucagcccucucauggcaaugugggaacucgaaaaagacgug
uacgucguggagguugacuggacacccgacgccccuggagaaaca
guaaaccuuacaugugauaccccugaggaggacgauauaaccugg
acaucugaucaacgacauggcgucauuggaucugggaaaacccug
accauuacagugaaagaauuucucgaugcuggucaguacacuugc
cacaagggaggugaaacucucagucauucacaucuucuccugcac
aaaaaagagaacgguauuuggucuacagaaauucugaagaacuuc
aagaacaaaacuuuccugaagugugaggccccaaauuauagcggc
cgauucaccugcaguuggcucgugcaacgaaacauggaccugaag
uuuaauauuaaaaguaguucuucuagcccagauagccgggcagug
acaugcggaauggcaagccugucagccgagaaagugacacuggau
cagagagauuacgagaaauacuccgucucuugucaggaggaugug
accuguccuacugccgaagagacucucccaauagaacucgcucuu
gaagcaaggcagcagaauaaguacgagaacuacucaacuaguuuc
uucauaagggacaucaucaaaccagauccuccuaagaaccuccaa
augaaaccauugaaaaauagucaaguggaaguuucuugggaauau
ccagacuccuggucaacuccccacucuuauuuuagccucaaguuc
uucguccgaauacagcggaagaaggaaaaaaugaaagagacagaa
gaaggauguaaccagaaaggcgccuuccuggucgaaaagacuucc
acagagguacaaugcaagggggguaaugucugugugcaagcucaa
gaucgguauuauaacuccagcugcucaaaaugggccugcguucca
ugucgaguccggagcGUACCUGGAGUCGGAGUACCCGGAGUUGGG
AGGGUUAUUCCAGUGAGUGGCCCCGCUCGAUGCCUGUCACAAAGC
AGAAACCUGCUUAAAACUACUGACGACAUGGUUAAGACCGCUCGG
GAAAAAUUGAAGCACUAUAGUUGUACAGCAGAAGACAUAGACCAU
GAAGACAUUACCAGGGAUCAGACAAGCACUUUGAAGACCUGCUUG
CCCCUUGAGCUGCACAAAAAUGAAUCUUGCCUCGCUACACGAGAA
ACAAGUAGUACUACUCGGGGGAGCUGCCUUCCCCCUCAGAAAACU
UCCUUGAUGAUGACACUUUGCCUUGGCAGCAUCUAUGAAGAUCUU
AAAAUGUAUCAGACAGAAUUUCAGGCCAUCAAUGCAGCACUGCAA
AACCACAAUCAUCAGCAAAUCAUUCUCGACAAAGGUAUGCUUGUU
GCAAUAGACGAGCUGAUGCAAUCCCUGAAUCAUAACGGCGAAACA
CUGCGGCAAAAACCACCUGUAGGCGAAGCCGAUCCUUACCGAGUC
AAAAUGAAGUUGUGCAUCCUCUUGCACGCCUUUAGUACACGAGUU
GUUACUAUAAACAGAGUCAUGGGUUACUUGAGCAGUGCA
SEQ ID NO: 46
MAGE-A1 Amino Acid Sequence
MPLEQRSQHCKPEEGLEARGEALGLVGAQAPATEEQEAASSSSTL
VEVTLGEVPAAESPDPPQSPQGASSLPTTMNYPLWSQSYEDSSNQ
EEEGPSTFPDLESEFQAALSRKVAELVQFLLLKYRAREPVTKAEM
LGSVVGNWQYFFPVIFSKASSSLQLVFGIELMEVDPTGHLYIFAT
CLGLSYDGLLGDNQIMPKAGLLIIVLAIIAREGDCAPEEKIWEEL
SVLEVFEGREDSILGDPKKLLTQHFVQENYLEYRQVPGSDPACYE
FLWGPRALVETSYVKVLHHMVKISGGPHISYPPLHEWVLREGEE
SEQ ID NO: 47
melanoma-associated antigen 3 (MAGE-A3)
[Homo sapiens]
NCBI Reference Sequence: NP_005353.1
MPLEQRSQHCKPEEGLEARGEALGLVGAQAPATEEQEAASSSSTL
VEVTLGEVPAAESPDPPQSPQGASSLPTTMNYPLWSQSYEDSSNQ
EEEGPSTFPDLESEFQAALSRKVAELVHFLLLKYRAREPVTKAEM
LGSVVGNWQYFFPVIFSKASSSLQLVFGIELMEVDPIGHLYIFAT
CLGLSYDGLLGDNQIMPKAGLLIIVLAIIAREGDCAPEEKIWEEL
SVLEVFEGREDSILGDPKKLLTQHFVQENYLEYRQVPGSDPACYE
FLWGPRALVETSYVKVLHHMVKISGGPHISYPPLHEWVLREGEE
SEQ ID NO: 48
TRP-1 Amino Acid Sequence
MKSYNVLPLAYISLFLMLFYQVWAQFPRECANIEALRRGVCCPDL
LPSSGPGTDPCGSSSGRGRCVAVIADSRPHSRHYPHDGKDDREAW
PLRFFNRTCQCNDNFSGHNCGTCRPGWRGAACNQKILTVRRNLLD
LSPEEKSHFVRALDMAKRTTHPQFVIATRRLEDILGPDGNTPQFE
NISVYNYFVWTHYYSVKKTFLGTGQESFGDVDFSHEGPAFLTWHR
YHLLQLERDMQEMLQEPSFSLPYWNFATGKNVCDVCTDDLMGSRS
NFDSTLISPNSVFSQWRVVCESLEEYDTLGTLCNSTEGGPIRRNP
AGNVGRPAVQRLPEPQDVTQCLEVRVFDTPPFYSNSTDSFRNTVE
GYSAPTGKYDPAVRSLHNLAHLFLNGTGGQTHLSPNDPIFVLLHT
FTDAVFDEWLRRYNADISTFPLENAPIGHNRQYNMVPFWPPVTNT
EMFVTAPDNLGYMYEVQWPGQEFTVSEIITIAVVAALLLVAAIFG
VASCLIRSRSTKNEANQPLLTDHYQRYAEDYEELPNPNHSMV
SEQ ID NOS: 49
Prostein AAK54386.1 prostein [Homo sapiens]
MVQRLWVSRLLRHRKAQLLLVNLLTFGLEVCLAAGITYVPPLLLE
VGVEEKFMTMVLGIGPVLGLVCVPLLGSASDHWRGRYGRRRPFIW
ALSLGILLSLFLIPRAGWLAGLLCPDPRPLELALLILGVGLLDFC
GQVCFTPLEALLSDLFRDPDHCRQAYSVYAFMISLGGCLGYLLPA
IDWDTSALAPYLGTQEECLFGLLTLIFLTCVAATLLVAEEAALGP
TEPAEGLSAPSLSPHCCPCRARLAFRNLGALLPRLHQLCCRMPRT
LRRLFVAELCSWMALMTFTLFYTDFVGEGLYQGVPRAEPGTEARR
HYDEGVRMGSLGLFLQCAISLVFSLVMDRLVQRFGTRAVYLASVA
AFPVAAGATCLSHSVAVVTASAALTGFTFSALQILPYTLASLYHR
EKQVFLPKYRGDTGGASSEDSLMTSFLPGPKPGAPFPNGHVGAGG
SGLLPPPPALCGASACDVSVRVVVGEPTEARVVPGRGICLDLAIL
DSAFLLSQVAPSLFMGSIVQLSQSVTAYMVSAAGLGLVAIYFATQ
VVFDKSDLAKYSA
SEQ ID NOS: 50-94:
See Table 3. Cancer-Associated Proteins.

What is claimed is:

1. A composition, wherein the composition comprises:
a lipid carrier, wherein a surface of the lipid carrier comprises cationic lipids; and
a nucleic acid encoding for: (i) an RNA-dependent RNA polymerase; and (ii) an IL-12,
wherein the surface of the lipid carrier and the nucleic acid form a lipid carrier-nucleic acid complex.

2. The composition of claim 1, wherein the IL-12 further comprises a linker between a p35 subunit and a p40 subunit.

3. The composition of claim 1, wherein the composition further comprises an innate immune response modulator. innate immune response modulator comprises a pattern recognition receptor (PRR) agonist or a nucleic acid encoding for a pattern recognition receptor (PRR) agonist.

4. The composition of claim 3, wherein the innate immune response modulator comprises a pattern recognition receptor (PRR) agonist or a nucleic acid encoding for a pattern recognition receptor (PRR) agonist.

5. The composition of claim 4, wherein the PRR agonist is RIG-I.

6. The composition of claim 4, wherein the PRR agonist is a TLR3 agonist or a TLR4 agonist.

7. The composition of claim 6, wherein the TLR4 agonist is glucopyranosyl lipid A (GLA), monophosphoryl lipid A (MPLA), lipopolysaccharide (LPS), or a derivative thereof.

8. The composition of claim 2, wherein the linker comprises 14 to 18 amino acids.

9. The composition of claim 1, wherein the RNA-dependent RNA polymerase includes a sub-genome of an alphavirus.

10. The composition of claim 1, wherein the nucleic acid comprises an RNA sequence of SEQ ID NO: 28.

11. The composition of claim 1, wherein the lipid carrier comprises a cationic lipid and a hydrophobic core.

12. The composition of claim 11, wherein the cationic lipid is 1,2-dioleoyloxy-3 (trimethylammonium)propane, 3β-[N—(N′,N′-dimethylaminoethane) carbamoyl]cholesterol, dimethyldioctadecylammonium, 1,2-dimyristoyl 3-trimethylammoniumpropane, dipalmitoyl(C16:0)trimethyl ammonium propane, distearoyltrimethylammonium propane, N-[1-(2,3-dioleyloxy)propyl]N,N,Ntrimethylammonium, chloride, N,N-dioleoyl-N,N-dimethylammonium chloride (DODAC), 1,2-dioleoyl-sn-glycero-3-ethylphosphocholine, 1,2-dioleoyl-3-dimethylammonium-propane, 1,2-dilinoleyloxy-3-dimethylaminopropane, 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol), tetrakis(8-methylnonyl) 3,3′,3″,3′″-(((methylazanediyl) bis(propane-3,1 diyl))bis(azanetriyl))tetrapropionate, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate, ((4-hydroxybutyl)azanediyl)bis(hexane-6,1-diyl)bis(2-hexyldecanoate), 2-[(polyethylene glycol)-2000]-N,N-ditetradecylacetamide, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol, bis(2-(dodecyldisulfanyl)ethyl) 3,3′-((3-methyl-9-oxo-10-oxa-13,14-dithia-3,6-diazahexacosyl)azanediyl)dipropionate, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide, 3,6-bis(4-(bis(2-hydroxydodecyl)amino)butyl)piperazine-2,5-dione, 3β-[N—(N′,N′-dimethylaminoethane)-carbamoyl]cholesterol, (6Z,9Z,28Z,31Z)-heptatriaconta-6,9,28,31-tetraen-19-yl 4-(dimethylamino) butanoate, 1,2-dioleoyl-sn-glycero-3-phosphoethanolamine, 2,3-dioleyloxy-N-[2-(sperminecarboxamido)ethyl]-N,N-dimethyl-1-propanaminium trifluoroacetate, 1,2-distearoyl-sn-glycero-3-phosphocholine, ethylphosphatidylcholine, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoateheptadecan-9-yl 8-((2-hydroxyethyl)(6-oxo-6-(undecyloxy)hexyl)amino) octanoate, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate), or N1,N3,N5-tris(3-(didodecylamino)propyl)benzene-1,3,5-tricarboxamide.

13. The composition of claim 11, wherein the hydrophobic core comprises an oil.

14. The composition of claim 13, wherein the oil is in liquid phase at 25 degrees Celsius.

15. The composition of claim 13, wherein the oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E.

16. The composition of claim 15, wherein the triglyceride is capric triglyceride, caprylic triglyceride, a caprylic and capric triglyceride, a triglyceride ester, or myristic acid triglycerin.

17. The composition of claim 1, wherein the lipid carrier further comprises an inorganic particle.

18. The composition of claim 17, wherein the inorganic particle comprises a metal, a metal salt, a metal oxide, a metal hydroxide, or a metal phosphate.

19. The composition of claim 1, wherein lipid nanoparticle is characterized as having a z-average diameter particle size measurement of 20 nm to about 60 nm when measured using dynamic light scattering.

20. The composition of claim 1, wherein the composition is lyophilized.

21. The composition of claim 1, wherein the composition is in a liquid, semi-liquid, solution, propellant, or powder dosage form.

22. The composition of claim 1, wherein the composition is formulated as a suspension.

23. A method of modulating an immune response in a subject, the method comprising: administering to the subject the composition of claim 1, thereby modulating an immune response in the subject.


SEQUENCE LISTING (97 sequences)

SEQ ID NO: 1 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 236 aa, Mus musculus
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 4 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 5 - protein, 534 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLILLHAFRIRAVTIDRVMSYLNAS

SEQ ID NO: 6 - protein, 538 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 7 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 8 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 9 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 12 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 14 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 15 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 16 - RNA, 1643 nt, synthetic construct
  source 1..1643: mol_type = other RNA
ctgagatcaccgggacgcgtcaacatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaaag

SEQ ID NO: 17 - RNA, 103 nt, synthetic construct
  source 1..103: mol_type = other RNA
ccatcctgtttttttccctttttttttttcttttttttttttttttttttttttttttttttttctcctttttttttcctctttttttccttttctttccttt

SEQ ID NO: 18 - RNA, 109 nt, synthetic construct
  source 1..109: mol_type = other RNA
ggccatcctgtttttttccctttttttttttcttttttttttttttttttttttttttttttttttctcctttttttttcctctttttttccttttctttccttttcta

SEQ ID NO: 19 - RNA, 60 nt, synthetic construct
  source 1..60: mol_type = other RNA
cccccccccccccccccccccccccccccccccccccccccccccccccccccccccccc

SEQ ID NO: 20 - RNA, 100 nt, synthetic construct
  source 1..100: mol_type = other RNA
gggggggggggggggggggggggggggggggggggggggggggggggggggggggggggggggggggggggggggggggggggggggggggggggggggg

SEQ ID NO: 21 - RNA, 165 nt, synthetic construct
  source 1..165: mol_type = other RNA
  modified_base 1: mod_base = OTHER; note = Thymine
  modified_base 4: mod_base = OTHER; note = Thymine
  modified_base 7: mod_base = OTHER; note = Thymine
  modified_base 10: mod_base = OTHER; note = Thymine
  modified_base 11: mod_base = OTHER; note = Thymine
  modified_base 21: mod_base = OTHER; note = Thymine
  modified_base 22: mod_base = OTHER; note = Thymine
  modified_base 25: mod_base = OTHER; note = Thymine
tgctgctgcttgcaagcagcttgataccagacaaagctgggaatagaaacttcgtattttcaaagttttctttaatatattgcaaataatgcctaaccacctagggcaggattagggttccggagttcaaccaattagtccttaatcagggcactgtatccgact

SEQ ID NO: 22 - RNA, 140 nt, synthetic construct
  source 1..140: mol_type = other RNA
agtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 23 - RNA, 32 nt, synthetic construct
  source 1..32: mol_type = other RNA
  modified_base 1: mod_base = OTHER; note = 5' triphosphate modified nucleotide
ggatcgatcgatcgttcgcgatcgatcgatcc

SEQ ID NO: 24 - RNA, 99 nt, synthetic construct
  source 1..99: mol_type = other RNA
gacgaagaccacaaaaccagataaaaaaaaaaaaaaaaaaaaaaaaaataattttttttttttttttttttttttttatctggttttgtggtcttcgtc

SEQ ID NO: 25 - RNA, 24 nt, synthetic construct
  source 1..24: mol_type = other RNA
  modified_base 1: mod_base = OTHER; note = 5' triphosphate modified nucleotide
ggacgtacgtttcgacgtacgtcc

SEQ ID NO: 26 - RNA, 87 nt, synthetic construct
  source 1..87: mol_type = other RNA
  modified_base 1: mod_base = OTHER; note = 5' triphosphate modified nucleotide
agcaaaagcagggtgacaaagacataatggatccaaacactgtgtcaagctttcaggtagattgctttctttggcatgtccgcaaac

SEQ ID NO: 27 - DNA, 122 bp, synthetic construct
  source 1..122: mol_type = other DNA
  modified_base 1: mod_base = OTHER; note = 5' triphosphate modified nucleotide
taatacgactcactataggccatcctgtttttttcccttttttttttctttttttttttttttttttttttttttttttttttctcctttttttttcctctttttttccttttctttccttt

SEQ ID NO: 28 - RNA, 7561 nt, Venezuelan equine encephalitis virus
  source 1..7561: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 29 - protein, 51 aa, Venezuelan equine encephalitis virus
RELPVLDSAAFNVECFKKYACNNEYWETFKENPIRLTEENVVNYITKLKGP

SEQ ID NO: 30 - protein, 41 aa, Venezuelan equine encephalitis virus
TQMRELPVLDSAAFNVECFKKYACNNEYWETFKENPIRLTE

SEQ ID NO: 31 - protein, 536 aa, Venezuelan equine encephalitis virus
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 32 - RNA, 9504 nt, synthetic construct
  source 1..9504: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagctgagatcaccgggacgcgtcaacatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaaagtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 33 - DNA, 6569 bp, unidentified
  source 1..6569: mol_type = unassigned DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacaagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtcagatccagatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - RNA, 7862 nt, synthetic construct
  source 1..7862: mol_type = other RNA
  misc_difference 7562: note = a, g, u, or c
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 35 - RNA, 7862 nt, synthetic construct; identical to SEQ ID NO: 34
  source 1..7862: mol_type = other RNA
  misc_difference 7562: note = a, g, u, or c

SEQ ID NO: 36 - RNA, 7862 nt, synthetic construct
  source 1..7862: mol_type = other RNA
  misc_difference 7562: note = a, g, u, or c
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 37 - RNA, 7862 nt, synthetic construct; identical to SEQ ID NO: 34
  source 1..7862: mol_type = other RNA
  misc_difference 7562: note = a, g, u, or c

SEQ ID NO: 38 - RNA, 7862 nt, synthetic construct; identical to SEQ ID NO: 34
  source 1..7862: mol_type = other RNA
  misc_difference 7562: note = a, g, u, or c

SEQ ID NO: 39 - RNA, 11701 nt, synthetic construct
  source 1..11701: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 40 - RNA, 7862 nt, synthetic construct
  source 1..7862: mol_type = other RNA
  misc_difference 7565: note = a, g, u, or c
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccntaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 41 - protein, 10 aa, synthetic construct
VPGVGVPGVG

SEQ ID NO: 42 - RNA, 1646 nt, synthetic construct
  source 1..1646: mol_type = other RNA
ctgagatcaccggtcaacatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccg

SEQ ID NO: 43 - RNA, 9507 nt, synthetic construct
  source 1..9507: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagctgagatcaccggtcaacatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccgtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 44 - DNA, 1614 bp, synthetic construct
  source 1..1614: mol_type = other DNA
atgtgccctcaaaaactcaccatcagttggtttgctattgtgctcttggtcagccctctcatggcaatgtgggaactcgaaaaagacgtgtacgtcgtggaggttgactggacacccgacgcccctggagaaacagtaaaccttacatgtgatacccctgaggaggacgatataacctggacatctgatcaacgacatggcgtcattggatctgggaaaaccctgaccattacagtgaaagaatttctcgatgctggtcagtacacttgccacaagggaggtgaaactctcagtcattcacatcttctcctgcacaaaaaagagaacggtatttggtctacagaaattctgaagaacttcaagaacaaaactttcctgaagtgtgaggccccaaattatagcggccgattcacctgcagttggctcgtgcaacgaaacatggacctgaagtttaatattaaaagtagttcttctagcccagatagccgggcagtgacatgcggaatggcaagcctgtcagccgagaaagtgacactggatcagagagattacgagaaatactccgtctcttgtcaggaggatgtgacctgtcctactgccgaagagactctcccaatagaactcgctcttgaagcaaggcagcagaataagtacgagaactactcaactagtttcttcataagggacatcatcaaaccagatcctcctaagaacctccaaatgaaaccattgaaaaatagtcaagtggaagtttcttgggaatatccagactcctggtcaactccccactcttattttagcctcaagttcttcgtccgaatacagcggaagaaggaaaaaatgaaagagacagaagaaggatgtaaccagaaaggcgccttcctggtcgaaaagacttccacagaggtacaatgcaaggggggtaatgtctgtgtgcaagctcaagatcggtattataactccagctgctcaaaatgggcctgcgttccatgtcgagtccggagcgtacctggagtcggagtacccggagttgggagggttattccagtgagtggccccgctcgatgcctgtcacaaagcagaaacctgcttaaaactactgacgacatggttaagaccgctcgggaaaaattgaagcactatagttgtacagcagaagacatagaccatgaagacattaccagggatcagacaagcactttgaagacctgcttgccccttgagctgcacaaaaatgaatcttgcctcgctacacgagaaacaagtagtactactcgggggagctgccttccccctcagaaaacttccttgatgatgacactttgccttggcagcatctatgaagatcttaaaatgtatcagacagaatttcaggccatcaatgcagcactgcaaaaccacaatcatcagcaaatcattctcgacaaaggtatgcttgttgcaatagacgagctgatgcaatccctgaatcataacggcgaaacactgcggcaaaaaccacctgtaggcgaagccgatccttaccgagtcaaaatgaagttgtgcatcctcttgcacgcctttagtacacgagttgttactataaacagagtcatgggttacttgagcagtgca

SEQ ID NO: 45 - RNA, 1614 nt, synthetic construct
  source 1..1614: mol_type = other RNA
atgtgccctcaaaaactcaccatcagttggtttgctattgtgctcttggtcagccctctcatggcaatgtgggaactcgaaaaagacgtgtacgtcgtggaggttgactggacacccgacgcccctggagaaacagtaaaccttacatgtgatacccctgaggaggacgatataacctggacatctgatcaacgacatggcgtcattggatctgggaaaaccctgaccattacagtgaaagaatttctcgatgctggtcagtacacttgccacaagggaggtgaaactctcagtcattcacatcttctcctgcacaaaaaagagaacggtatttggtctacagaaattctgaagaacttcaagaacaaaactttcctgaagtgtgaggccccaaattatagcggccgattcacctgcagttggctcgtgcaacgaaacatggacctgaagtttaatattaaaagtagttcttctagcccagatagccgggcagtgacatgcggaatggcaagcctgtcagccgagaaagtgacactggatcagagagattacgagaaatactccgtctcttgtcaggaggatgtgacctgtcctactgccgaagagactctcccaatagaactcgctcttgaagcaaggcagcagaataagtacgagaactactcaactagtttcttcataagggacatcatcaaaccagatcctcctaagaacctccaaatgaaaccattgaaaaatagtcaagtggaagtttcttgggaatatccagactcctggtcaactccccactcttattttagcctcaagttcttcgtccgaatacagcggaagaaggaaaaaatgaaagagacagaagaaggatgtaaccagaaaggcgccttcctggtcgaaaagacttccacagaggtacaatgcaaggggggtaatgtctgtgtgcaagctcaagatcggtattataactccagctgctcaaaatgggcctgcgttccatgtcgagtccggagcgtacctggagtcggagtacccggagttgggagggttattccagtgagtggccccgctcgatgcctgtcacaaagcagaaacctgcttaaaactactgacgacatggttaagaccgctcgggaaaaattgaagcactatagttgtacagcagaagacatagaccatgaagacattaccagggatcagacaagcactttgaagacctgcttgccccttgagctgcacaaaaatgaatcttgcctcgctacacgagaaacaagtagtactactcgggggagctgccttccccctcagaaaacttccttgatgatgacactttgccttggcagcatctatgaagatcttaaaatgtatcagacagaatttcaggccatcaatgcagcactgcaaaaccacaatcatcagcaaatcattctcgacaaaggtatgcttgttgcaatagacgagctgatgcaatccctgaatcataacggcgaaacactgcggcaaaaaccacctgtaggcgaagccgatccttaccgagtcaaaatgaagttgtgcatcctcttgcacgcctttagtacacgagttgttactataaacagagtcatgggttacttgagcagtgca

SEQ ID NO: 46 - protein, 314 aa, unidentified
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVQFLLLKYRAREPVTKAEMLGSVVGNWQYFFPVIFSKASSSLQLVFGIELMEVDPTGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 47 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 48 - protein, 537 aa, unidentified
MKSYNVLPLAYISLFLMLFYQVWAQFPRECANIEALRRGVCCPDLLPSSGPGTDPCGSSSGRGRCVAVIADSRPHSRHYPHDGKDDREAWPLRFFNRTCQCNDNFSGHNCGTCRPGWRGAACNQKILTVRRNLLDLSPEEKSHFVRALDMAKRTTHPQFVIATRRLEDILGPDGNTPQFENISVYNYFVWTHYYSVKKTFLGTGQESFGDVDFSHEGPAFLTWHRYHLLQLERDMQEMLQEPSFSLPYWNFATGKNVCDVCTDDLMGSRSNFDSTLISPNSVFSQWRVVCESLEEYDTLGTLCNSTEGGPIRRNPAGNVGRPAVQRLPEPQDVTQCLEVRVFDTPPFYSNSTDSFRNTVEGYSAPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYMYEVQWPGQEFTVSEIITIAVVAALLLVAAIFGVASCLIRSRSTKNEANQPLLTDHYQRYAEDYEELPNPNHSMV

SEQ ID NO: 49 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 50 - protein, 9 aa, synthetic construct
KCDICTDEY

SEQ ID NO: 51 - protein, 9 aa, synthetic construct
YMDGTMSQV

SEQ ID NO: 52 - protein, 9 aa, synthetic construct
MLLAYLYQL

SEQ ID NO: 53 - protein, 9 aa, synthetic construct
AFLPWHRLF

SEQ ID NO: 54 - protein, 9 aa, synthetic construct
SEIWRDIDF

SEQ ID NO: 55 - protein, 9 aa, synthetic construct
YLEPGPVTA

SEQ ID NO: 56 - protein, 9 aa, synthetic construct
KTWGQUWQV

SEQ ID NO: 57 - protein, 9 aa, synthetic construct
ITDQVPFSV

SEQ ID NO: 58 - protein, 10 aa, synthetic construct
VLYRYGSFSV

SEQ ID NO: 59 - protein, 10 aa, synthetic construct
LLDGTATLRL

SEQ ID NO: 60 - protein, 9 aa, synthetic construct
ALLAVGATK

SEQ ID NO: 61 - protein, 9 aa, synthetic construct
MLGTHTMEV

SEQ ID NO: 62 - protein, 9 aa, synthetic construct
LIYRRRLMK

SEQ ID NO: 63 - protein, 9 aa, synthetic construct
ALNFPGSQK

SEQ ID NO: 64 - protein, 9 aa, synthetic construct
AAGIGILTV

SEQ ID NO: 65 - protein, 9 aa, synthetic construct
ILTVILGVL

SEQ ID NO: 66 - protein, 9 aa, synthetic construct
MSLQRQFLR

SEQ ID NO: 67 - protein, 9 aa, synthetic construct
SVYDFFVWL

SEQ ID NO: 68 - protein, 9 aa, synthetic construct
LLGPGRPYR

SEQ ID NO: 69 - protein, 9 aa, synthetic construct
YLSGANLNL

SEQ ID NO: 70 - protein, 9 aa, synthetic construct
KIFGSLAFL

SEQ ID NO: 71 - protein, 10 aa, synthetic construct
VMAGVGSPYV

SEQ ID NO: 72 - protein, 9 aa, synthetic construct
IISAVVGIL

SEQ ID NO: 73 - protein, 9 aa, synthetic construct
LLHETDSAV

SEQ ID NO: 74 - protein, 9 aa, synthetic construct
ALFDIESKV

SEQ ID NO: 75 - protein, 9 aa, synthetic construct
EADPTGHSY

SEQ ID NO: 76 - protein, 9 aa, synthetic construct
SLFRAVITK

SEQ ID NO: 77 - protein, 9 aa, synthetic construct
SAYGEPRKL

SEQ ID NO: 78 - protein, 9 aa, synthetic construct
KMVELVHFL

SEQ ID NO: 79 - protein, 10 aa, synthetic construct
YLOLVFGIEV

SEQ ID NO: 80 - protein, 9 aa, synthetic construct
EVDPIGHLY

SEQ ID NO: 81 - protein, 9 aa, synthetic construct
FLWGPRALV

SEQ ID NO: 82 - protein, 10 aa, synthetic construct
MEVDPIGHLY

SEQ ID NO: 83 - protein, 9 aa, synthetic construct
AARAVFLAL

SEQ ID NO: 84 - protein, 8 aa, synthetic construct
YRPRPRRY

SEQ ID NO: 85 - protein, 9 aa, synthetic construct
VLPDVFIRC

SEQ ID NO: 86 - protein, 9 aa, synthetic construct
QLSLLMWIT

SEQ ID NO: 87 - protein, 9 aa, synthetic construct
SLLMWITQC

SEQ ID NO: 88 - protein, 10 aa, synthetic construct
ASGPGGGAPR

SEQ ID NO: 89 - protein, 10 aa, synthetic construct
QDLTMKYQIF

SEQ ID NO: 90 - protein, 10 aa, synthetic construct
  VARIANT 1: note = May be absent
EAYGLDFYIL

SEQ ID NO: 91 - protein, 9 aa, synthetic construct
SYLDSGIHF

SEQ ID NO: 92 - protein, 9 aa, synthetic construct
ETVSEQSNV

SEQ ID NO: 93 - protein, 9 aa, synthetic construct
FPSDSWCYF

SEQ ID NO: 94 - protein, 9 aa, synthetic construct
EEKLIVVLF

SEQ ID NO: 95 - protein, 9 aa, unidentified
SYLDSGIHS

SEQ ID NO: 96 - protein, 9 aa, unidentified
ETVSEESNV

SEQ ID NO: 97 - protein, 9 aa, unidentified
EEKLSVVLF